(19) United States
(12) Patent Application Publication
(10) Pub. No.: US 2024/0207421 A1
(43) Pub. Date: Jun. 27, 2024

(54) NERVE TARGETING PEPTIDES AND METHODS OF USE

(71) Applicant: ALUME BIOSCIENCES, INC., San Diego, CA (US)

(72) Inventors: Michael WHITNEY, San Diego, CA (US); Quyen NGUYEN, San Diego, CA (US)

(21) Appl. No.: 18/254,565

(22) PCT Filed: Dec. 3, 2021
(86) PCT No.: PCT/US2021/061821

§ 371 Date: May 25, 2023

Related U.S. Application Data
(60) Provisional application No. 63/121,825, filed on Dec. 4, 2020.
(60) Provisional application No. 63/167,572, filed on Mar. 29, 2021.

(51) Int. Cl. A61K 47/64; A61K 41/00; A61K 49/00; C12N 15/10

(52) U.S. Cl. CPC A61K 47/64; A61K 41/0061; A61K 41/0071; A61K 41/0076; A61K 41/008; A61K 49/0056; C12N 15/1037

(57) ABSTRACT

The present disclosure provides nerve targeting peptides, including those comprising the amino acid sequence of any one of SEQ ID NOS: 1-710; conjugates comprising such nerve targeting peptides and a cargo molecule; compositions comprising said peptides and conjugates; and methods of said peptides and conjugates in surgical procedures and other applications.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 110267_404WO_Sequence_Listing.txt. The text file is 144 KB, was created on Nov. 18, 2021, and is being submitted electronically via EFS-Web.

### BACKGROUND

Preservation of human neurons and human nerves is one of the most important goals of any surgical procedure, because accidental transection of neuron or nerves leads to significant morbidity. Nerves are typically identified by their elongated whitish appearance and relationship to nearby structures or by electrophysiological studies. However, in instances such as trauma, tumor involvement, inflammation, or infection, nerve identification using these criteria can be difficult. Therefore, there is a need for methods of reliably and conclusively identifying neuron or nerves which overcome the deficiencies in the art.

One approach to neuron or nerve identification prior to direct exposure during surgery or confirmation of neuron or nerve identity in instances of uncertainty following direct exposure is electromyographic (EMG) monitoring. This technique, however, has the disadvantage of not providing visual feedback to the operating surgeon. Thus, even if a nerve has been identified in one location, either through accidental or purposeful stimulation, there is no visual guidance to the operating surgeon as to how far away from the stimulation site the nerve lies or the direction of travel the nerve takes away from the stimulation site. Furthermore, EMG only traces motor pathways, not sensory fibers. EMG fails if neuron or nerve conduction or neuromuscular transmission is temporarily blocked anywhere distal to the recording site. Such blockade easily occurs due to neuron or nerve compression, trauma, local anesthetics, or neuromuscular blockers.

Other approaches include neuron or nerve labeling, which primarily depend on retrograde or anterograde tracing of individually identified axonal tracts via the use of fluorescent dyes. However, methods of labeling neuron or nerves by locally applied fluorescent tracers have several disadvantages. First, this technique can label only one neuron or nerve fiber tract at a time, depending on where the dye has been injected. Second, this technique results in only limited labeling of fluorescent dyes along the axonal tracts, because retrograde axonal tracers typically accumulate in the neural cell body. Third, retrograde transport is relatively slow (on the order of millimeters per day) and therefore takes a long time to label human neuron or nerves, which are often longer than a meter, such as in the case of the sciatic neuron or nerve and its arborizations. Fourth, the application of fluorescent dyes to innervation targets such as direct intramuscular injections to label motor neuron or nerves is typically messy with a variable amount of the tracer dye remaining at the injection site. As dissection of neuron or nerves depends on accurate visualization of adjacent structures prior to encountering them, a surgical site that is contaminated with fluorescent dyes would not be desirable. Finally, the direct injection of the fluorescent dye itself may be damaging to the target organs or neuron or nerve of interest, either by mechanical damage or by the very high local concentration of dye and vehicle at the injection site.

There has been a need in the art to identify peptides capable of binding to human nerves and neurons, in order to facilitate surgical procedures and human nerve protection. The present disclosure provides peptide sequences that selectively bind to human nerves or neurons, as well as methods of using those peptides, for example to visualize nerves during surgical procedures, or to target drugs and other agents to nerves.

### BRIEF SUMMARY

In one aspect, the present disclosure provides a nerve targeting peptide conjugate comprising: a peptide comprising the amino acid sequence of any one of SEQ ID NOS: 1-710; and a cargo molecule. In embodiments, the present disclosure provides a nerve targeting peptide conjugate comprising: a peptide comprising the amino acid sequence of any one of SEQ ID NOS: 1-66; and a cargo molecule. In embodiments, the present disclosure provides a nerve targeting peptide conjugate comprising: a peptide comprising the amino acid sequence of any one of SEQ ID NOS: 1, 2, 4-17, 23, 24, 29, 31, 34, 35 37, 40, 41, 42, 44-47, 49, 50, 52-60, and 67-710 and a cargo molecule.

In some embodiments, the cargo molecule comprises a drug, fluorescent moiety, or photosensitizing agent.

In some embodiments, the cargo molecule is joined to the N-terminus or C-terminus of the peptide.

In some embodiments, the cargo molecule is joined to the peptide via a linker.

In some embodiments, the linker is a straight or branched-chain carbon linker, heterocyclic carbon linker, amino acid linker, lipophilic residue, peptide linker, peptide nucleic acid linker, hydrazone linker, SPDB disulfide, sulfo-SPDB, maleimidomethyl cyclohexane-1-carboxylate (MCC), aminohexanoic acid linker, polyether linker, or polyethylene glycol linker.

In some embodiments, the cargo molecule comprises a fluorescent moiety.

In some embodiments, the fluorescent moiety is a fluorescent protein, a fluorescent peptide, a fluorophore, or any combination thereof.

In some embodiments, the fluorescent moiety is selected from the group consisting of: a xanthene; a bimane; a coumarin; an aromatic amine; a benzofuran; a fluorescent cyanine; a carbazole; a dicyanomethylene pyrane; polymethine; oxabenzanthrane; pyrylium; carbostyl; perylene; acridone; quinacridone; rubrene; anthracene; coronene; phenanthrecene; pyrene; butadiene; stilbene; porphyrin; pthalocyanine; lanthanide metal chelate complexes; rare-earth metal chelate complexes; and derivatives thereof.

In some embodiments, the fluorescent moiety is selected from the group consisting of: 5-carboxyfluorescein; fluorescein-5-isothiocyanate; 6-carboxyfluorescein; tetramethylrhodamine-6-isothiocyanate; 5-carboxytetramethylrhodamine; 5-carboxy rhodol derivatives; tetramethyl and tetraethyl rhodamine; diphenyldimethyl and diphenyldiethyl rhodamine; dinaphthyl rhodamine; rhodamine 101 sulfonyl chloride; Cy3, Cy3B, Cy3.5, Cy5, Cy5.5, Cy 7, indocyanine green, IR800CW, cyan fluorescent protein (CFP), EGFP, 6-FAM, FAM, fluorescein, 5,6-dicarboxyfluorescein, 5-(and 6)-sulfofluorescein, sulfonefluorescein, succinyl fluorescein, 5-(and 6)-carboxy SNARF-1, carboxyfluorescein sulfonate, carboxyfluorescein zwitterion, carboxyfluorescein quaternary ammonium, carboxyfluorescein phosphonate, carboxyfluorescein GABA, carboxyfluorescein-cys-Cy5,5′(6′)-carboxyfluorescein, fluorescein glutathione, or any combination thereof.

In some embodiments, the cargo molecule comprises a photosensitizing agent.

In some embodiments, the photosensitizing agent is selected from the group consisting of: a porphyrin, chlorin, and dye.

In some embodiments, the photosensitizing agent is selected from the group consisting of: porphyrin, protoporfin IX, purlytin, verteporfin, HPPH, temoporfin, methylene blue, photofrin, protofrin, hematoporphyrin, Talaporfin, benzopophyrin derivative monoacid, 5-aminileuvolinic acid, Lutetium texaphyrin, metallophthalocyanine, metallo-naphthocyaninesulfobenzo-porphyrazine, metallo-naphthalocyanines, zinc tetrasulfophthalocyanine, bacteriochlorins, metallochlorins, chlorine derivative, Tetra(m-hydroxyphenyl)chlorin (mTHPC), pheophorbide, dibromofluorescein (DBF), IR700DX, naphthalocyanine, and porphyrin derivatives.

In some embodiments, the cargo molecule comprises a drug.

In some embodiments, the drug is selected from the group consisting of: an antihistamine, a GABA receptor modulator, a neurotransmitter reuptake inhibitor, a local anesthetic, an anticholinergic, a sodium channel blocker, a calcium channel blocker, a thyrotropin-releasing hormone, a γ-secretase inhibitor, an AMPA receptor agonist or antagonist, an NMDA receptor agonist or antagonist, an mGlu receptor agonist or antagonist, a growth factor, an antiemetic agent, a corticosteroid, a nonsteroidal anti-inflammatory drug (NSAID), an anti-epileptic agent, a neurotropic agent, a cytotoxic agent, an antioxidant, an iron chelator, a mitochondrial modulator, a sirtuin modulator, a nitric oxide (NO) and/or nitric oxide synthase (NOS) modulator, a potassium channel agonist or antagonist, a purigenic receptor agonist or antagonist, or any combination thereof.

In some embodiments, the drug is selected from the group consisting of: benzocaine; carticaine; cinchocaine; cyclomethycaine; lidocaine; prilocaine; propxycaine; proparacaine; tetracaine; tocainide; trimecaine; methotrexate; cyclophosphamide; thalidomide; paclitaxel; pemetrexed; pentostatin; pipobroman; pixantrone; plicamycin; platonin; procarbazine; raltitrexed; rebeccamycin; rubitecan; SN-38; salinosporamide A; satraplatin; streptozotocin; swainsonine; tariquidar; taxane; tegafur-uracil; temozolomide; testolactone; thioTEPA; tioguanine; topotecan; trabectedin; tretinoin; triplatin tetranitrate; tris(2-chloroethyl)amine; troxacitabine; uracil mustard; valrubicin; vinblastine; vincristine; vinorelbine; vorinostat; zosuquidar; carbamazepine; oxcarbazepine; phenytein; valproic acid; sodium valproate; cinnarizine; flunarizine; nimodipine; brain-derived neurotrophic factor (BDNF); ciliary neurotrophic factor (CNTF); glial cell-line derived neurotrophic factor (GDNF); neurotrophin-3; neurotrophin-4; fibroblast growth factor (FGF) receptor; insulin-like growth factor (IGF); prednisone; prednisolone; dexamethasone; gabapentin; or any combination thereof.

In another aspect, the present disclosure provides a pharmaceutical composition comprising a nerve targeting peptide conjugate described herein and a pharmaceutically acceptable carrier.

In yet another aspect, the present disclosure provides a method of delivering a cargo molecule to a neuron or nerve comprising contacting the neuron or nerve with a nerve targeting peptide conjugate of the present disclosure.

In yet another aspect, the present disclosure provides a method of identifying a neuron or nerve comprising contacting the neuron or nerve with a nerve targeting peptide conjugate of the present disclosure.

In yet another aspect, the present disclosure provides a method of delivering a drug to a neuron or nerve comprising contacting the neuron or nerve with a nerve targeting peptide conjugate of the present disclosure.

In another aspect, the present disclosure provides a method of delivering a photosensitizing agent to a neuron or nerve comprising contacting the neuron or nerve with a nerve targeting peptide conjugate of the present disclosure.

### DETAILED DESCRIPTION

Phage display screening was used to identify human nerve binding peptide sequences specific to proteins expressed in the human peripheral nerve (e.g., nidogen-2, myelin protein zero (MPZ), myelin proteolipid protein (myelin PLP), and myelin basic protein), as well as specific laminin trimers expressed in human nerves. Peptides described herein may bind to motor, sensory and autonomic nerves. Nerve targeting peptides can be used for nerve targeting peptide conjugates comprising a nerve targeting peptide comprising the amino acid sequence of any one of SEQ ID NOS: 1-710; and a cargo molecule, such as a drug, fluorescent moiety, or photosensitizing agent. Nerve targeting peptide conjugates of the present disclosure may be used for delivering a cargo molecule to a neuron or nerve, identifying a neuron or nerve, delivering a drug to a neuron or nerve, or delivering a photosensitizing agent to a neuron or nerve.

The invention may be more fully appreciated by reference to the following description, including the examples. Unless otherwise defined, all technical and scientific terms used herein have the same meaning as commonly understood by one of ordinary skill in the art. The use of headings and subheadings provided in the sections of this specification is solely for convenience of reference and does not limit the various embodiments herein, which are to be construed by reference to the specification as a whole.

Prior to setting forth this disclosure in more detail, it may be helpful to an understanding thereof to provide definitions of certain terms to be used herein. Additional definitions are set forth throughout this disclosure.

The term “consisting essentially of” limits the scope of a claim to the specified materials or steps and those that do not materially affect the basic and novel characteristics of the claimed invention. It should be understood that the terms “a” and “an” as used herein refer to “one or more” of the enumerated components. The use of the alternative (e.g., “or”) should be understood to mean either one, both, or any combination thereof of the alternatives. As used herein, the terms “include” and “have” are used synonymously, which terms and variants thereof are intended to be construed as non-limiting. The term “comprise” means the presence of the stated features, integers, steps, or components as referred to in the claims, but that it does not preclude the presence or addition of one or more other features, integers, steps, components, or groups thereof.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means±20% of the indicated range, value, or structure, unless otherwise indicated.

In addition, it should be understood that the individual compounds, or groups of compounds, derived from the various combinations of the structures and substituents described herein, are disclosed by the present application to the same extent as if each compound or group of compounds was set forth individually. Thus, selection of particular structures or particular substituents is within the scope of the present disclosure

As used herein, the “central nervous system” or “CNS” refers to the brain and the spinal cord, as well as the retina.

As used herein, the “peripheral nervous system” or “PNS” refers to the nerves and ganglia outside the CNS. The PNS is divided into the somatic nervous system and the autonomic nervous system. The somatic nervous system controls all voluntary muscular systems within the body, and the process of voluntary reflex arcs and is composed of afferent or sensory nerves, which relay sensation from the body to the central nervous system, and efferent or motor nerves, which send out commands from the CNS to the body, stimulating muscle contraction. The autonomic nervous system supplies smooth muscle and glands, and thus influences the function of internal organs. The autonomic nervous system acts largely unconsciously and regulates bodily functions, such as the heart rate, digestion, respiratory rate, pupillary response, urination, and sexual arousal.

As used herein, a “neuron” refers to an electrically excitable cell that processes and transmits information by electrical and chemical signaling. A typical neuron possesses a cell body (often called the soma), dendrites, and an axon.

As used herein, a “nerve” refers to an enclosed, cable-like bundle of neural axons. Each nerve is a cordlike structure that contains many axons. Each axon within the nerve is an extension of an individual neuron. Within a nerve, each axon is surrounded by a layer of connective tissue called the endoneurium. The axons are bundled together into groups called fascicles, and each fascicle is wrapped in a layer of connective tissue called the perineurium. Each nerve is covered on the outside by a dense sheath of connective tissue, the epineurium.

As used herein the term “conjugate” refers to a peptide attached to at least one cargo molecule, optionally via a linker.

The terms “polypeptide,” “peptide,” and “protein” are used interchangeably herein to refer to a polymer of amino acid residues. The terms apply to naturally occurring amino acid polymers as well as amino acid polymers in which one or more amino acid residues is a non-naturally occurring amino acid (e.g., an amino acid analog). The terms encompass amino acid chains of any length, including full length proteins (i.e., antigens), wherein the amino acid residues are linked by covalent peptide bonds. As used herein, the term “peptide” refers to a polymer of amino acid residues typically ranging in length from 2 to about 50 residues. Where an amino acid sequence is provided herein, L-, D-, or beta amino acid versions of the sequence are also contemplated as well as retro, inversion, and retro-inversion isoforms. Peptides also include amino acid polymers in which one or more amino acid residues is an artificial chemical analogue of a corresponding naturally occurring amino acid, as well as to naturally occurring amino acid polymers. In addition, the term applies to amino acids joined by a peptide linkage or by other modified linkages (e.g., where the peptide bond is replaced by an α-ester, a /3-ester, a thioamide, phosphonamide, carbamate, hydroxylate, and the like (see, e.g., Spatola, (1983) Chem. Biochem. Amino Acids and Proteins 7: 267-357), where the amide is replaced with a saturated amine (see, e.g., Skiles et al., U.S. Pat. No. 4,496,542, which is incorporated herein by reference, and Kaltenbronn et al., (1990) pp. 969-970 in Proc. 11th American Peptide Symposium, ESCOM Science Publishers, The Netherlands, and the like)).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. An amino acid may be an L- or D-amino acid. Amino acid analogs refer to compounds that have the same basic chemical structure as a naturally occurring amino acid, i.e., a carbon that is bound to a hydrogen, a carboxyl group, an amino group, and an R group, e.g., homoserine, norleucine, methionine sulfoxide. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

Amino acids may be referred to herein by either their commonly known three letter symbols or by the one-letter symbols recommended by the IUP AC-IUB Biochemical Nomenclature Commission. Nucleotides, likewise, may be referred to by their commonly accepted single-letter codes.

One of skill will recognize that individual substitutions, deletions or additions to a peptide, polypeptide, or protein sequence which alter, add or delete a single amino acid or a small percentage of amino acids in the encoded sequence represent a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the present disclosure.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

“Sequence identity,” as used herein, refers to the percentage of amino acid residues in a single sequence that are identical with the amino acid residues in another reference polypeptide sequence after aligning the sequences and introducing gaps, if necessary, to achieve the maximum percent sequence identity, and not considering any conservative substitutions as part of the sequence identity. The percentage sequence identity values can be generated using the NCBI BLAST 2.0 software as defined by Altschul et al. (1997), Nucl. Acids Res. 25:3389-3402, with the parameters set to default values.

As used herein, the terms “label” refers to a molecule that facilitates the visualization and/or detection of a targeting molecule disclosed herein. In some embodiments, the label is a fluorescent moiety.

The phrase “specifically binds” when referring to the interaction between a peptide disclosed herein and a target (e.g., purified protein, neuron or nerve tissue, neuron or nerves, cranial neuron or nerves, central neuron or nerves, myelinated or unmyelinated neuron or nerves, or connective tissue surrounding neuron or nerves), refers to the formation of a high affinity bond between the targeting molecule and the target. Further, the term means that the peptide has low affinity for non-targets.

“Selective binding,” “selectivity,” and the like refer to the preference of agent to interact with one molecule as compared to another. Preferably, interactions between a peptide disclosed herein and a target are both specific and selective. Note that in some embodiments an agent is designed to “specifically bind” and “selectively bind” two distinct, yet similar targets without binding to other undesirable targets.

The terms “individual,” “patient,” or “subject” are used interchangeably. As used herein, they mean any mammal (i.e. species of any orders, families, and genus within the taxonomic classification animalia: chordata: vertebrata: mammalia). In some embodiments, the mammal is a cow, horse, sheep, pig, cat, dog, goat, mouse, rat, rabbit, guinea pig, non-human primate, or human. None of the terms require or are limited to a situation characterized by the supervision (e.g. constant or intermittent) of a health care worker (e.g. a doctor, a registered nurse, a nurse practitioner, a physician's assistant, an orderly, or a hospice worker).

The terms “administer,” “administering”, “administration,” and the like, as used herein, refer to the methods that may be used to enable delivery of agents or compositions to the desired site of biological action. These methods include, but are not limited to topical, oral, intrarectal, intravaginal, intranasal, inhalation, parenteral injection (e.g., intravenous, intraarterial, subcutaneous, intraperitoneal, intramuscular, intravascular, intrathecal, intravitreal, infusion, or local). Administration techniques that are optionally employed with the agents and methods described herein, include e.g., as discussed in Goodman and Gilman, The Pharmacological Basis of Therapeutics, current ed.; Pergamon; and Remington's, Pharmaceutical Sciences (current edition), Mack Publishing Co., Easton, Pa. Administration of agents may be local or systemic. In some embodiments, administration is via systemic intravenous injection into human patients.

The term “pharmaceutically acceptable” as used herein, refers to a material that does not abrogate the biological activity or properties of the agents described herein, and is relatively nontoxic (i.e., the toxicity of the material significantly outweighs the benefit of the material). In some instances, a pharmaceutically acceptable material may be administered to an individual without causing significant undesirable biological effects or significantly interacting in a deleterious manner with any of the components of the composition in which it is contained.

As used herein, “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material, and having the properties of being nontoxic and non-inflammatory in a patient. Each excipient must be “acceptable” in the sense of being compatible with the other ingredients of the formulation and not injurious to the patient.

Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, or waters of hydration. Examples of pharmaceutical excipients can be sterile liquids, such as water and oils, including those of petroleum, animal, vegetable or synthetic origin, such as peanut oil, soybean oil, mineral oil, sesame oil and the like. Water or aqueous solutions, saline solutions, and aqueous dextrose and glycerol solutions are preferably employed as excipients, particularly for injectable solutions. Suitable pharmaceutical excipients are described in “Remington's Pharmaceutical Sciences” by E. W. Martin.

The term “surgery” as used herein, refers to any methods for that may be used to manipulate, change, or cause an effect by a physical intervention. These methods include, but are not limited to open surgery, endoscopic surgery, laparoscopic surgery, minimally invasive surgery, robotic surgery, any procedures that may affect any neuron or nerves such as placement of retractors during spinal surgery, microscopic procedures, cardiac neuron or nerve ablation, epidural injection, intrathecal injections, neuron or nerve blocks, implantation of devices such as neuron or nerve stimulators and implantation of pumps. In some embodiments, the subject of the surgery is a human subject or human patient.

### I. Peptides

In one aspect, the present disclosure provides peptides that bind to proteins expressed in the human peripheral nerve that can be used in nerve targeting peptide conjugates of the present disclosure. In some embodiments, the peptide ranges in length from about 5 residues to about 50 residues, about 6 residues to about 40 residues, about 7 residues to about 30 residues, about 7 residues to about 20 residues, or about 7 residues to about 15 residues. In some embodiments, the peptide ranges in length from about 5, 6, 7, 8, 9, 10, 11, 12, 13, 14, 15, 16, 17, 18, 19, or 20 residues to about 50, 45, 40, 35, 30, or 25 residues. In some embodiments, the peptide ranges in length from about 7, 8, 9, 10, 11, or 12 residues to about 15, 20, 25 or 30 residues.

In some embodiments, the peptide comprises or consists or the amino acid sequence set forth in any one of SEQ ID NOS:1-66. In some embodiments, the peptide comprises or consists of the amino acid sequence set forth in any one of SEQ ID NOS:1-20. In some embodiments, the peptide comprises or consists of the amino acid sequence set forth in any one of SEQ ID NOS:21-40. In some embodiments, the peptide comprises or consists of the amino acid sequence set forth in any one of SEQ ID NOS:41-60. In some embodiments, the peptide comprises or consists of the amino acid sequence set forth in any one of SEQ ID NOS:61-66.

In some embodiments, the peptide comprises or consists or the amino acid sequence set forth in any one of SEQ ID NOS: 1-710. In some embodiments, the peptide comprises or consists of the amino acid sequence set forth in any one of SEQ ID NOS:1-20 and 67-151 (MPZ binding peptides). In some embodiments, the peptide comprises or consists of the amino acid sequence set forth in any one of SEQ ID NOS:21-40 and 152-326 (MBP binding peptides). In some embodiments, the peptide comprises or consists of the amino acid sequence set forth in any one of SEQ ID NOS:41-60 and 327-410 (PLP binding peptides). In some embodiments, the peptide comprises or consists of the amino acid sequence set forth in any one of SEQ ID NOS:61-66 and 411-435 (NID-2 binding peptides). In some embodiments, the peptide comprises or consists of the amino acid sequence set forth in any one of SEQ ID NOS:436-642 (Laminin 421/521 binding peptides). In some embodiments, the peptide comprises or consists of the amino acid sequence set forth in any one of SEQ ID NOS:643-710 (Nerve tissue/extract binding peptides).

In some embodiments, the peptide comprises or consists or the amino acid sequence set forth in any one of SEQ ID NOS: 1, 2, 4-17, 23, 24, 29, 31, 34, 35, 37, 40, 41, 42, 44-47, 49, 50, 52-60, and 67-710.

In some embodiments, the peptide comprises or consists of the amino acid sequence set forth in any one of SEQ ID NOS: 1, 2, 4-17 and 67-151.

In some embodiments, the peptide comprises or consists of the amino acid sequence set forth in any one of SEQ ID NOS:23, 24, 29, 31, 34, 35, 37, 40, and 152-326.

In some embodiments, the peptide comprises or consists of the amino acid sequence set forth in any one of SEQ ID NOS: 41, 42, 44-47, 49, 50, 52-60, and 327-410.

In some embodiments, the peptide comprises or consists of the amino acid sequence set forth in any one of SEQ ID NOS:411-435.

In some embodiments, the peptide comprises the amino acid sequence having at least 75% identity to any one of SEQ ID NOS:1-66. In some embodiments, the peptide comprises the amino acid sequence having at least 80% identity to any one of SEQ ID NOS:1-66. In some embodiments, the peptide comprises the amino acid sequence having at least 85% identity to any one of SEQ ID NOS:1-66. In some embodiments, the peptide comprises the amino acid sequence having at least 90% identity to any one of SEQ ID NOS:1-66. In some embodiments, the peptide comprises the amino acid sequence having at least 95% identity to any one of SEQ ID NOS: 1-66. In some embodiments, the peptide comprises the amino acid sequence having at least 97% identity to any one of SEQ ID NOS: 1-66.

In some embodiments, the peptide comprises the amino acid sequence having at least 75% identity to any one of SEQ ID NOS: 1-710. In some embodiments, the peptide comprises the amino acid sequence having at least 80% identity to any one of SEQ ID NOS:1-710. In some embodiments, the peptide comprises the amino acid sequence having at least 85% identity to any one of SEQ ID NOS: 1-710. In some embodiments, the peptide comprises the amino acid sequence having at least 90% identity to any one of SEQ ID NOS: 1-710. In some embodiments, the peptide comprises the amino acid sequence having at least 95% identity to any one of SEQ ID NOS:1-710. In some embodiments, the peptide comprises the amino acid sequence having at least 97% identity to any one of SEQ ID NOS: 1-710.

In some embodiments, the peptide comprises the amino acid sequence having at least 75% identity to any one of SEQ ID NOS: 1, 2, 4-17, 23, 24, 29, 31, 34, 35, 37, 40, 41, 42, 44-47, 49, 50, 52-60, and 67-710. In some embodiments, the peptide comprises the amino acid sequence having at least 80% identity to any one of SEQ ID NOS: 1, 2, 4-17, 23, 24, 29, 31, 34, 35, 37, 40, 41, 42, 44-47, 49, 50, 52-60, and 67-710. In some embodiments, the peptide comprises the amino acid sequence having at least 85% identity to any one of SEQ ID NOS: 1, 2, 4-17, 23, 24, 29, 31, 34, 35, 37, 40, 41, 42, 44-47, 49, 50, 52-60, and 67-710. In some embodiments, the peptide comprises the amino acid sequence having at least 90% identity to any one of SEQ ID NOS:1, 2, 4-17, 23, 24, 29, 31, 34, 35, 37, 40, 41, 42, 44-47, 49, 50, 52-60, and 67-710. In some embodiments, the peptide comprises the amino acid sequence having at least 95% identity to any one of SEQ ID NOS: 1, 2, 4-17, 23, 24, 29, 31, 34, 35, 37, 40, 41, 42, 44-47, 49, 50, 52-60, and 67-710. In some embodiments, the peptide comprises the amino acid sequence having at least 97% identity to any one of SEQ ID NOS:1, 2, 4-17, 23, 24, 29, 31, 34, 35, 37, 40, 41, 42, 44-47, 49, 50, 52-60, and 67-710.

In some embodiments, peptides of the present disclosure are acetylated at the N-terminus (“Ac” or “acetyl”), amidated at the C-terminus (“CONH₂” or “NH₂”), or both.

The peptides of the present disclosure may be synthesized by any suitable method. In some embodiments, the peptides are chemically synthesized, e.g., by solid phase peptide synthesis. Techniques for solid phase synthesis are described, for example, by Barany and Merrifield (1963) Solid-Phase Peptide Synthesis; pp. 3-284 in The Peptides: Analysis, Synthesis, Biology. Vol. 2: Special Methods in Peptide Synthesis, Part A.; Merrifield et al. (1963) J. Am. Chem. Soc, 85: 2149-2156, and Stewart et al. (1984) Solid Phase Peptide Synthesis, 2nd ed. Pierce Chem. Co., Rockford, 111. In some embodiments, the peptides are recombinantly produced via cell-based expression systems. A polynucleotide encoding a peptide of the present disclosure may be incorporated into a recombinant expression vector for production of the peptide in a host cell. The expression vector also comprises expression control sequences, such as a promoter, enhancer, initiation site, and the like. The polynucleotide encoding the peptide is operatively linked to at least one expression control sequence in the expression vector to direct mRNA synthesis. Host cells may be genetically engineered (transduced, transformed, or transfected) with the vectors. The vector may be in the form of a plasmid, a viral particle, a phage, etc. The engineered host cells can be cultured in appropriate media and under suitable conditions, such as temperature, pH, and the like, and for a suitable time to express a sufficient amount of the peptide. In some embodiments, the synthesized peptides are purified using methods such as preparative reversed phase chromatography, partition chromatography, gel filtration, gel electrophoresis, or ion-exchange chromatography or other methods used in the art.

### II. Cargo Molecule

In some embodiments, the peptide of a nerve targeting peptide conjugate is directly bound to a cargo molecule. In some embodiments, the peptide of a nerve targeting peptide conjugate is indirectly (e.g., via a linker) bound to a cargo molecule. In some embodiments, the peptide is bound to a cargo molecule at its N-terminus, at its C-terminus, or at an internal position (e.g., to an internal amino acid) of the peptide. In some embodiments, two, three, four or more peptides are directly or indirectly bound to a cargo molecule.

In certain embodiments, a cargo molecule comprises a drug, fluorescent moiety, photosensitizing agent, radiolabel, neurotrophic factor, or a combination thereof. In some embodiments, the cargo molecule comprises a drug. In some embodiments, the cargo molecule comprises a fluorescent moiety. In some embodiments, the cargo molecule comprises a photosensitizing agent. In some embodiments, the peptide is bound to two or more cargo molecules. The two or more cargo molecule may be the same molecule or different molecules, or be from the same class of cargo molecule (e.g., two drugs) or from different classes of cargo molecules (e.g., one drug and one fluorescent moiety).

In some embodiments, the peptide comprises or consists or the amino acid sequence set forth in any one of SEQ ID NOS: 1-66. In some embodiments, the peptide comprises or consists of the amino acid sequence set forth in any one of SEQ ID NOS:1-20. In some embodiments, the peptide comprises or consists of the amino acid sequence set forth in any one of SEQ ID NOS:21-40. In some embodiments, the peptide comprises or consists of the amino acid sequence set forth in any one of SEQ ID NOS:41-60. In some embodiments, the peptide comprises or consists of the amino acid sequence set forth in any one of SEQ ID NOS:61-66.

In some embodiments, the peptide comprises or consists or the amino acid sequence set forth in any one of SEQ ID NOS: 1-710. In some embodiments, the peptide comprises or consists of the amino acid sequence set forth in any one of SEQ ID NOS:1-20 and 67-151. In some embodiments, the peptide comprises or consists of the amino acid sequence set forth in any one of SEQ ID NOS:21-40 and 152-326. In some embodiments, the peptide comprises or consists of the amino acid sequence set forth in any one of SEQ ID NOS:41-60 and 327-410. In some embodiments, the peptide comprises or consists of the amino acid sequence set forth in any one of SEQ ID NOS:61-66 and 411-435. In some embodiments, the peptide comprises or consists of the amino acid sequence set forth in any one of SEQ ID NOS:436-642. In some embodiments, the peptide comprises or consists of the amino acid sequence set forth in any one of SEQ ID NOS:643-710.

In some embodiments, the peptide comprises or consists or the amino acid sequence set forth in any one of SEQ ID NOS: 1, 2, 4-17, 23, 24, 29, 31, 34, 35, 37, 40, 41, 42, 44-47, 49, 50, 52-60, and 67-710.

In some embodiments, the peptide comprises or consists of the amino acid sequence set forth in any one of SEQ ID NOS: 1, 2, 4-17 and 67-151.

In some embodiments, the peptide comprises or consists of the amino acid sequence set forth in any one of SEQ ID NOS:23, 24, 29, 31, 34, 35, 37, 40, and 152-326.

In some embodiments, the peptide comprises or consists of the amino acid sequence set forth in any one of SEQ ID NOS: 41, 42, 44-47, 49, 50, 52-60, and 327-410.

In some embodiments, the peptide comprises or consists of the amino acid sequence set forth in any one of SEQ ID NOS: 411-435.

### Fluorescent Moieties

In some embodiments, the cargo molecule of the nerve targeting peptide conjugate comprises a fluorescent moiety (e.g., a fluorescent protein, fluorescent peptide, or fluorophore (fluorescent dye) molecule). All fluorescent moieties are encompassed within the term “fluorescent moiety.” Specific examples of fluorescent moieties given herein, are illustrative and are not meant to limit the fluorescent moieties for use with the targeting molecules disclosed herein. Common classes of fluorophores (fluorescent dyes) include, but are not limited to, xanthenes, such as rhodamines, rhodols and fluoresceins, and their derivatives; bimanes; coumarins and their derivatives such as umbelliferone and aminomethyl coumarins; aromatic amines such as dansyl; squarate dyes; benzofurans; fluorescent cyanines; carbazoles; dicyanomethylene pyranes; polymethine; oxabenzanthrane; xanthene; pyrylium; carbostyl; perylene; acridone; quinacridone; rubrene; anthracene; coronene; phenanthrecene; pyrene; butadiene; stilbene; porphyrin; pthalocyanine; lanthanide metal chelate complexes; rare-earth metal chelate complexes; and derivatives of such dyes. Fluorescent dyes are discussed, for example, in U.S. Pat. Nos. 4,452,720; 5,227,487; and 5,543,295.

In some embodiments, the fluorescent moiety is selected from the group consisting of a xanthene; a bimane; a coumarin; an aromatic amines; a benzofuran; a fluorescent cyanine; a carbazole; a dicyanomethylene pyrane; polymethine; oxabenzanthrane; pyrylium; carbostyl; perylene; acridone; quinacridone; rubrene; anthracene; coronene; phenanthrecene; pyrene; butadiene; stilbene; porphyrin; pthalocyanine; lanthanide metal chelate complexes; rare-earth metal chelate complexes; FITC; Cy3; EGFP; cyan fluorescent protein (CFP); EGFP; 5-FAM; 6-FAM; FAM; fluorescein, IAEDANS, EDANS and BODIPY FL; TRITC; Cy5; Cy3; YFP; 6-FAM; LC Red 640; Alexa Fluor 546; fluorescein; tetramethylrhodamine; Dabcyl; BODIPY FL; QSY 7, QSY 9, QSY 21 and BBQ-650 dyes.

In some embodiments, the cargo molecule comprises a fluorescein dye. Typical fluorescein dyes include, but are not limited to, 5-carboxyfluorescein, fluorescein-5-isothiocyanate, 5(6)-carboxyfluorescein, 5,6-dicarboxyfluorescein, 5-(and 6)-sulfofluorescein, sulfonefluorescein, succinyl fluorescein, 5-(and 6)-carboxy SNARF-1, carboxyfluorescein sulfonate, carboxyfluorescein zwitterion, carboxyfluorescein quaternary ammonium, carboxyfluorescein phosphonate, carboxyfluorescein GABA, carboxyfluorescein-cys-Cy5, 5′(6′)-carboxyfluorescein, fluorescein glutathione, and 6-carboxyfluorescein; examples of other fluorescein dyes can be found, for example, in U.S. Pat. Nos. 6,008,379, 5,750,409, 5,066,580, and 4,439,356.

In some embodiments, the cargo molecule comprises a rhodamine dye, such as, for example, 5-(and 6)-carboxy rhodamine 110, tetramethylrhodamine-6-isothiocyanate, 5-carboxytetramethylrhodamine, 5-carboxy rhodol derivatives, tetramethyl and tetraethyl rhodamine, diphenyldimethyl and diphenyldiethyl rhodamine, dinaphthyl rhodamine, rhodamine 101 sulfonyl chloride (sold under the tradename of TEXAS RED®), and other rhodamine dyes. Other rhodamine dyes can be found, for example, in U.S. Pat. Nos. 6,080,852; 6,025,505; 5,936,087; 5,750,409. In some embodiments, a cargo moiety includes a cyanine dye, such as, for example, Cy3, Cy3B, Cy3.5, Cy5, Cy5.5, Cy 7, and indocynaine green.

In some embodiments, the fluorophore exhibits green fluorescence (such as for example 494 nm/519 nm), orange fluorescence (such as for example 554 nm/570 nm), red fluorescence (such as for example 590 nm/617 nm), or far red fluorescence (such as for example 651 nm/672 nm) excitation/emission spectra. In some embodiments, the fluorophore is a fluorophore with excitation and emission spectra in the range of about 350 nm to about 775 nm. In some embodiments the excitation and emission spectra are about 346 nm/446 nm, about 494 nm/519 nm, about 554 nm/570 nm, about 555 nm/572 nm, about 590 nm/617 nm, about 651 nm/672 nm, about 679 nm/702 nm, or about 749 nm/775 nm.

In some embodiments, the fluorophore can include but is not limited to AlexaFluor 3, AlexaFluor 5, AlexaFluor 350, AlexaFluor 405, AlexaFluor 430, AlexaFluor 488, AlexaFluor 500, AlexaFluor 514, AlexaFluor 532, AlexaFluor 546, AlexaFluor 555, AlexaFluor 568, AlexaFluor 594, AlexaFluor 610, AlexaFluor 633, AlexaFluor 647, AlexaFluor 660, AlexaFluor 680, AlexaFluor 700, and AlexaFluor 750 (Molecular Probes AlexaFluor dyes, available from Life Technologies, Inc. (USA)). In some embodiments, the fluorophore can include but is not limited to Cy dyes, including Cy2, Cy3, Cy3B, Cy3.5, Cy5, Cy5.5 and Cy7 (available from GE Life Sciences or Lumiprobes). In some embodiments the fluorophore can include but is not limited to DyLight 350, DyLight 405, DyLight 488, DyLight 550, DyLight 594, DyLight 633, DyLight 650, DyLight 680, DyLight 750 and DyLight 800 (available from Thermo Scientific (USA)). In some embodiments, the fluorophore can include but is not limited to a FluoProbes 390, FluoProbes 488, FluoProbes 532, FluoProbes 547H, FluoProbes 594, FluoProbes 647H, FluoProbes 682, FluoProbes 752 and FluoProbes 782, AMCA, DEAC (7-Diethylaminocoumarin-3-carboxylic acid); 7-Hydroxy-4-methylcoumarin-3; 7-Hydroxycoumarin-3; MCA (7-Methoxycoumarin-4-acetic acid); 7-Methoxycoumarin-3; AMF (4′-(Aminomethyl)fluorescein); 5-DTAF (5-(4,6-Dichlorotriazinyl)aminofluorescein); 6-DTAF (6-(4,6-Dichlorotriazinyl)aminofluorescein); FAM; 6-FAM (6-Carboxyfluorescein), 5(6)-FAM cadaverine; 5-FAM cadaverine; 5(6)-FAM ethylenediamme; 5-FAM ethylenediamme; 5-FITC (FITC Isomer I; fluorescein-5-isothiocyanate); 5-FITC cadaverin; Fluorescein-5-maleimide; 5-IAF (5-Iodoacetamidofluorescein); 6-JOE (6-Carboxy-4′,5′-dichloro-2′,7′-dimethoxyfluorescein); 5-CR1 10 (5-Carboxyrhodamine 110); 6-CR1 10 (6-Carboxyrhodamine 110); 5-CR6G (5-Carboxyrhodamine 6G); 6-CR6G (6-Carboxyrhodamine 6G); 5(6)-Carboxyrhodamine 6G cadaverine; 5(6)-Caroxyrhodamine 6G ethylenediamme; 5-ROX (5-Carboxy-X-rhodamine); 6-ROX (6-Carboxy-X-rhodamine); 5-TAMRA (5-Carboxytetramethylrhodamine); 6-TAMRA (6-Carboxytetramethylrhodamine); 5-TAMRA cadaverine; 6-TAMRA cadaverine; 5-TAMRA ethylenediamme; 6-TAMRA ethylenediamme; 5-TMR C6 maleimide; 6-TMR C6 maleimide; TR C2 maleimide; TR cadaverine; 5-TRITC; G isomer (Tetramethylrhodamine-5-isothiocyanate); 6-TRITC; R isomer (Tetramethylrhodamine-6-isothiocyanate); Dansyl cadaverine (5-Dimethylaminonaphthalene-1-(N-(5-aminopentyl))sulfonamide); EDANS C2 maleimide; fluorescamine; NBD; and pyrromethene and derivatives thereof.

In some embodiments, a cargo comprises an environmentally sensitive fluorescent dye or fluorophore. Examples of environmentally sensitive fluorescent dyes or fluorophores include 5,6-carboxy-diethyl rhodol (pH sensitive), merocyanine (membrane potential sensitive), and Nile red carboxylic acid (lipid sensitive).

In some embodiments, the fluorescent moiety is a peptide or protein. In some embodiments, the fluorescent moiety is Green Fluorescent Protein (GFP). In some embodiments, the fluorescent moiety is a derivative or variant of GFP, including for example: EGFP, emerald, superfolder GFP, azami green mWasabi, TagGFP, TurboGFP, AcGFP, ZsGreen, T-Sapphire, EBFP, EBFP2, Azurite, mTagBFP, ECFP, mECFP, Cerulean, mTurquoise, CyPet, AmCyan1, Midori-Ishi Cyan, TagCFP, mTFP1 (Teal), EYFP, Topaz, Venus, mCitrine, YPet, TagYFP, PhiYFP, ZsYellow1, mBanana, Kusabira Orange, Kusbira Orange2, mOrange, mOrange2, dTomato, dTomato-Tandem, TagRFP, TagRFP-Y, DsRed, DsRed2, DsRed-Express (T1), DsRed-Monomer, mTangerine, mRuby, mApple, mStrawberry, AsRed2, mRFP1, JRed, mCherry, HcRed1, mRaspberry, dKeima-Tandem, HcRed-Tandem, mPlum, AQ143, mKalamal, YFP, and Citrine.

Fluorescent moieties are detected by any suitable method. For example, a fluorescent moiety may be detected by exciting the fluorochrome with the appropriate wavelength of light and detecting the resulting fluorescence, e.g., by microscopy, visual inspection, via photographic film, by the use of electronic detectors such as charge coupled devices (CCDs), photomultipliers, etc.

In some embodiments, the fluorescent moiety is conjugated to high molecular weight molecule, such as water soluble polymers including, but not limited to, dextran, PEG, serum albumin, or poly(amidoamine) dendrimer.

### Photosensitizing Agents

In some embodiments, a cargo molecule of a nerve targeting peptide conjugate comprises a photosensitizing agent. A photosensitizing agent is any agent or compound useful in light induced ablation therapy. Such agents, when exposed to a specific wavelength of light, react with molecular oxygen to produce singlet oxygen, which is highly cytotoxic. Thus, targeting molecules of the present invention comprising a photosensitizing agent may be used to focally injure nerves. In certain embodiments, a photosensitizing agent is a porphyrin, chlorin, or dye. Examples of photosensitizing agents include porphyrin, protoporfin IX, purlytin, verteporfin, HPPH, temoporfin, methylene blue, photofrin, protofrin, hematoporphyrin, Talaporfin, benzopophyrin derivative monoacid, 5-aminileuvolinic acid, Lutetium texaphyrin, metallophthalocyanine, metallo-naphthocyaninesulfobenzo-porphyrazines, metallo-naphthalocyanines, zinc tetrasulfophthalocyanine, bacteriochlorins, metallochlorins, chlorine derivative, Tetra(m-hydroxyphenyl)chlorin (mTHPC), pheophorbide, dibromofluorescein (DBF), IR700DX, naphthalocyanine, and porphyrin derivatives.

In some embodiments, the photosensitizing agent is conjugated to a C-terminal cysteine residue of the human neuron or nerve targeting molecule via maleimide mediated conjugation. Preferably, the photosensitizing agent of the present disclosure is activated by light having a wavelength of between 400 nm to 700 nm. Still more preferably, the photosensitizing agent in the present disclosure is activated at 627 nm and 660 nm. An optimal light dose can be identified to generate maximal nerve killing with minimal injury to adjacent tissue.

### Drugs

In some embodiments, a cargo molecule of a nerve targeting peptide conjugate comprises a drug. All drugs that act on a neuron or nerve (or a component thereof) are encompassed within the term “drug.” Specific examples of drug given herein, are illustrative and are not meant to limit the drugs for use with the targeting molecules disclosed herein.

In some embodiments, the drug is selected from a drug that: induces cell death (apoptotic or necrotic), inhibits cell death (apoptotic or necrotic), inhibits the transmission of a neuron or nerve signal (i.e., an electrochemical impulse), inhibits the release of a neurotransmitter, agonizes the activity of a gamma-aminobutyric acid (GABA) receptor, partially or fully inhibits the repolarization of a neuron, disrupts the conduction of an ion channel, or a combination thereof.

In some embodiments, the drug is an antihistamine, a GABA receptor modulator, a neurotransmitter reuptake inhibitor, a local anesthetic, an anticholinergic, a sodium channel blocker, a calcium channel blocker, a thyrotropin-releasing hormone, a γ-secretase inhibitor, an α-amino-3-hydroxy-5-methyl-4-isoxazolepropionic acid (AMPA) receptor agonist or antagonist, an N-methyl-D-aspartate (NMDA) receptor agonist or antagonist, an mGlu receptor agonist or antagonist, a growth factor, an antiemetic agent, a corticosteroid; a cytotoxic agent; an antioxidant, an iron chelator, a mitochondrial modulator, a sirtuin modulator, a nitric oxide (NO) and/or nitric oxide synthase (NOS) modulator, a potassium channel agonist or antagonist, a purigenic receptor agonist or antagonist, or a combination thereof.

In some embodiments, the drug is meclizine, diphenhydramine, dimenhydrinate, loratadine, quetiapine, mepyramine, piperoxan, antazoline, carbinoxamine, doxylamine, clemastine, pheniramine, chlorphenamine, chlorpheniramine, dexchlorpheniramine, brompheniramine, triprolidine, cyclizine, chlorcyclizine, hydroxyzine, promethazine, alimemazine, trimeprazine, cyproheptadine, azatadine, ketotifen, oxatomide, meclizine hydrochloride, promethazine hydrochloride, cinnarizine, hydroxyzine pamoate, betahistine dihydrochloride, alprazolam, bromazepam, brotizolam, chlordiazepoxide, clonazepam, clorazepate, diazepam, estazolam, flunitrazepam, flurazepam, loprazolam, lorazepam, lormetazepam, idazolam, nimetazepam, nitrazepam, oxazepam, prazepam, temazepam, triazolam, clonazepam, diazepam, lorazepam, furosemide, bumetanide, ethacrynic acid, gabapentin, pregabalin, muscimol, baclofen, amitriptyline, nortriptyline, trimipramine, fluoxetine, paroxetine, sertraline, glycopyrrolate, homatropine, scopolamine, atropine, benzocaine, carticaine, cinchocaine, cyclomethycaine, lidocaine, prilocaine, propxycaine, proparacaine, tetracaine, tocainide, trimecaine, carbamazepine, oxcarbazepine, phenytein, valproic acid, sodium valproate, cinnarizine, flunarizine, nimodipine, thyrotropin-releasing hormone, amifostine (also known as WR-2721, or ETHYOL®); a carbamate compound (e.g., 2-phenyl-1,2-ethanediol monocarbomates and dicarbamates); LY450139 (hydroxylvaleryl monobenzocaprolactam); L685458 (1S-benzyl-4R[1-[1-S-carbamoyl-2-phenethylcarbamoyl)-1S-3-methylbutylcarbamoyl]-2R-hydroxy-5-phenylpentyl}carbamic acid tert-butyl ester); LY411575 (N2-[(2S)-2-(3,5-difluorophenyl)-2-hydroxyethanoyl]-N1[(7S)-5-methyl-6-oxo-6,7-dihydro-5H-dibenzo[bid]azepin-7yl]-L-alaninamide); MK-0752; tarenflurbil; BMS-299897 (2-[(1R)-1-[[(4-chlorophenyl) sulfony](2,5-difluorophenyl)amino]ethyl]-5-fluorobenzenepropanoic acid; CNQX (6-cyano-7-nitroquinoxaline-2,3-dione); NBQX (2,3-dihydroxy-6-nitro-7-sulfamoyl-benzo[f]quinoxaline-2,3-dione); DNQX (6,7-dinitroquinoxaline-2,3-dione); kynurenic acid; 2,3-dihydroxy-6-nitro-7-sulfamoylbenzo-[f]quinoxaline; 1-aminoadamantane; dextromethorphan; dextrorphan; ibogaine; ketamine; nitrous oxide; phencyclidine; riluzole; tiletamine; memantine; dizocilpine; aptiganel; remacimide; 7-chlorokynurenate; DCKA (5,7-dichlorokynurenic acid); kynurenic acid; 1-aminocyclopropanecarboxylic acid (ACPC); AP7 (2-amino-7-phosphonoheptanoic acid); APV (R-2-amino-5-phosphonopentanoate); CPPene (3-[(R)-2-carboxypiperazin-4-yl]-prop-2-enyl-1-phosphonic acid); (+)-(1S, 2S)-1-(4-hydroxy-phenyl)-2-(4-hydroxy-4-phenylpiperidino)-1-pro-panol; (1S, 2S)-1 -(4-hydroxy-3-methoxyphenyl)-2-(4-hydroxy-4-phenylpiperi-dino)-1-propanol; (3R, 4S)-3-(4-(4-fluorophenyl)-4-hydroxypiperidin-1-yl-)-chroman-4;7-diol; (IR*, 2R*)-1-(4-hydroxy-3-memylphenyl)-2-(4-(4-fluoro-phenyl)-4-hydroxypiperidin-1-yl)-propan-1-ol-mesylate); LY389795 ((−)-2-thia-4-aminobicyclo-hexane-4,6-dicarboxylate); LY379268 ((−)-2-oxa-4-aminobicyclo-hexane-4,6-dicarboxylate); LY354740 ((+)-2-aminobicyclo-hexane-2,6dicarboxylate); DCG-IV ((2S,2′R,3′R)-2-(2′,31-dicarboxycyclopropyl)glycine); 2R,4R-APDC (2R,4R-4-aminopyrrolidine-2,4-dicarboxylate); (S)-3C4HPG ((S)-3-carboxy-4-hydroxyphenylglycine); (S)-4C3HPG ((S)-4-carboxy-3-hydroxyphenylglycine); L-CCG-I ((2S, 1 ‘S,2′S)-2-(carboxycyclopropyl)glycine); ACPT-I ((1S,3R,4S)-1-aminocyclopentane-1,3,4-tricarboxylic acid); L-AP4 (L-(+)-2-Amino-4-phosphonobutyric acid); (S)-3,4-DCPG ((S)-3,4-dicarboxyphenylglycine); (RS)-3,4-DCPG ((RS)-3,4-dicarboxyphenylglycine); (RS)-4-phosphonophenylglycine ((RS)PPG); AMN082 (N,N’-bis(diphenylmethyl)-1,2-ethanediamine dihydrochloride); DCG-IV ((2S,2′R,3′R)-2-(2′,3′-dicarboxycyclopropyl)glycine); AMN082; brain-derived neurotrophic factor (BDNF); ciliary neurotrophic factor (CNTF); glial cell-line derived neurotrophic factor (GDNF); neurotrophin-3; neurotrophin-4; fibroblast growth factor (FGF) receptor; insulin-like growth factor (IGF); an aminoglycoside antibiotic (e.g., gentamicin and amikacin); a macrolide antibiotic (e.g, erythromycin); a glycopeptide antibiotic (e.g. vancomycin); salicylic acid; nicotine; Eburnamenine-14-carboxylic acid ethyl ester; sipatrigine (2-(4-Methylpiperazin-1-yl)-5-(2,3,5-trichlorophenyl)-pyrimidin-4-amine); amiloride (3,5-diamino-N-(aminoiminomethyl)-6-chloropyrazinecarbox amide hydrochloride); carbamazepine (5H-dibenzo[b,f]azepine-5-carboxamide); TTX (octahydro-12-(hydroxymethyl)-2-imino-5,9:7,10a-dimethan o-10aH-[1,3]dioxocino[6,5-d]pyrimidine-4,7,10,11,12-pen tol); RS100642 (1-(2,6-dimethyl-phenoxy)-2-ethylaminopropane hydrochloride); mexiletine ((1-(2,6-dimethylphenoxy)-2-aminopropane hydrochloride)); QX-314 (N-(2,6-Dimethylphenylcarbamoylmethyl)triethylammonium bromide); phenytoin (5,5-diphenylimidazolidine-2,4-dione); lamotrigine (6-(2,3-dichlorophenyl)-1,2,4-triazine-3,5-diamine); 4030W92 (2,4-diamino-5-(2,3-dichlorophenyl)-6-fluoromethylpyrimidine); BW1003C87 (5-(2,3,5-trichlorophenyl) pyrimidine-2,4-1.1 ethanesulphonate); QX-222 (2-[(2,6-dimethylphenyl)amino]-N,N,N-trimethyl-2-oxoetha niminium chloride); ambroxol (trans-4-[[(2-Amino-3,5 -dibromophenyl)methyl]amino]cyclo hexanol hydrochloride); R56865 (N-[1-(4-(4-fluorophenoxy)butyl]-4-piperidinyl-N-methyl-2-benzo-thiazolamine); lubeluzole; ajmaline ((17R,21alpha)-ajmalan-17,21-diol); procainamide (4-amno-N-(2-diethylaminoethyljbenzamide hydrochloride); flecainide; riluzoleor; triamicinolone actenoide; Dexamethasone; promethazine; prochlorperazine; trimethobenzamide; triethylperazine; dolasetron; granisetron; ondansetron; tropisetron; and palonosetron; droperidol; meclizine; perphenazine; thiethyl perazine; domperidone; properidol; haloperidol; chlorpromazine; promethazine; prochlorperazine; metoclopramide; dronabinol; nabilone; sativex; scopolamine; dexamethasone; trimethobenzamine; emetrol; propofol; muscimol; acridine carboxamide; actinomycin; 17-N-allylamino-17-demethoxygeldanamycin; amcinonide; amsacrine; aminopterin; anthracycline; antineoplastic; antineoplaston; 5-azacytidine; azathioprine; BL22; beclometasone; bendamustine; betamethasone; biricodar; bleomycin; bortezomib; bryostatin; budesonide; busulfan; calyculin; camptothecin; capecitabine; carboplatin; chlorambucil; cisplatin; cladribine; clofarabine; cortisone acetate; cytarabine; dacarbazine; dasatinib; daunorubicin; decitabine; desonide; dexamethasone; dichloroacetic acid; discodermolide; docetaxel; doxorubicin; epirubicin; epothilone; eribulin; estramustine; etoposide; exatecan; exisulind; ferruginol; floxuridine; fludarabine; fluocinonide; fluocortolone; fluorouracil; fosfestrol; fotemustine; gemcitabine; halometasone; halcinonide; hydrocortisone; hydrocortisone acetate; hydroxyurea; IT-101; idarubicin; ifosfamide; imiquimod; irinotecan; irofulven; ixabepilone; laniquidar; lapatinib; lenalidomide; lomustine; lurtotecan; mafosfamide; masoprocol; mechlorethamine;

melphalan; mercaptopurine; methylprednisone; mitomycin; mitotane; mitoxantrone; mometasone; nelarabine; nilotinib; oblimersen; oxaliplatin; PAC-I; methotrexate (RHEUMATREX®, Amethopterin); cyclophosphamide (CYTOXAN®) thalidomide (THALIDOMID®); paclitaxel; pemetrexed; pentostatin; pipobroman; pixantrone; plicamycin; prednisolone; prednisone; procarbazine; proteasome inhibitors (e.g.; bortezomib); raltitrexed; rebeccamycin; rubitecan; SN-38; salinosporamide A; satraplatin; streptozotocin; swainsonine; tariquidar; taxane; tegafur-uracil; temozolomide; testolactone; thioTEPA; tioguanine; topotecan; trabectedin; triamcinolone acetonide; tretinoin; triplatin tetranitrate; tris(2-chloroethyl)amme; troxacitabine; uracil mustard; valrubicin; vinblastine; vincristine; vinorelbine; vorinostat; zosuquidar; N-acetylcysteine; vitamin E; vitamin C; vitamin A; lutein; selenium glutathione; melatonin; a polyphenol; a carotenoid; coenzyme Q-IO; Ebselen (2-phenyl-1, 2-benzisoselenazol-3(2H)-one (also called PZ 51 or DR3305); L-methionine; azulenyl nitrones; L-(+)-Ergothioneine; CAPE (caffeic acid phenethyl ester); dimethylthiourea; dimethylsulfoxide; disufenton sodium; pentoxifylline; MCI-186; Ambroxol; U-83836E; MitoQ (mitoquinone mesylate); Idebenone (2-(10-hydroxydecyl)-5,6-dimethoxy-3-methyl-cyclohexa-2,5-diene-1,4-dione); desferrioxamine; hydroxybenzyl ethylene diamine; fullerenol-1, pyrrolidine dithiocarbamate; acetylcarnitine; lipoic acid; a stilbene; a chalcone; a flavone; an isoflavone; a flavanones; an anthocyanidin; a catechin; isonicotinamide; dipyridamole; ZM 336372; camptothecin; coumestrol; nordihydroguaiaretic acid; esculetin; SRT-1720; SRT-1460; SRT-2183; aminoguanidine; 1-Amino-2-hydroxyguanidine p-toluensulfate; GED; bromocriptine mesylate; dexamethasone; SDMA; ADMA; L-NMMA; L-NMEA; D-MMA; L-NIL; L-NNA; L-NPA; L-NAME; L-VNIO; diphenyleneiodonium chloride; 2-ethyl-2-thiopseudourea; haloperidol; L-NIO; MEG; SMT; SMTC; 7-Ni; nNOS inhibitor; 1,3-PBITU; L-thiocitrulline; TRIM; MTR-105; BBS-I; BBS-2; ONO-1714; GW273629; GW 274150; PPA250; AR-R17477; AR-R18512; spiroquinazolone; 1400W; S-NC; NTG; SNP; thapsigargin; VEGF; bradykinin; ATP; sphingosine-1-phosphate; estrogen; angiopoietin; acetylcholine; SIN-I; GEA 3162; GEA; GEA 5024; GEA 5538; SNAP; molsidomine; CNO-4; CNO-5; DEA/NO; IPA/NO; SPER/NO; SULFI/NO; OXI/NO; DETA/NO; nicorandil; minoxidil, levcromakalim; lemakalim; cromakalim; L-735,334; retigabine; flupirtine; BMS-204352; DMP-543; linopirdine; XE991; 4-AP; 3,4-DAP; E-4031; DIDS; Way 123,398; CGS-12066 A; dofetilide; sotalol; apamin; amiodarone; azimilide; bretylium; clofilium; tedisamil; ibutilide; sematilide; nifekalant; tamulustoxin; ATP; ADP; UTP; UDP; UDP-glucose; adenosine; 2-MESATP; 2-MESADP; ABMEATP; DATPAS; ATPrS; BZ-ATP; MRS2703; DENUFOSOL TETRASODIUM; MRS2365; MRS 2690; PSB 0474; A-317491; RO-3 (Roche); SURAMIN; PPADS; PPNDS; DIDS; pyridoxal-5-phosphate; 5-(3-bromophenyl)-1,3-dihydro-2H-benzofuro-[3,2-e]-1,4-diazepin-2-one; cibacron blue; basilen blue; ivermectin; A-438079; A-740003; NF023; NF449; NFI 1O; NF157; MRS 2179; NF279; MRS 2211; MRS 2279; MRS 2500 tetrasodium salt; TNP-ATP; tetramethylpyrazine; Ip51; jQγ-carboxymethylene ATP; βγ-chlorophosphomethylene ATP; KN-62; spinorphin; minocycline; SB-203580 (4-(4-Fluorophenyl)-2-(4-methylsulfmyl phenyl)-5-(4-pyridyl) 1H-imidazole); PD 169316 (4-(4-Fluorophenyl)-2-(4-nitrophenyl)-5-(4-pyridyl)-1H-imidazole); SB-202190 (4-(4-Fluorophenyl)-2-(4-hydroxyphenyl)-5-(4-pyridyl)1H-imidazole); RWJ 67657 (4-[4-(4-fluorophenyl)-1-(3-phenylpropyl)-5-(4-pyridinyl)-1H-imidazol -2-yl]-3-butyn-1-ol); SB-220025 (5-(2-Amino-4-pyrimidinyl)-4-(4-fluorophenyl)-1-(4-piperidinlyl)imidazole); D-JNKI-I ((D)-hJIPi75-i57-DPro-DPro-(D)-HIV-T AT57-48); AM-111 (Auris); SP600125 (anthra[1,9-cd]pyrazol-6(2H)-one); JNK Inhibitor I ((L)-HIV-TAT48-57-PP-JBD20); JNK Inhibitor III ((L)-HIV-TAT47-57-gaba-c-Junδ33-57); AS601245 (1,3-benzothiazol-2-yl (2-[[2-(3-pyridinyl) ethyl]amino]-4 pyrimidinyl) acetonitrile); JNK Inhibitor VI (H₂N-RPKRPTTLNLF-NH₂) (SEQ ID NO:711); JNK Inhibitor VIII (N-(4-Amino-5-cyano-6-ethoxypyridin-2-yl)-2-(2,5-dimethoxyphenyl)acetamide); JNK Inhibitor IX (N-(3-Cyano-4,5,6,7-tetrahydro-1-benzothien-2-yl)-1-naphthamide); dicumarol (3,3′-Methylenebis(4-hydroxycoumarin)); SC-236 (4-[5-(4-chlorophenyl)-3-(trifluoromethyl)-1H-pyrazol-1-yljbenzene-sulfonamide); CEP-1347 (Cephalon); CEP-11004 (Cephalon); an artificial protein comprising at least a portion of a Bcl-2 polypeptide; a recombinant FNK; V5 (also known as Bax inhibitor peptide V5); Bax channel blocker ((±)-1-(3,6-Dibromocarbazol-9-yl)-3-piperazin-1-yl-propan-2-ol); Bax inhibiting peptide P5 (also known as Bax inhibitor peptide P5); Kp7-6; FAIM(S) (Fas apoptosis inhibitory molecule-short); FAIM(L) (Fas apoptosis inhibitory molecule-long); Fas:Fc; FAP-I; NOK2; F2051; F1926; F2928; ZB4; Fas M3 mAb; EGF; 740 Y-P; SC 3036 (KKHTDDGYMPMSPGVA) (SEQ ID NO:712); PI3-kinase Activator (Santa Cruz Biotechnology, Inc.); Pam3Cys ((S)-(2,3-bis(palmitoyloxy)-(2RS)-propyl)-N-palmitoyl-(R)-Cys-(S)-Ser(S)-Lys4-OH, trihydrochloride); Actl (NF-KB activator 1); an anti-DcB antibody; Acetyl-11-keto-b-Boswellic Acid; Andrographolide; Caffeic Acid Phenethyl Ester (CAPE); Gliotoxin; Isohelenin; NEMO-Binding Domain Binding Peptide (DRQIKIWFQNRRMKWKKTALDWSWLQTE) (SEQ ID NO:713); NF-kB Activation Inhibitor (6-Amino-4-(4-phenoxyphenylethylamino)quinazoline); NF-kB Activation Inhibitor II (4-Methyl-N1-(3-phenylpropyl)benzene-1,2-diamine); NF-kB Activation Inhibitor III (3-Chloro-4-nitro-N-(5-nitro-2-thiazolyl)-benzamide); NF-kB Activation Inhibitor IV ((E)-2-Fluoro-4′-methoxystilbene); NF-kB Activation Inhibitor V (5-Hydroxy-(2,6-diisopropylphenyl)-1H-isoindole-1,3-dione); NF-kB SN50 (AAVALLPAVLLALL APVQRKRQKLMP) (SEQ ID NO:714); Oridonin; Parthenolide; PPM-18 (2-Benzoylamino-1,4-naphthoquinone); Rol06-9920; Sulfasalazine; TIRAP Inhibitor Peptide (RQIKIWFNRRMKWKKLQLRDAAPGGAIVS) (SEQ ID NO:715); Withaferin A; Wogonin; BAY 11-7082 ((E)3-[(4-Methylphenyl)sulfonyl]-2-propenenitrile); BAY 11-7085 ((E)3-[(4-t-Butylphenyl)sulfonyl]-2-propenenitrile); (E)-Capsaicin; Aurothiomalate (ATM or AuTM); Evodiamine; Hypoestoxide; IKK Inhibitor III (BMS-345541); IKK Inhibitor VII; IKK Inhibitor X; IKK Inhibitor II; IKK-2 Inhibitor IV;

IKK-2 Inhibitor V; IKK-2 Inhibitor VI; IKK-2 Inhibitor (SC-514); IkB Kinase Inhibitor Peptide; IKK-3 Inhibitor IX; ARRY-797 (Array BioPharma); SB-220025 (5-(2-Amino-4-pyrimidinyl)-4-(4-fluorophenyl)-1-(4-piperidinlyl)imidazole); SB-239063 (trans-4-[4-(4-Fluorophenyl)-5-(2-methoxy-4-pyrimidinyl)-1H-imidazol-1-yl]cyclohexanol); SB-202190 (4-(4-Fluorophenyl)-2-(4-hydroxyphenyl)-5-(4-pyridyl)1H-imidazole); JX-401 (-[2-Methoxy-4-(methylthio)benzoyl]-4-(phenylmethyl)piperidine); PD-169316 (4-(4-Fluorophenyl)-2-(4-nitrophenyl)-5-(4-pyridyl)-1H-imidazole); SKF-86002 (6-(4-Fluorophenyl)-2,3-dihydro-5-(4-pyridinyl)imidazo[2,1-b]thiazole dihydrochloride); SB-200646 (N-(I-Methyl-1H-indol-5-yl)-N′-3-pyridinylurea); CMPD-I (2′-Fluoro-N-(4-hydroxyphenyl)-[1,1′-biphenyl]-4-butanamide); EO-1428 ((2-Methylphenyl)-[4-[(2-amino-4-bromophenyl)amino]-2-chlorophenyl]methanone); SB-253080 (4-[5-(4-Fluorophenyl)-2-[4-(methylsulfonyl)phenyl]-1H-imidazol-4-yl]pyridine); SD-169 (1H-Indole-5-carboxamide); SB-203580 (4-(4-Fluorophenyl)-2-(4-methylsulfinyl phenyl)-5-(4-pyridyl) 1H-imidazole); TZP-101 (Tranzyme Pharma); TZP-102 (Tranzyme Pharma); GHRP-6 (growth hormone-releasing peptide-6); GHRP-2 (growth hormone-releasing peptide-2); EX-1314 (Elixir Pharmaceuticals); MK-677 (Merck); L-692,429 (Butanamide, 3-amino-3-methyl-N-(2,3,4,5-tetrahydro-2-oxo-1-((2′-(1H-tetrazol-5-yl)(1,1′-biphenyl)-4-yl)methyl)-1H-1-benzazepin-3-yl)-, (R)-); EP1572 (Aib-DTrp-DgTrp-CHO); diltiazem; metabolites of diltiazem; BRE (Brain and Reproductive organ-Expressed protein); verapamil; nimodipine; diltiazem; omega-conotoxin; GVIA; amlodipine; felodipine; lacidipine; mibefradil; NPPB (5-Nitro-2-(3-phenylpropylamino)benzoic Acid); flunarizine; erythropoietin; pipeline; hemin; brazilin; z-VAD-FMK (Benzyloxycarbonyl-Val-Ala-Asp(OMe)-fluoromethylketone); z-LEHD-FMK (benzyloxycarbonyl-Leu-Glu(OMe)-His-Asp(OMe)-fluoromethylketone); B-D-FMK (boc-aspartyl(Ome)-fluoromethylketone); Ac-LEHD-CHO (N-acetyl-Leu-Glu-His-Asp-CHO) (SEQ ID NO:716); Ac-IETD-CHO (N-acetyl-Ile-Glu-Thr-Asp-CHO) (SEQ ID NO:717); z-IETD-FMK (benzyloxycarbonyl-Ile-Glu(OMe)-Thr-Asp(OMe)-fluoromethyl ketone); FAM-LEHD-FMK (benzyloxycarbonyl Leu-Glu-His-Asp-fluoromethyl ketone); FAM-LETD-FMK (benzyloxycarbonyl Leu-Glu-Thr-Asp-fluoromethyl ketone); Q-VD-OPH (Quinoline-VaI-ASp-CH2-O-Ph); XIAP; cIAP-1; cIAP-2; ML-IAP; ILP-2; NAIP; Survivin; Bruce; IAPL-3; fortilin; leupeptine; PD-150606 (3-(4-Iodophenyl)-2-mercapto-(Z)-2-propenoic acid); MDL-28170 (Z-Val-Phe-CHO); calpeptin; acetyl-calpastatin; MG 132 (N-[(phenylmethoxy)carbonyl]-L-leucyI-N-[(IS)-1-formyl-3-methylbutyl]-L-leucinamide); MYODUR; BN 82270 (Ipsen); BN 2204 (Ipsen); AHLi-11 (Quark Pharmaceuticals), an mdm2 protein, pifithrin-α (1-(4-Methylphenyl)-2-(4,5,6,7-tetrahydro-2-imino-3(2H)-benzothiazolyl)ethanone); trans-stilbene; cis-stilbene; resveratrol; piceatannol; rhapontin; deoxyrhapontin; butein; chalcon; isoliquirtigen; butein; 4,2′,4′-trihydroxychalcone; 3,4,2′,4′,6′-pentahydroxychalcone; flavone; morin; fisetin; luteolin; quercetin; kaempferol; apigenin; gossypetin; myricetin; 6-hydroxyapigenin; 5-hydroxyflavone; 5,7,3′,4′,5′-pentahydroxyflavone; 3,7,3′,4′,5′-pentahydroxyflavone; 3,6,3′,4′-tetrahydroxyflavone; 7,3′,4′,5′-tetrahydroxyflavone; 3,6,2′,4′-tetrahydroxyflavone; 7,4′-dihydroxyflavone; 7,8,3′,4′-tetrahydroxy flavone; 3,6,2′,3′-tetrahydroxyflavone; 4′-hydroxyflavone; 5-hydroxyflavone; 5,4′-dihydroxyflavone; 5,7-dihydroxyflavone; daidzein; genistein; naringenin; flavanone; 3,5,7,3′,4′-pentahydroxyflavanone; pelargonidin chloride; cyanidin chloride; delphinidin chloride; (−)-epicatechin (Hydroxy Sites: 3,5,7,3′,4\hat{ }; (−)-catechin (Hydroxy Sites: 3,5,7,3′,40; (−)-gallocatechin (Hydroxy Sites: 3,5,7,3′,4′,5O(+)-catechin (Hydroxy Sites: 3,5,7,3′,4\hat{ }; (+)-epicatechin (Hydroxy Sites: 3,5,7,3′,41J; Hinokitiol (b-Thujaplicin; 2-hydroxy-4-isopropyl-2,4,6-cycloheptatrien-1-one); L-(+)-Ergothioneine ((S)-a-Carboxy-2,3-dihydro-N,N,N-trimethyl-2-thioxo-1H-imidazole4-ethanaminium inner salt); Caffeic Acid Phenyl Ester; MCI-186 (3-Methyl-1-phenyl-2-pyrazolin-5-one); HBED (N,N′-Di-(2-hydroxybenzy\hat{ }ethylenediamine-HN′-diacetic acid>>H2O); Ambroxol (trans-4-(2-Amino-3,5-dibromobenzylamino)cyclohexane-HCl; and U-83836E ((−)-2-((4-(2,6-di-1-Pyrrolidinyl-4-pyrimidinyl)-1-piperzainyl)methyl)-3,4-dihydro-2,5,7,8-tetramethyl-2H-1-benzopyran-6-ol*2HCl); /5-1-5-methyl-nicotinamide-2′-deoxyribose; /S-D-I ‘-5-methyl-nico-tinamide-2’-deoxyribofuranoside; /3-1′-4,5-dimethyl-nicotinamide-2′-de-oxyribose; /3-D-1′-4,5-dimethyl-nicotinamide-2′-deoxyribofuranoside; 1-Naphthyl PP 1(1-(1,1-Dimethyl ethyl)-3-(1-naphthalenyl)-1H-pyrazolo[3, 4-d]pyrimidin-4-amine); Lavendustin A (5-[[(2,5-Dihydroxyphenyl)methyl][(2-hydroxyphenyl)methyl]amino]-2-hydroxybenzoic acid); MNS (3 ,4-Methylenedioxy-b-nitrostyrene); PP 1 (1-(1,1-Dimethylethyl)-1-(4-methylphenyl)-1H-pyrazolo[3,4-d]pyrimidin-4-amine); PP2 (3-(4-chlorophenyl) 1-(1, 1-dimethylethyl)-1H-pyrazolo[3,4-d]pyrimidin-4-amine); KX1-004 (Kinex); KX1-005 (Kinex); KX1-136 (Kinex); KX1-174 (Kinex); KX1-141 (Kinex); KX2-328 (Kinex); KXI-306 (Kinex); KX1-329 (Kinex); KX2-391 (Kinex); KX2-377 (Kinex); ZD4190 (Astra Zeneca; N-(4-bromo-2-fluorophenyl)-6-methoxy-7-(2-(1H-1,2,3-triazol-1-yl)ethoxy)quinazolin-4-amine); AP22408 (Ariad Pharmaceuticals); AP23236 (Ariad Pharmaceuticals); AP23451 (Ariad Pharmaceuticals); AP23464 (Ariad Pharmaceuticals); AZD0530 (Astra Zeneca); AZM475271 (M475271; Astra Zeneca); Dasatinib (N-(2-chloro-6-methylphneyl)-2-(6-(4-(2-hydroxyethyl)-piperazin-1-yl)-2-methylpyrimidin-4-ylamino) thiazole-5-carboxamide); GN963 (trans-4-(6,7-dimethoxyquinoxalin-2ylamino)cyclohexanol sulfate); Bosutinib (4-((2,4-dichloro-5-methoxyphenyl)amino)-6-methoxy-7-(3-(4-methyl-1-piperazinyl)propoxy)-3-quinolinecarbonitrile); or combinations thereof.

### Radiolabels

In some embodiments, a cargo molecule comprises a radiolabel. As used herein, “radiolabel” refers to a moiety comprising a radioactive isotope of at least one element. In some embodiments, a radiolabel is one used in positron emission tomography (PET). In some embodiments, a radiolabel is one used in single-photon emission computed tomography (SPECT). In some embodiments, radioisotopes comprise ^{99m}Tc, ¹¹¹In, ⁶⁴Cu, ⁶⁷Ga, ¹⁸⁶Re, ¹⁸⁸Re, ¹⁵³Sm, ¹⁷⁷Lu, ⁶⁷Cu, ¹²³I, ¹²⁴I, ¹²⁵I, ¹¹C, ³N, ¹⁵O, ¹⁸F, ¹⁸⁶Re, ¹⁸⁸Re, ¹⁵³Sm, ¹⁶⁶Ho, ¹⁷⁷Lu, ¹⁴⁹Pm, ⁹⁰Y, ²¹³Bi, ¹⁰³Pd, ¹⁰⁹Pd, ¹⁵⁹Gd, 140La, ^{1 98}Au, ¹⁹⁹ Au, ¹⁶⁹Yb, ¹⁷⁵Yb, ¹⁶⁵Dy, ¹⁶⁶Dy, ⁶⁷Cu, ¹⁰⁵Rh, ¹¹¹Ag, ⁸⁹Zr, ²²⁵ Ac, or ¹⁹²Ir.

### Neurotropic Factors

In some embodiments, a cargo molecule comprises a factor having neurotrophic properties (e.g., neurotrophic proteins such as nerve growth factor (NGF), brain-derived neurotrophic factor (BDNF), neurotrophin-3 (NT-3), neurotrophin-4 (NT-4), glial cell line-derived neurotrophic factor (GDNF), ciliary neurotrophic factor (CNTF) as well as non-protein small molecules with neurotrophic properties).

### III. Linkers

In some embodiments, a cargo molecule (e.g., a fluorescent moiety, photosensitizing agent, or drug) is directly attached to the peptides disclosed herein, e.g. at the end of the targeting peptide. Alternatively, in some embodiments, a cargo molecule (e.g., a fluorescent moiety, photosensitizing agent, or drug) is indirectly attached to a peptide disclosed herein, e.g., via a linker.

In some embodiments of the nerve targeting peptide conjugates (with or without a linker) described herein, the peptide comprises or consists or the amino acid sequence set forth in any one of SEQ ID NOS: 1-66. In some embodiments, the peptide comprises or consists of the amino acid sequence set forth in any one of SEQ ID NOS:1-20. In some embodiments, the peptide comprises or consists of the amino acid sequence set forth in any one of SEQ ID NOS:21-40. In some embodiments, the peptide comprises or consists of the amino acid sequence set forth in any one of SEQ ID NOS:41-60. In some embodiments, the peptide comprises or consists of the amino acid sequence set forth in any one of SEQ ID NOS:61-66.

In some embodiments, the peptide comprises or consists or the amino acid sequence set forth in any one of SEQ ID NOS:1-710. In some embodiments, the peptide comprises or consists of the amino acid sequence set forth in any one of SEQ ID NOS:1-20 and 67-151. In some embodiments, the peptide comprises or consists of the amino acid sequence set forth in any one of SEQ ID NOS:21-40 and 152-326. In some embodiments, the peptide comprises or consists of the amino acid sequence set forth in any one of SEQ ID NOS:41-60 and 327-410. In some embodiments, the peptide comprises or consists of the amino acid sequence set forth in any one of SEQ ID NOS:61-66 and 411-435. In some embodiments, the peptide comprises or consists of the amino acid sequence set forth in any one of SEQ ID NOS:436-642. In some embodiments, the peptide comprises or consists of the amino acid sequence set forth in any one of SEQ ID NOS:643-710.

In some embodiments, the peptide comprises or consists or the amino acid sequence set forth in any one of SEQ ID NOS: 1, 2, 4-17, 23, 24, 29, 31, 34, 35, 37, 40, 41, 42, 44-47, 49, 50, 52-60, and 67-710.

In some embodiments, the peptide comprises or consists of the amino acid sequence set forth in any one of SEQ ID NOS: 1, 2, 4-17, and 67-151.

In some embodiments, the peptide comprises or consists of the amino acid sequence set forth in any one of SEQ ID NOS:23, 24, 29, 31, 34, 35, 37, 40, and 152-326.

In some embodiments, the peptide comprises or consists of the amino acid sequence set forth in any one of SEQ ID NOS: 41, 42, 44-47, 49, 50, 52-60, and 327-410.

In some embodiments, the peptide comprises or consists of the amino acid sequence set forth in any one of SEQ ID NOS:411-435.

As used herein, a “linker” is any molecule capable of binding (e.g., covalently) to a targeting molecule disclosed herein. Linkers include, but are not limited to, straight or branched-chain carbon linkers, heterocyclic carbon linkers, amino acid linkers (e.g., D- or L-amino acid), lipophilic residues, peptide linkers, peptide nucleic acid linkers, hydrazone linkers, SPDB disulfide, sulfo-SPDB, maleimidomethyl cyclohexane-1-carboxylate (MCC), aminohexanoic acid linkers, and polyether linkers (e.g., PEG). For example, poly(ethylene glycol) linkers are available from Quanta Biodesign, Powell, OH. These linkers optionally have amide linkages, sulfhydryl linkages, or hetero functional linkages.

In some embodiments, the linker binds to a targeting molecule disclosed herein by a covalent linkage. A linker may connect a cargo molecule to the peptide by forming a covalent linkage to the cargo molecule at one location and a covalent linkage to the peptide at another location. The covalent linkages can be formed by reaction between functional groups on the linker and functional groups on the peptide and on the cargo molecule. In some embodiments, the covalent linkage comprises an ether bond, thioether bond, amine bond, amide bond, carbon-carbon bond, carbon-nitrogen bond, carbon-oxygen bond, or carbon-sulfur bond.

In some embodiments, the linker is flexible. In some embodiments, the linker is rigid. In some embodiments, the linker has segment(s) of flexibility and segment(s) of rigidity.

In some embodiments, the linker comprises a linear structure. In some embodiments, the linker comprises a non-linear structure. In some embodiments, the linker comprises a branched structure. In some embodiments, the linker comprises a cyclic structure.

In some embodiments, the linker is an alkyl. In some embodiments, the linker is heteroalkyl.

In some embodiments, the linker is an alkylene. In some embodiments, the linker is an alkenylene. In some embodiments, the linker is an alkynylene. In some embodiments, the linker is a heteroalkylene.

An “alkyl” group refers to an aliphatic hydrocarbon group. The alkyl moiety may be a saturated alkyl or an unsaturated alkyl. Depending on the structure, an alkyl group can be a monoradical or a diradical (i.e., an alkylene group).

The “alkyl” moiety may have 1 to 10 carbon atoms (whenever it appears herein, a numerical range such as “1 to 10” refers to each integer in the given range; e.g., “1 to 10 carbon atoms” means that the alkyl group may consist of 1 carbon atom, 2 carbon atoms, 3 carbon atoms, etc., up to and including 10 carbon atoms, although the present definition also covers the occurrence of the term “alkyl” where no numerical range is designated). The alkyl group could also be a “lower alkyl” having 1 to 6 carbon atoms. The alkyl group of the compounds described herein may be designated as “Ci-C4 alkyl” or similar designations. By way of example only, “C1-C4 alkyl” indicates that there are one to four carbon atoms in the alkyl chain, i.e., the alkyl chain is selected from methyl, ethyl, propyl, iso-propyl, n-butyl, iso-butyl, sec -butyl, and t-butyl. Typical alkyl groups include, but are in no way limited to, methyl, ethyl, propyl, isopropyl, butyl, isobutyl, tertiary butyl, pentyl, hexyl, ethenyl, propenyl, butenyl, and the like.

In some embodiments, the linker comprises a ring structure (e.g., an aryl). As used herein, the term “ring” refers to any covalently closed structure. Rings include, for example, carbocycles (e.g., aryls and cycloalkyls), heterocycles (e.g., heteroaryls and non-aromatic heterocycles), aromatics (e.g. aryls and heteroaryls), and non-aromatics (e.g., cycloalkyls and non-aromatic heterocycles). Rings can be optionally substituted. Rings can be monocyclic or polycyclic.

As used herein, the term “aryl” refers to an aromatic ring wherein each of the atoms forming the ring is a carbon atom. Aryl rings can be formed by five, six, seven, eight, nine, or more than nine carbon atoms. Aryl groups can be optionally substituted. Examples of aryl groups include, but are not limited to phenyl, naphthalenyl, phenanthrenyl, anthracenyl, fluorenyl, and indenyl. Depending on the structure, an aryl group can be a monoradical or a diradical (i.e., an arylene group).

The term “cycloalkyl” refers to a monocyclic or polycyclic non-aromatic radical, wherein each of the atoms forming the ring (i.e. skeletal atoms) is a carbon atom. Cycloalkyls may be saturated, or partially unsaturated. Cycloalkyl groups include groups having from 3 to 10 ring atoms. Cycloalkyls include, but are not limited to, cyclopropyl, cyclobutyl, cyclopentyl, cyclohexyl, cycloheptyl, and cyclooctyl.

In some embodiments, the ring is a cycloalkane. In some embodiments, the ring is a cycloalkene.

In some embodiments, the ring is an aromatic ring. The term “aromatic” refers to a planar ring having a delocalized x-electron system containing 4n+2π electrons, where n is an integer. Aromatic rings can be formed from five, six, seven, eight, nine, or more than nine atoms. Aromatics can be optionally substituted. The term “aromatic” includes both carbocyclic aryl (e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups.

In some embodiments, the ring is a heterocycle. The term “heterocycle” refers to heteroaromatic and heteroalicyclic groups containing one to four heteroatoms each selected from O, S and N, wherein each heterocyclic group has from 4 to 10 atoms in its ring system, and with the proviso that the ring of said group does not contain two adjacent O or S atoms. Non-aromatic heterocyclic groups include groups having only 3 atoms in their ring system, but aromatic heterocyclic groups must have at least 5 atoms in their ring system. The heterocyclic groups include benzo-fused ring systems. An example of a 3-membered heterocyclic group is aziridinyl. An example of a 4-membered heterocyclic group is azetidinyl (derived from azetidine). An example of a 5-membered heterocyclic group is thiazolyl. An example of a 6-membered heterocyclic group is pyridyl, and an example of a 10-membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and faropyridinyl. The foregoing groups, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl or imidazol-3-yl (both N-attached) or imidazol-2-yl, imidazol-4-yl or imidazol-5-yl (all C-attached). The heterocyclic groups include benzo-fused ring systems and ring systems substituted with one or two oxo (=0) moieties such as pyrrolidin-2-one. Depending on the structure, a heterocycle group can be a monoradical or a diradical (i.e., a heterocyclene group).

In some embodiments, the ring is fused. The term “fused” refers to structures in which two or more rings share one or more bonds, hi some embodiments, the ring is a dimer. In some embodiments, the ring is a trimer. In some embodiments, the ring is a substituted.

The term “carbocyclic” or “carbocycle” refers to a ring wherein each of the atoms forming the ring is a carbon atom. Carbocycle includes aryl and cycloalkyl. The term thus distinguishes carbocycle from heterocycle (“heterocyclic”) in which the ring backbone contains at least one atom which is different from carbon (i.e., a heteroatom).

Heterocycle includes heteroaryl and heterocycloalkyl. Carbocycles and heterocycles can be optionally substituted.

In some embodiments, the linker is substituted. The term “optionally substituted” or “substituted” means that the referenced group may be substituted with one or more additional group(s) individually and independently selected from C1-C6alkyl, C3-C6cycloalkyl, aryl, heteroaryl, C2-C6heteroalicyclic, hydroxy, C1-C6alkoxy, aryloxy, C1-C6alkylthio, arylthio, C1-C6alkylsulfoxide, arylsulfoxide, C1-C6alkylsulfone, arylsulfone, cyano, halo, C2-C8acyl, C2-C8acyloxy, nitro, C1-C6haloalkyl, C1-C6fluoroalkyl, and amino, including C1-C6alkylamino, and the protected derivatives thereof. By way of example, an optional substituents may be LSRS, wherein each Ls is independently selected from a bond, —O—, —C(═O)—, —S—, —S(═O)—, —S(O)2—, —NH—, —NHC(O)—, —C(O)NH—, S(O)2NH—, —NHS(O)2—, —OC(O)NH—, —NHC(O)O—, —(CpC6alkyl)-, or -(C2-C6alkenyl)-; and each Rs is independently selected from H, (C1-C4alkyl), (C3-C8cycloalkyl), heteroaryl, aryl, and C1-C6heteroalkyl. Optionally substituted non-aromatic groups may be substituted with one or more oxo (=0). The protecting groups that may form the protective derivatives of the above substituents are known to those of skill in the art.

In some embodiments, a bifunctional linker having one functional group reactive with a group on one molecule (e.g., a targeting molecule), and another group reactive on the other molecule (e.g., a fluorescent moiety or a drug), is used to form the desired conjugate. Alternatively, in some embodiments, derivatization is performed to provide functional groups. Thus, for example, procedures for the generation of free sulfhydryl groups on peptides are also known (See U.S. Pat. No. 4,659,839). A linker may alternatively comprise a heterobifunctional crosslinker comprising two or more different reactive groups that form a heterocyclic ring that can interact with a targeting molecule. For example, a heterobifunctional crosslinker such as cysteine may comprise an amine reactive group and a thiol-reactive group can interact with an aldehyde on a derivatized targeting molecule. Additional combinations of reactive groups suitable for heterobifunctional crosslinkers include, for example, amine- and sulfhydryl reactive groups; carbonyl and sulfhydryl reactive groups; amine and photoreactive groups; sulfhydryl and photoreactive groups; carbonyl and photoreactive groups; carboxylate and photoreactive groups; and arginine and photoreactive groups. Examples of a heterobifunctional crosslinker include N-Succinimidyl 4-(2-pyridyldithio)butanoate (SPDB) and maleimidomethyl cyclohexane-1-carboxylate (MCC).

In some embodiments, a linker is cleavable. In some embodiments, the linker is non-cleavable. A linker can be chemically stable to extracellular environments, for example, chemically stable in the blood stream, and/or may include linkages that are not stable. A linker can include linkages that are designed to cleave and/or immolate or otherwise breakdown specifically or non-specifically inside cells. A cleavable linker can be sensitive to enzymes at a specific site, such as by extracellular proteases.

A cleavable linker can include a valine-citrulline peptide, a valine-alanine peptide, a phenylalanine-lysine or other peptide, such as a peptide that forms a protease recognition and cleavage site. Such a peptide-containing linker can contain a pentafluorophenyl group. A peptide-containing linker can include a succimide or a maleimide group. A peptide-containing linker can include a para aminobenzoic acid (PABA) group. A peptide-containing linker can include an aminobenzyloxycarbonyl (PABC) group. A peptide-containing linker can include a PABA or PABC group and a pentafluorophenyl group. A peptide-containing linker can include a PABA or PABC group and a succinimide group. A peptide-containing linker can include a PABA or PABC group and a maleimide group.

A non-cleavable linker is generally protease-insensitive and insensitive to intracellular processes. A non-cleavable linker can include a maleimide group. A non-cleavable linker can include a succinimide group. A non-cleavable linker can be maleimido-alkyl-C(O)-linker. A non-cleavable linker can be maleimidocaproyl linker. A maleimidocaproyl linker can be N-maleimidomethylcyclohexane-1-carboxylate. A maleimidocaproyl linker can include a succinimide group. A maleimidocaproyl linker can include pentafluorophenyl group.

In some embodiments, a peptide linker consisting of one or more amino acids is used to join the targeting molecule and a fluorescent moiety or drug. Generally the peptide linker will have no specific biological activity other than to join the molecules or to preserve some minimum distance or other spatial relationship between them. However, the constituent amino acids of the linker may be selected to influence some property of the molecule such as the folding, net charge, or hydrophobicity. In some embodiments the peptide linker is relatively short, typically less than about 10 amino acids, preferably less than about 8 amino acids and more preferably less than 5 amino acids. Non-limiting illustrative examples include glycine and glycine-serine linkers which can be added to the C-terminus of a peptide. In some embodiments, a peptide linker comprises a cysteine residue or an unnatural amino acid residue (e.g., selenocysteine (Sec), p-acetophenylalanine (pAcF), p-azidomethyl-L-phenylalanine (pAMF), and azido-lysine (AzK)) for site specific conjugation. In some embodiments, a peptide linker is a glycine-glycine-glycine-cysteine (GGGC) (SEQ ID NO:718) linker, a glycine-glycine-cysteine (GGC) linker, a glycine-glycine (GG) linker, or a cysteine (C) linker. In some embodiments, the GGGC (SEQ ID NO:718), GGC, GG, or C linker is added to the N-terminus or C-terminus of a peptide.

### IV. Further Modifications

In some embodiments, the peptides of the present disclosure are optionally conjugated to high molecular weight molecules that increase the multivalency and avidity of labeling. In some embodiments, the high molecular weight molecules are water-soluble polymers. Examples of suitable water-soluble polymers include, but are not limited to, peptides, saccharides, poly(vinyls), poly(ethers), poly(amines), poly(carboxylic acids) and the like. In some embodiments, the water-soluble polymers is dextran, polyethylene glycol (PEG), polyoxyalkylene, polysialic acid, starch, or hydroxyethyl starch. Any suitable method is used to conjugate peptides to water-soluble polymers (see, Hermanson G., Bioconjugate Techniques 2nd Ed., Academic Press, Inc. 2008). In some embodiments, the peptide-high molecular weight molecule conjugate further comprises a cargo molecule.

In some embodiments, one or more additional amino acid residues can be added to the N-terminus, the C-terminus, or both N-terminus and C-terminus of the peptides disclosed herein, thus producing a longer peptide. In some embodiments, 1, 2, 3, 4, 5, 6, 7, 8, 9, 10, 11, 12, 13, 14, 15 or more amino acid residues are added to the peptides disclosed herein. In some embodiments, about 1-15, 1-10, 1-5, or 1-3 amino acids are added to the peptides disclosed herein. In some embodiments, additional amino acids that are added to the peptides of the present disclosure may act as a spacer to provide a variable distance between the peptide and the cargo molecule.

In some embodiments, the nerve targeting peptide conjugates of the present invention are modified to increase solubility. Peptide modifications that increase solubility include addition of hydrophilic amino acid(s), a PEG moiety, or both. In some embodiments, a PEG moiety is 8-Amino-3,6-dioxaoctanoic acid (AEEA); 12-amino-4,7,10-trioxadodecanoic acid; or 15-amino-4,7,10,13-tetraoxapenta-decanoic acid. In some embodiments, about one to ten (e.g., one, two, three, four, five, six, seven, eight, nine, or ten) hydrophilic amino acids may be added to the N-terminus, C-terminus, an internal position, or any combination thereof, of the peptide component of the nerve targeting peptide conjugate to increase solubility. Hydrophilic amino acids include D, E, H, K, N, Q, R, S, T, and G. In some embodiments, the peptide comprises a K, KK, KKK, KKKK (SEQ ID NO:719), G, GG, GGG, or GGGG (SEQ ID NO:720) at the N-terminus or C-terminus.

### V. Pharmaceutical Compositions

Disclosed herein, in certain embodiments, are pharmaceutical compositions comprising a nerve targeting peptide conjugate disclosed herein. Pharmaceutical compositions herein are formulated using one or more physiologically acceptable carriers including excipients and auxiliaries which facilitate processing of the active agents into preparations which are used pharmaceutically. Proper formulation is dependent upon the route of administration chosen. A summary of pharmaceutical compositions is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N. Y., 1980; and Pharmaceutical Dosage Forms and Drug Delivery Systems, Seventh Ed. (Lippincott Williams & Wilkins, 1999).

In some embodiments of pharmaceutical compositions comprising the nerve targeting peptide conjugates described herein, the peptide comprises or consists or the amino acid sequence set forth in any one of SEQ ID NOS:1-66. In some embodiments of pharmaceutical compositions comprising the nerve targeting peptide conjugates described herein, the peptide comprises or consists of the amino acid sequence set forth in any one of SEQ ID NOS: 1-20. In some embodiments of pharmaceutical compositions comprising the nerve targeting peptide conjugates described herein, the peptide comprises or consists of the amino acid sequence set forth in any one of SEQ ID NOS:21-40. In some embodiments of pharmaceutical compositions comprising the nerve targeting peptide conjugates described herein, the peptide comprises or consists of the amino acid sequence set forth in any one of SEQ ID NOS:41-60. In some embodiments of pharmaceutical compositions comprising the nerve targeting peptide conjugates described herein, the peptide comprises or consists of the amino acid sequence set forth in any one of SEQ ID NOS:61-66.

In some embodiments of pharmaceutical compositions comprising the nerve targeting peptide conjugates described herein, the peptide comprises or consists or the amino acid sequence set forth in any one of SEQ ID NOS: 1-710. In some embodiments of pharmaceutical compositions comprising the nerve targeting peptide conjugates described herein, the peptide comprises or consists of the amino acid sequence set forth in any one of SEQ ID NOS: 1-20 and 67-151. In some embodiments of pharmaceutical compositions comprising the nerve targeting peptide conjugates described herein, the peptide comprises or consists of the amino acid sequence set forth in any one of SEQ ID NOS:21-40 and 152-326. In some embodiments of pharmaceutical compositions comprising the nerve targeting peptide conjugates described herein, the peptide comprises or consists of the amino acid sequence set forth in any one of SEQ ID NOS:41-60 and 327-410. In some embodiments of pharmaceutical compositions comprising the nerve targeting peptide conjugates described herein, the peptide comprises or consists of the amino acid sequence set forth in any one of SEQ ID NOS:61-66 and 411-435. In some embodiments of pharmaceutical compositions comprising the nerve targeting peptide conjugates described herein, the peptide comprises or consists of the amino acid sequence set forth in any one of SEQ ID NOS:436-642. In some embodiments of pharmaceutical compositions comprising the nerve targeting peptide conjugates described herein, the peptide comprises or consists of the amino acid sequence set forth in any one of SEQ ID NOS:643-710.

In some embodiments of pharmaceutical compositions comprising the nerve targeting peptide conjugates described herein, the peptide comprises or consists or the amino acid sequence set forth in any one of SEQ ID NOS: 1, 2, 4-17, 23, 24, 29, 31, 34, 35, 37, 40, 41, 42, 44-47, 49, 50, 52-60, and 67-710.

In some embodiments of pharmaceutical compositions comprising the nerve targeting peptide conjugates described herein, the peptide comprises or consists of the amino acid sequence set forth in any one of SEQ ID NOS: 1, 2, 4-17, and 67-151.

In some embodiments of pharmaceutical compositions comprising the nerve targeting peptide conjugates described herein, the peptide comprises or consists of the amino acid sequence set forth in any one of SEQ ID NOS:23, 24, 29, 31, 34, 35, 37, 40, and 152-326.

In some embodiments of pharmaceutical compositions comprising the nerve targeting peptide conjugates described herein, the peptide comprises or consists of the amino acid sequence set forth in any one of SEQ ID NOS: 41, 42, 44-47, 49, 50, 52-60, and 327-410.

In some embodiments of pharmaceutical compositions comprising the nerve targeting peptide conjugates described herein, the peptide comprises or consists of the amino acid sequence set forth in any one of SEQ ID NOS: 411-435.

In certain embodiments, a pharmaceutical composition disclosed herein further comprises a pharmaceutically acceptable diluent(s), excipient(s), or carrier(s). In some embodiments, the pharmaceutical compositions include other medicinal or pharmaceutical agents, carriers, adjuvants, such as preserving, stabilizing, wetting or emulsifying agents, solution promoters, salts for regulating the osmotic pressure, and/or buffers. In addition, the pharmaceutical compositions also contain other therapeutically valuable substances.

In certain embodiments, nerve targeting peptide conjugates disclosed herein are delivered to a subject via a drug delivery vehicle or carrier. In some embodiments, a delivery vehicle is made from natural or synthetic materials or both. In some embodiments, a delivery vehicle is a nanoparticle, microparticle, polymeric micelle, nanocapsule, dendrimer, large PEG, nanogel, liposome, fullerene, nanostructured lipid carrier, nanoshell, quantum dot, protein-based nanocarriers (e.g., albumin, elastin, gliadin, legumin, zein, soy protein, milk protein, whey based nanocarriers), organic nanocarrier (e.g., gelatin, dextran, guar gum, chitosan, collagen), polysaccharide based carrier (e.g., dextran, chitosan, pectin), lipid emulsion, or a combination thereof.

In certain embodiments, a pharmaceutical composition disclosed herein is administered to a subject by any suitable administration route, including but not limited to, topical, oral, intrarectal, intravaginal, intranasal, inhalation, parenteral (intravenous, intraarterial, subcutaneous, intraperitoneal, intramuscular, intravascular, intrathecal, intravitreal, infusion) administration. In certain embodiments, a pharmaceutical composition disclosed herein is administered to a subject is administered locally or systemically. In embodiments, a pharmaceutical composition disclosed herein is administered intravenously.

Formulations suitable for intramuscular, subcutaneous, or intravenous injection include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propylene glycol, polyethylene-glycol, glycerol, cremophor and the like), suitable mixtures thereof, vegetable oils (such as olive oil) and injectable organic esters such as ethyl oleate. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. Bioavailability enhancers may include penetration or permeation enhancers. See, e.g., Muheem et al., 2016. Saudi Pharm. J. 24, 413-428; Brayden et al 2020, Adv Drug Deliv Rev 2020 May 29.S0169-409X(20)30040-5. doi. 10.1016/j.addr.2020.05.007. Epub ahead of print. Ibrahim et al. 2020, J. Pham. Sci. 28, 403-416 In some formulations, proper fluidity can be maintained, for example, by the use of a coating such as lecithin, by the maintenance of the required particle size in the case of dispersions, and by the use of surfactants. Formulations suitable for subcutaneous injection also contain optional additives such as preserving, wetting, emulsifying, and dispensing agents, as are known in the art. In embodiments, formulations suitable for intravenous injection may be prepared in aqueous solutions, such as saline buffers and other physiologically compatible buffers known in the art.

Parenteral injections optionally involve bolus injection or continuous infusion. Formulations for injection are optionally presented in unit dosage form, e.g., in ampoules or in multi dose containers, with an added preservative. In some embodiments, the pharmaceutical composition described herein are in a form suitable for parenteral injection as sterile suspensions, solutions or emulsions in oily or aqueous vehicles, and contain formulatory agents such as suspending, stabilizing and/or dispersing agents. Pharmaceutical formulations for parenteral administration include aqueous solutions of an active agent in water soluble form. Additionally, suspensions are optionally prepared as appropriate oily injection suspensions.

In embodiments, a pharmaceutical composition herein is administered orally. Dosage forms suitable for oral administration can be solid or liquid and may include for example, a pill, capsule, troche, tablet, caplet, gel caplet (gelcap), syrup, an aqueous suspension or solution, a chewable form, a swallowable form, a dissolvable form, an effervescent, a granulated form, and an oral liquid solution. In a specific embodiment, the dosage form is a solid dosage form, and more specifically, comprises a tablet or capsule, or is orally administered by a vehicle or carrier as disclosed herein.

In some embodiments, the pharmaceutical composition described herein is in unit dosage forms suitable for single administration of precise dosages. In unit dosage form, the formulation is divided into unit doses containing appropriate quantities of an active agent disclosed herein. In some embodiments, the unit dosage is in the form of a package containing discrete quantities of the formulation. Non-limiting examples are packaged tablets or capsules, and powders in vials or ampoules. In some embodiments, aqueous suspension compositions are packaged in single-dose non-reclosable containers. Alternatively, multiple-dose reclosable containers are used, in which case it is typical to include a preservative in the composition. By way of example only, formulations for parenteral injection are presented in unit dosage form, which include, but are not limited to ampoules, or in multi dose containers, with an added preservative. In some embodiments, the nerve targeting peptide conjugate is administered via systemic intravenous injection into human patients.

### VI. Methods of Use

In another aspect, the present disclosure provides methods of delivering a cargo molecule to a neuron or nerve, comprising contacting the neuron or nerve with the nerve targeting peptide conjugates or pharmaceutical compositions described herein. Such methods may be used to label or identify a neuron or nerve, delivery a drug to a neuron or nerve, or delivery a photosensitizing agent to a neuron or nerve.

In some embodiments, the contacting occurs in vivo. In some embodiments, the contacting occurs in vitro.

In some embodiments of the methods described herein, the nerve targeting peptide conjugates are administered to a subject, as described herein, including humans and mammals (e.g., mice, rats, pigs, cats, dogs, and horses). In some embodiments, subjects are mammals. In some embodiments, subjects are primates. In some embodiments, subjects are humans. In some embodiments, human subjects are pediatric subjects (age 21 years and younger), adult subjects (age 22 years to 65 years), or geriatric subjects (age 65 years and above).

In some embodiments of the methods described herein, the peptide comprises or consists or the amino acid sequence set forth in any one of SEQ ID NOS: 1-66. In some embodiments of the methods described herein, the peptide comprises or consists of the amino acid sequence set forth in any one of SEQ ID NOS: 1-20. In some embodiments of the methods described herein, the peptide comprises or consists of the amino acid sequence set forth in any one of SEQ ID NOS:21-40. In some embodiments of the methods described herein, the peptide comprises or consists of the amino acid sequence set forth in any one of SEQ ID NOS:41-60. In some embodiments of the methods described herein, the peptide comprises or consists of the amino acid sequence set forth in any one of SEQ ID NOS:61-66.

In some embodiments of the methods described herein, the peptide comprises or consists or the amino acid sequence set forth in any one of SEQ ID NOS: 1-710. In some embodiments of the methods described herein, the peptide comprises or consists of the amino acid sequence set forth in any one of SEQ ID NOS: 1-20 and 67-151. In some embodiments of the methods described herein, the peptide comprises or consists of the amino acid sequence set forth in any one of SEQ ID NOS:21-40 and 152-326. In some embodiments of the methods described herein, the peptide comprises or consists of the amino acid sequence set forth in any one of SEQ ID NOS:41-60 and 327-410. In some embodiments of the methods described herein, the peptide comprises or consists of the amino acid sequence set forth in any one of SEQ ID NOS:61-66 and 411-435. In some embodiments of the methods described herein, the peptide comprises or consists of the amino acid sequence set forth in any one of SEQ ID NOS:436-642. In some embodiments of the methods described herein, the peptide comprises or consists of the amino acid sequence set forth in any one of SEQ ID NOS:643-710.

In some embodiments of the methods described herein, the peptide comprises or consists or the amino acid sequence set forth in any one of SEQ ID NOS: 1, 2, 4-17, 23, 24, 29, 31, 34, 35, 37, 40, 41, 42, 44-47, 49, 50, 52-60, and 67-710.

In some embodiments of the methods described herein, the peptide comprises or consists of the amino acid sequence set forth in any one of SEQ ID NOS: 1, 2, 4-17 and 67-151.

In some embodiments of the methods described herein, the peptide comprises or consists of the amino acid sequence set forth in any one of SEQ ID NOS:23, 24, 29, 31, 34, 35, 37, 40, and 152-326.

In some embodiments of the methods described herein, the peptide comprises or consists of the amino acid sequence set forth in any one of SEQ ID NOS: 41, 42, 44-47, 49, 50, 52-60, and 327-410.

In some embodiments of the methods described herein, the peptide comprises or consists of the amino acid sequence set forth in any one of SEQ ID NOS: 411-435.

In some embodiments, the present disclosure provides methods of labeling or identifying a neuron or nerve (or component of either) by contacting a neuron or nerve with nerve targeting peptide conjugate described herein.

In some embodiments, the cargo molecule of the nerve targeting peptide conjugate comprises a fluorescent moiety described herein. In some embodiments, the fluorescent moiety comprises a fluorescent protein, a fluorescent peptide, a fluorophore, or any combination thereof.

In some embodiments, a nerve targeting peptide conjugate is administered in combination (simultaneously, concurrently, or serially) with a fluorescent moiety (e.g., fluorescent moiety that is not conjugated to the nerve target peptide conjugate, or “free” fluorescent moiety). In some embodiments, the fluorescent moiety is a fluorescein, e.g., carboxyfluorescein.

In some embodiments of the methods of labeling or identifying a neuron or nerve described herein, the neuron or nerve is labeled for identification during surgery on the subject. In some embodiments, the surgery is related to a traumatic injury in the subject. In some embodiments, the surgery is related to an infection in the subject. In some embodiments, the surgery is plastic surgery e.g., cosmetic or reconstructive surgery, in the subject.

In some embodiments, the surgery is head, neck, skull base, spinal, prostate, heart, kidney, hand, arm, foot, leg, lower abdominal, or gynecological surgery.

In some embodiments the surgery is cancer surgery. In some embodiments the cancer is selected from the group consisting of prostate cancer, liver cancer (HCC), colorectal cancer, ovarian cancer, endometrial cancer, breast cancer, pancreatic cancer, stomach cancer, cervical cancer, head and neck cancer, thyroid cancer, testis cancer, urothelial cancer, lung cancer, melanoma, testicular germ cell tumors, mesothelioma, and esophageal cancer. In some embodiments, the cancer is prostate cancer.

In some embodiments, the method comprises administering a nerve targeting peptide conjugate disclosed herein to a subject that will undergo surgery. In some embodiments, the method comprises administering a nerve targeting peptide conjugate disclosed herein to a subject that is currently undergoing surgery. In some embodiments, a nerve targeting peptide conjugate disclosed herein is administered to a patient systemically, for example, by intravenous injection. In some embodiments, a nerve targeting peptide conjugate disclosed herein is administered to a patient locally. In some embodiments, a nerve targeting peptide conjugate disclosed herein is administered to a patient orally.

Disclosed herein, in certain embodiments, are methods of targeted drug delivery. In some embodiments, a nerve targeting peptide conjugate disclosed herein delivers a drug to a specific target. In some embodiments, a nerve targeting peptide conjugate disclosed herein delivers a drug to a neuron or nerve.

In some embodiments, the drug is an agent that reduces pain (either the perception of pain or activity of a painful stimulant). In some embodiments, the drug is an anesthetic. In some embodiments, the drug is benzocaine; carticaine; cinchocaine; cyclomethycaine; lidocaine; prilocaine; propxycaine; proparacaine; tetracaine; tocainide; and trimecaine; or a combination thereof.

In some embodiments, the drug is an agent that modulates death (e.g., via apoptosis or necrosis) of a neuron or nerve. In some embodiments, the drug is a cytotoxic agent. In some embodiments, the drug is methotrexate (RHEUMATREX®, Amethopterin); cyclophosphamide (CYTOXAN®);thalidomide (THALIDOMID®); paclitaxel; pemetrexed; pentostatin; pipobroman; pixantrone; plicamycin; procarbazine; proteasome inhibitors (e.g.; bortezomib); raltitrexed; rebeccamycin; rubitecan; SN-38; salinosporamide A; satraplatin; streptozotocin; swainsonine; tariquidar; taxane; tegafur-uracil; temozolomide; testolactone; tbioTEPA; tioguanine; topotecan; trabectedin; tretinoin; triplatin tetranitrate; tris(2-chloroethyl)amine; troxacitabine; uracil mustard; valrubicin; vinblastine; vincristine; vinorelbine; vorinostat; zosuquidar; or a combination thereof. In some embodiments, the drug is a pro-apoptotic agent. In some embodiments, the drug is an anti-apoptotic agent. In some embodiments, the drug is selected from minocycline; SB-203580 (4-(4-Fluorophenyl)-2-(4-methylsulfmyl phenyl)-5-(4-pyridyl)1H-imidazole); PD 169316 (4-(4-Fluorophenyl)-2-(4-nitrophenyl)-5-(4-pyridyl)-1H-imidazole); SB 202190 (4-(4-Fluorophenyl)-2-(4-hydroxyphenyl)-5-(4-pyridyl)1H-imidazole); RWJ 67657 (4-[4-(4-fluorophenyl)-1-(3-phenylpropyl)-5-(4-pyridinyl)-1H-imidazol -2-yl]-3-butyn-l-ol); SB 220025 (5-(2-Amino-4-pyrimidinyl)-4-(4-fluorophenyl)-1-(4-piperidinlyl)imidazole); D-JNKI-1((D)-hJIP 175_i 57-DPrO-DPrO-(D)-HIV-TAT57-48); AM-111 (Auris); SP600125 (anthra[1,9-cd]pyrazol-6(2H)-one); JNK Inhibitor I ((L)-HIV-T AT48-57-PP-JBD20); JNK Inhibitor III ((L)-HIV-TAT47-57-gaba-c-Junδ33-57); AS601245 (1,3-benzothiazol-2-yl (2-[[2-(3-pyridinyl) ethyl]amino]-4 pyrimidinyl) acetonitrile); JNK Inhibitor VI (H2N-RPKRPTTLNLF-NH2) (SEQ ID NO:711); JNK Inhibitor VIII (N-(4-Amino-5-cyano-6-ethoxypyridin-2-yl)-2-(2,5-dimethoxyphenyl)acetamide); JNK Inhibitor IX (N-(3-Cyano-4,5,6,7-tetrahydro-1-benzothien-2-yl)-1-naphthamide); dicumarol (3,3′-Methylenebis(4-hydroxycoumarin)); SC-236 (4-[5-(4-chlorophenyl)-3-(trifluoromethyl)-1H-pyrazol-1-yl]benzene-sulfonamide); CEP-1347 (Cephalon); CEP-11004 (Cephalon); an artificial protein comprising at least a portion of a Bcl-2 polypeptide; a recombinant FNK; V5 (also known as Bax inhibitor peptide V5); Bax channel blocker ((±)-1-(3,6-Dibromocarbazol-9-yl)-3-piperazin-1-yl-propan-2-ol); Bax inhibiting peptide P5 (also known as Bax inhibitor peptide P5); Kp7-6; FAIM(S) (Fas apoptosis inhibitory molecule-short); FAIM(L) (Fas apoptosis inhibitory molecule-long); Fas: Fc; FAP-1; NOK2; F2051; Fl926; F2928; ZB4; Fas M3 mAb; EGF; 740 Y-P; SC 3036 (KKHTDDGYMPMSPGVA) (SEQ ID NO:712); PI 3-kinase Activator (Santa Cruz Biotechnology, Inc.); Pam3Cys ((S)-(2,3-bis(palmitoyloxy)-(2RS)-propyl)-N-palmitoyl-(R)-Cys-(S)-Ser(S)-Lys4-OH, trihydrochloride); Act1 (NF-kB activator 1); an anti-DcB antibody; Acetyl-11-keto-b-Boswellic Acid; Andrographolide; Caffeic Acid Phenethyl Ester (CAPE); Gliotoxin; Isohelenin; NEMO-Binding Domain Binding Peptide (DRQIKIWFQNRRMKWKKTALDWSWLQTE) (SEQ ID NO:713); NF-kB Activation Inhibitor (6-Amino-4-(4-phenoxyphenylethylamino)quinazoline); NF-kB Activation Inhibitor II (4-Methyl-N1-(3-phenylpropyl)benzene-1,2-diamine); NF-kB Activation Inhibitor III (3-Chloro-4-nitro-N-(5-nitro-2-thiazolyl)-benzamide); NF-kB Activation hihibitor IV ((E)-2-Fluoro-4′-methoxystilbene); NF-kB Activation Inhibitor V (5-Hydroxy-(2,6-diisopropylphenyl)-1H-isoindole-1,3-dione); NF-kB SN50 (AA VALLP A VLLALLAPVQRKRQKLMP) (SEQ ID NO:714); Oridonin; Parthenolide; PPM-18 (2-Benzoylamino-1,4-naphthoquinone); Rol06-9920; Sulfasalazine; TIRAP Inhibitor Peptide (RQIKIWFNRRMKWKKLQLRDAAPGGAIVS) (SEQ ID NO:715); Withaferin A; Wogonin; BAY 11-7082 ((E)3-[(4-Methylphenyl)sulfonyl]-2-propenenitrile); BAY 11-7085 ((E)3-[(4-t-Butylphenyl)sulfonyl]-2-propenenitrile); (E)-Capsaicin; Aurothiomalate (ATM or AuTM); Evodiamine; Hypoestoxide; IKK Inhibitor III (BMS-345541); IKK Inhibitor VII; IKK Inhibitor X; IKK Inhibitor II; IKK-2 Inhibitor IV; IKK-2 Inhibitor V; IKK-2 Inhibitor VI; IKK-2 Inhibitor (SC-514); IkB Kinase Inhibitor Peptide; IKK-3 Inhibitor LX; ARRY-797 (Array BioPharma); SB-220025 (5-(2-Amino-4-pyrimidinyl)-4-(4-fluorophenyl)-1-(4-piperidinlyl)imidazole); SB-239063 (trans-4-[4-(4-Fluorophenyl)-5-(2-methoxy-4-pyrimidinyl)-1H-imidazol-1-yl]cyclohexanol); SB-202190 (4-(4-Fluorophenyl)-2-(4-hydroxyphenyl)-5-(4-pyridyl)1H-imidazole); JX-401 (-[2-Methoxy-4-(methylthio)benzoyl]-4-(phenylmethyl)piperidine); PD-169316 (4-(4-Fluorophenyl)-2-(4-nitrophenyl)-5-(4-ρyridyl)-1H-imidazole); SKF-86002 (6-(4-Fluorophenyl)-2,3-dihydro-5-(4-pyridinyl)imidazo[2,1-b]thiazole dihydrochloride); SB-200646 (N-(1-Methyl-1H-indol-5-yl)-N′-3-pyridinylurea); CMPD-I (2′-Fluoro-N-(4-hydroxyphenyl)-[1,l′-biphenyl]-4-butanamide); EO-1428 ((2-Methylphenyl)-[4-[(2-amino-4-bromophenyl)amino]-2-ch lorophenyl]methanone); SB-253080 (4-[5-(4-Fluorophenyl)-2-[4-(methylsulfonyl)phenyl]-1H-imidazol-4-yl]pyridine); SD-169 (1H-Indole-5-carboxamide); SB-203580 (4-(4- Fluorophenyl)-2-(4-methylsulfmyl phenyl)-5-(4-pyridyl) 1H-imidazole); TZP-101 (Tranzyme Pharma); TZP-102 (Tranzyme Pharma); GHRP-6 (growth hormone-releasing peptide-6); GHRP-2 (growth hormone-releasing peptide-2); EX-1314 (Elixir Pharmaceuticals); MK-677 (Merck); L-692,429 (Butanamide, 3-amino-3-methyl-N-(2,3,4,5-tetrahydro-2-oxo-1-((2′-(1H-tetrazol-5-yl)(1,1′-biphenyl)-4-yl)methyl)-1H-1-benzazepin-3-yl)-, (R)-); EP1572 (Aib-DTrp-DgTφ-CHO); diltiazem; metabolites of diltiazem; BRE (Brain and Reproductive organ-Expressed protein); verapamil; nimodipine; diltiazem; omega-conotoxin; GVIA; amlodipine; felodipine; lacidipine; mibefradil; NPPB (5-Nitro-2-(3-phenylpropylamino)benzoic Acid); flunarizine; erythropoietin; piperine; hemin; brazilin; z-V AD-FMK (Benzyloxycarbonyl-Val-Ala-Asp(OMe)-fluoromethylketone); Z-LEHD-FMK (benzyloxycarbonyl-Leu-Glu(OMe)-His-Asp(OMe)-fluoromethylketone); B-D-FMK (boc-aspartyl(Ome)-fluoromethylketone); Ac-LEHD-CHO (N-acetyl-Leu-Glu-His-Asp-CHO) (SEQ ID NO:716); Ac-IETD-CHO (N-acetyl-Ile-Glu-Thr-Asp-CHO) (SEQ ID NO:717); z-IETD-FMK (benzyloxycarbonyl-Ile-Glu(OMe)-Thr-Asp(OMe)-fluoromethy lketone); FAM-LEHD-FMK (benzyloxycarbonyl Leu-Glu-His-Asp-fluoromethyl ketone); FAM-LETD-FMK (benzyloxycarbonyl Leu-Glu-Thr-Asp-fluoromethyl ketone) (SEQ ID NO:721); Q-VD-OPH (Quinoline-Val-ASp-CH2-O-Ph); XIAP; cIAP-1; cIAP-2; ML-IAP; ILP-2; NAIP; Survivin; Brace; IAPL-3; fortilin; leupeptine; PD-150606 (3-(4-Iodophenyl)-2-mercapto-(Z)-2-propenoic acid); MDL-28170 (Z-Val-Phe-CHO); calpeptin; acetyl-calpastatin; MG 132 (N-[(phenylmethoxy)carbonyl]-L-leucyl-N-[(1S)-1-formyl-3 -methylbutyl]-L-leucinamide); MYODUR; BN 82270 (Ipsen); BN 2204 (Ipsen); AHLi-11 (Quark Pharmaceuticals), an mdm2 protein, pifithrin-α (1-(4-Methylphenyl)-2-(4,5,6,7-tetrahydro-2-imino-3(2H)-benzothiazolyl)ethanone); trans-stilbene; cis-stilbene; resveratrol; piceatannol; rhapontin; deoxyrhapontin; butein; chalcon; isoliquirtigen; butein; 4,2′,4′-trihydroxychalcone; 3,4,2′,4′,6′-pentahydroxychalcone; flavone; morin; fisetin; luteolin; quercetin; kaempferol; apigenin; gossypetin; myricetin; 6-hydroxyapigenin; 5-hydroxyflavone; 5,7,3′,4′,5′-pentahydroxyflavone; 3,7,3′,4′,5′-pentahydroxyflavone; 3,6,3′,4′-tetrahydroxyflavone; 7,3′,4′,5′-tetrahydroxyflavone; 3,6,2′,4′-tetrahydroxyflavone; 7,4′-dihydroxyflavone; 7,8,3′,4′-tetrahydroxyflavone; 3,6,2′,3′-tetrahydroxyflavone; 4′-hydroxyflavone; 5-hydroxyflavone; 5,4′-dihydroxyflavone; 5,7-dihydroxyflavone; daidzein; genistein; naringenin; flavanone; 3,5,7,3′,4′-pentahydroxyflavanone; pelargonidin chloride; cyanidin chloride; delphinidin chloride; (−)-epicatechin (Hydroxy Sites: 3,5,7,3′,4); (−)-catechin (Hydroxy Sites: 3,5,7,3′,4); (−)-gallocatechin (Hydroxy Sites: 3,5,7,3′,4′,5) (+)-catechin (Hydroxy Sites: 3,5,7,3′,4); (+)-epicatechin (Hydroxy Sites: 3,5,7,3′,4); Hinokitiol (b-Thujaplicin; 2-hydroxy-4-isopropyl-2,4,6-cycloheptatrien-1-one); L-(+)-Ergothioneine ((S)-a-Carboxy-2,3-dihydro-N,N,N-trimethyl-2-thioxo-1H-iniidazole4-ethanaminium inner salt); Caffeic Acid Phenyl Ester; MCI-186 (3-Methyl-1-phenyl-2-pyrazolin-5-one); HBED (N,N′-Di-(2-hydroxybenzyl)ethylenediamine-N,N′-diacetic acid*H2O); Ambroxol (trans-4-(2-Amino-3,5-dibromobenzylamino)cyclohexane-HCl; and U-83836E ((-)-2-((4-(2,6-di-l-Pyrrolidinyl-4-pyrimidinyl)-1-piperzainyl)methyl)-3,4-dihydro-2,5,7,8-tetramethyl-2H-1-benzopyran-6-ol>2HCl); B-1-5-methyl-nicotinamide-2′-deoxyribose; /3-D-1′-5-methyl-nico-tinamide-2′-deoxyribofuranoside; /3-1′-4,5-dimethyl-nicotinamide-2′-de-oxyribose; /3-D-1′-4,5-dimethyl-nicotmamide-2′-deoxyribofuranoside; 1-Naphthyl PPI (1-(1,1-Dimethylethyl)-3-(1-naphthalenyl)-1H-pyrazolo[3,4-d]pyrimidin-4-amine); Lavendustin A (5-[[(2,5-Dihydroxyphenyl)methyl][(2-hydroxyphenyl)methyl]amino]-2-hydroxybenzoic acid); MNS (3,4-Methylenedioxy-b-nitrostyrene); PPI (1-(1,1-Dimethylethyl)-1-(4-methylphenyl)-1H-pyrazolo[3, 4-d]pyrimidin-4-amine); PP2 (3-(4-chlorophenyl) 1-(1,1-dimethylethyl)-1H-pyrazolo[3,4-d]pyrimidin-4-amine); KX1-004 (Kinex); KX1-005 (Kinex); KX1-136 (Kinex); KX1-174 (Kinex); KX1-141 (Kinex); KX2-328 (Kinex); KX1-306 (Kinex); KX1-329 (Kinex); KX2-391 (Kinex); KX2-377 (Kinex); ZD4190 (Astra Zeneca; N-(4-bromo-2-fluorophenyl)-6-methoxy-7-(2-(1H-1,2,3-triazol-1-yl)ethoxy)quinazolin-4-amine); AP22408 (Ariad Pharmaceuticals); AP23236 (Ariad Pharmaceuticals); AP23451 (Ariad Pharmaceuticals); AP23464 (Ariad Pharmaceuticals); AZD0530 (Astra Zeneca); AZM475271 (M475271; Astra Zeneca); Dasatinib (N-(2-chloro-6-methylphneyl)-2-(6-(4-(2-hydroxyethyl)-piperazin-1-yl)-2-methylpyrimidin-4-ylamino) thiazole-5-carboxamide); GN963 (trans-4-(6,7-dimethoxyquinoxalin-2ylamino)cyclohexanol sulfate); Bosutinib (4-((2,4-dichloro-5-methoxyphenyl)amino)-6-methoxy-7-(3-(4-methyl-1-piperazinyl)propoxy)-3-quinolinecarbonitrile); or combinations thereof.

In some embodiments, the drug is an agent that reduces undesired neuron or nerve impulses. In some embodiments, the drug reduces one or more symptoms of dyskinesia or synkinesia. In some embodiments, the drug is carbamazepine, oxcarbazepine, phenytein, valproic acid, sodium valproate, cinnarizine, flunarizine, or nimodipine, or combinations thereof.

In some embodiments, the drug is an agent that promotes regeneration of neuron or nerve tissue. In some embodiments, the drug is a growth factor. In some embodiments, the drug is selected from brain-derived neurotrophic factor (BDNF); ciliary neurotrophic factor (CNTF); glial cell-line derived neurotrophic factor (GDNF); neurotrophin-3; neurotrophin-4; fibroblast growth factor (FGF) receptor; insulin-like growth factor (IGF); or a combination thereof.

Disclosed herein, in certain embodiments, are methods of delivering a photosensitizing agent to a human neuron or nerve comprising: contacting the human neuron or nerve with a nerve targeting peptide conjugate comprising (a) a nerve targeting peptide comprising the amino acid sequence of any one of SEQ ID NOS: 1-706, and (b) a photosensitizing agent. In some embodiments, the method further comprises exposing the human neuron or nerve with a light source that activates the photosensitizing agent, wherein the activated photosensitizing agent induces ablation or killing of the human neuron or nerve. Upon exposure to a specific wavelength of light, a photosensitizing agent reacts with molecular oxygen to produce singlet oxygen, which is cytotoxic. In certain embodiments, a photosensitizing agent is a porphyrin, chlorin, or dye. Examples of photosensitizing agents include porphyrin, protoporfin IX, purlytin, verteporfin, HPPH, temoporfin, methylene blue, photofrin, protofrin, hematoporphyrin, Talaporfin, benzopophyrin derivative monoacid, 5-aminileuvolinic acid, Lutetium texaphyrin, metallophthalocyanine, metallo-naphthocyaninesulfobenzo-porphyrazines, metallo-naphthalocyanines, zinc tetrasulfophthalocyanine, bacteriochlorins, metallochlorins, chlorine derivative, Tetra(m-hydroxyphenyl)chlorin (mTHPC), pheophorbide, dibromofluorescein (DBF), IR700DX, naphthalocyanine, and porphyrin derivatives.

Human neuron or nerve targeting molecules comprising a photosensitizing agent as disclosed herein can be used in methods of localized nerve killing in a subject. In some embodiments, human neuron or nerve targeting molecules comprising a photosensitizing agent are used for treating chronic pain (e.g., back, neck, or joint pain) in subject. In some embodiments, human neuron or nerve targeting molecules comprising a photosensitizing agent are used for treating prostate cancer in a subject. Autonomic innervation may contribute to prostate cancer growth and metastasis by light induced ablation of local autonomic nerves. Thus local autonomic nerves may be a viable target for prostate cancer therapy. In some embodiments, human neuron or nerve targeting molecules comprising a photosensitizing agent are used for treating renovascular hypertension in a subject by light induced ablation of sympathetic nerves in the renal vessels. In some embodiments, human neuron or nerve targeting molecules comprising a photosensitizing agent are used for treating excessive sweating. In some embodiments, human neuron or nerve targeting molecules comprising a photosensitizing agent are used for treating cardiac arrhythmias. In some embodiments, human neuron or nerve targeting molecules comprising a photosensitizing agent are used for treating pathological muscle spasms (e.g., Meige syndrome, hemifacial spasm, torticollis).

In some embodiments, a first nerve targeting peptide conjugate is administered in combination (simultaneously, concurrently, or serially) with a second nerve targeting peptide conjugate. In yet further embodiments, the cargo molecule of the first nerve targeting peptide conjugate and the cargo molecule of the second nerve targeting peptide conjugate are each independently selected from a fluorescently moiety, drug, photosensitizing agent, neurotrophic factor, and radiolabel. In some embodiments, the cargo molecule of the first nerve targeting peptide conjugate and the cargo molecule of the second nerve targeting peptide conjugate are the same or different.

### EXAMPLES

### Example 1: Phage Display Library Screening—Power B1 Analysis

Phage display library screens were used to identify peptides that bind to target proteins that are expressed in human nerves and could therefore be useful for systemic in-vivo labeling of nerves during fluorescence assisted surgery. Specifically, m13 phage libraries expressing 7 or 12 random amino acid peptides, or a cyclic peptide CX7C, on the N-terminus of the minor coat protein pIII (New England Biolabs) were processed through multiple rounds of selection for binding to target proteins expressed in peripheral nerve (Table 1).

TABLE 1

| | Protein | Source |
|---|---|---|
| A | Human MPZ | 1. Novoprotein NBL1 1321 Lysate- |
| | | adult normal |
| | | 2. Bon Opus Biosciences- P0/Mpz- |
| | | Recombinant protein |
| B | Human Myelin PLP | Novus NBP2-04617 Lysate-adult |
| | | normal |
| C | Human Nidogen-2 | Novus NBP2-10365-lysate normal |
| | | adult |
| D | Human Myelin | Enzo 200-606-M001 from human |
| | Basic Protein | brain |

The library was processed through up to 4 binding and wash cycles. For selection, phage libraries were incubated for up to 24 hours at 4° C. in 96 well plates that had been coated with one of the target proteins. Plates were then washed with PBS and phage were eluted using 0.2M glycine pH 2.0. Phage supernatants where then neutralized with Tris pH 9.0 prior to plating on TG1 bacteria and LB agar plates. Colonies were counted to determine titer followed by DNA preparation of pools of phage and NextGen sequencing. After rounds of selection, phage were pooled and amplified for iterative selection. Phage that were bound at each round were sequenced. NextGen sequencing of pools after 2, 3 and 4 rounds of selection was used to isolate candidate sequences that were analyzed further to identify those that specifically bound each unique target protein.

DNA sequences were trimmed of all vector amino acids and imported as a peptide dataset into Power BI. Power BI was used to identify phage display sequences for specific columns (corresponding to each target protein) in order to identify sequences that were distinct for each protein target. The Power BI code evaluated each individual sequence for each protein target against the sequences present in other protein targets in a test for uniqueness. Using the distinct flag, the Power BI code generated a filtered peptide dataset that excluded any peptide sequence that occur in more than one column which identified a specific protein target. This method therefore identified candidate peptide sequences showing selective binding to an individual target protein. Unique sequences in each column were sorted by individual counts such that the most selected sequences appeared at the top of the column. Tables 2A-2D list the top-ranked peptide sequences that, based on this analysis, showed selective binding to MPZ, MBP, PLP, and Nidogen-2, respectively.

TABLE 2A

| R3 Human MPZ | Count | SEQ ID NO:# |
|---|---|---|
| ALPLSAS | 196 | 1 |
| TSPWELR | 107 | 2 |
| SHLASRP | 97 | 3 |
| DPITRLK | 92 | 4 |
| VLKNNPT | 86 | 5 |
| IKPPYTH | 78 | 6 |
| ETTRNYG | 68 | 7 |
| SHSPGNK | 67 | 8 |
| WPSISRP | 57 | 9 |
| LSSPLSL | 56 | 10 |
| VLTKPMP | 56 | 11 |
| TMASPAK | 52 | 12 |
| KMAGHTV | 51 | 13 |
| SAHAKHH | 51 | 14 |
| DRNSVFW | 50 | 15 |
| GPLRMAT | 50 | 16 |
| TESLFPH | 50 | 17 |
| RVPHSRT | 49 | 18 |
| GLTPKLF | 48 | 19 |
| GDERIVRTLSHN | 47 | 20 |

TABLE 2B

| R3 Myelin Basic Protein | Count | SEQ ID NO:# |
|---|---|---|
| NDTKTPS | 19 | 21 |
| HPSTWHK | 18 | 22 |
| EHGYYKV | 16 | 23 |
| LPMRILT | 16 | 24 |
| SVLRMLN | 16 | 25 |
| AITSRNA | 15 | 26 |
| SKSDAWR | 14 | 27 |
| ILLPSLN | 13 | 28 |
| WTMKPNY | 13 | 29 |
| AGQLMRCANC | 12 | 30 |
| AWSTSSV | 12 | 31 |
| LRPISHE | 12 | 32 |
| MQTQGRV | 12 | 33 |
| SPDRTSL | 12 | 34 |
| DPWVMLR | 11 | 35 |
| QIGVLPS | 11 | 36 |
| QNTGLKW | 11 | 37 |
| AGFPPNT | 10 | 38 |
| DGKNTSN | 10 | 39 |
| HRLPWHH | 10 | 40 |

TABLE 2C

| R3 Myelin PLP | Count | SEQ ID NO:# |
|---|---|---|
| ATLKPYR | 68 | 41 |
| GLIRHYT | 66 | 42 |
| MTPHTSC | 62 | 43 |
| TNPWKPH | 62 | 44 |
| HQGSRTY | 53 | 45 |
| RFELPAP | 52 | 46 |
| YTPMGTG | 50 | 47 |
| GPFLFTV | 47 | 48 |
| LALYARM | 44 | 49 |
| LRFQIPP | 44 | 50 |
| FPVVTRN | 42 | 51 |
| RHNVEFS | 42 | 52 |
| LTHHFAP | 40 | 53 |
| NPSHHPR | 37 | 54 |
| YPKLWTY | 35 | 55 |
| FKMPIPT | 34 | 56 |
| NPNWRTI | 34 | 57 |
| SMWSQFR | 34 | 58 |
| DTSRTME | 33 | 59 |
| EKPSWTR | 33 | 60 |

TABLE 2D

| R3 Nidogen-2 | Count | SEQ ID NO:# |
|---|---|---|
| ACGEGEADVC | 26 | 61 |
| ACPENKSKHC | 13 | 62 |
| ACEPRSLANC | 11 | 63 |
| ACTVHKWDNC | 11 | 64 |
| GYWRNAL | 11 | 65 |
| KAIHPMRG | 11 | 66 |

### Example 2: Phage Display Library Screening—Python Script Analysis

Phage display library screens were used to identify peptides that bind to target proteins (including specific laminin trimers) that are expressed in human nerves, as described in Table 3. These screens were also carried out to identify peptides that could bind selectively to nerves versus muscle. Such peptides could therefore be useful for systemic in-vivo labeling of nerves during fluorescence assisted surgery. Specifically, m 13 phage libraries expressing 7 or 12 random amino acid peptides, or a cyclic peptide CX7C, on the N-terminus of the minor coat protein pIII (New England Biolabs; NEB) were processed through multiple rounds of selection for binding to target proteins expressed in nerves, or to excised human nerves, nerve extracts, or muscle, as described further below.

TABLE 3

| | Protein | Source |
|---|---|---|
| A | Human MPZ | 1. Novoprotein NBL1 1321 Lysate- |
| | | adult normal |
| | | 2. Bon Opus Biosciences- P0/Mpz- |
| | | Recombinant protein |
| B | Human Myelin | Enzo 200-606-M001 from human |
| | Basic Protein | brain |
| C | Human Myelin PLP | Novus NBP2-04617 Lysate-adult |
| | | normal |
| D | Human Nidogen-2 | Novus NBP2-10365-lysate normal |
| | | adult |
| E | Human Laminin Protein | Biolamina human recombinant |
| | | laminin 211 (LN211), 411 (LN411), |
| | | 421 (LN421), and 521 (LN521) |

### Screening

### Target Protein Screening

For screening against the specified target proteins in Table 1, including LN211, LN411, LN421, and LN521, the phage library was processed by up to 4 binding and wash cycles. For selection, phage libraries were incubated for up to 24 hours at 4° C. in 96 well plates that had been coated with one of the target proteins. Plates were then washed with PBS and phage were eluted using 0.2M glycine pH 2.0. Phage supernatants where then neutralized with Tris pH 9.0 prior to plating on TGI bacteria and LB agar plates. Colonies were counted to determine titer followed by DNA preparation of pools of phage and NextGen sequencing. After rounds of selection, phage were pooled and amplified for iterative selection. Phage that were bound at each round were sequenced. NextGen sequencing of pools after 2, 3 and 4 rounds of selection was used to isolate candidate sequences that were analyzed further to identify those that specifically bound each unique target protein.

### Nerve and Muscle Tissue Screening

To identify peptides selective for nerves versus muscle, phage from the same libraries used for specific target proteins were used to screen against excised human nerve tissue (or nerve extracts) and human muscle tissue.

Excised Nerve Tissue: Phage were processed through multiple cycles of selection, with representative phage being isolated and sequenced after each selection cycle. Specifically, for selection using human nerve tissue, a sample of nerve muscle tissue (2 mm by 2 mm) was washed prior to mixing and incubation with a phage library. After incubation, the mixture (containing mostly intact nerves with phage particles that had variable affinity for nerves) was centrifuged and the nerve pellet washed multiple times with PBS to remove phage with little or no affinity for nerve tissue. The pellet was homogenized and plated for titering and reamplification. Phage that were bound at each round were sequenced. NextGen sequencing of pools after 2, 3 and 4 rounds of selection were used to isolate candidate sequences that were further analyzed using bioinformatics.

Nerve Extracts: Phage libraries were incubated for up to 24 hours at 4° C. in 96 well plates that had been coated with a human nerve extract. Plates were then washed with PBS and phage were eluted using 0.2M glycine pH 2.0. Phage supernatants where then neutralized with Tris pH 9.0 prior to plating on TG1 bacteria and LB agar plates. Colonies were counted to determine titer followed by DNA preparation of pools of phage and NextGen sequencing. After rounds of selection, phage were pooled and amplified for iterative selection. Phage that eluted by 0.2M glycine pH 2.0 (the nerve binders) at each round were sequenced. NextGen sequencing of pools after 2, 3 and 4 rounds of selection were used to isolate candidate sequences that were further analyzed using bioinformatics.

Muscle Tissue: Phage were processed through multiple cycles of selection, with representative phage being isolated and sequenced after each selection cycle. Specifically, for selection using human skeletal muscle tissue, a sample of skeletal muscle tissue (2 mm by 2 mm) was washed prior to mixing and incubation with a phage library. After incubation, the mixture (containing mostly intact muscle with phage particles that had variable affinity for muscle) was centrifuged and the muscle pellet washed multiple times with PBS to remove phage with little or no affinity for muscle tissue. The pellet was then homogenized and plated for titering and reamplification. Phage that were bound at each round were sequenced. NextGen sequencing of pools after 2, 3 and 4 rounds of selection were used to isolate candidate sequences that were further analyzed using bioinformatics.

### Sequence Analysis

### NEB Nerve Protein-Binding Peptides

Python Script Analysis: For all NEB sequence datasets, a python script was used to translate, trim, and filter the peptide sequences. The script translated the nucleotide sequences, translating amber codons as glutamine in peptide sequences, and then trimmed the end sequences that come from the vector. Secondly, the script combined the unique counts of trimmed peptides with identical sequences. Finally, the script filtered out peptide sequences that were likely artifacts or otherwise not useful: (1) peptides that had incorrect lengths for the libraries used (correct are 7, 9, or 12 amino acids long); (2) peptides that had a single or an odd number of Cysteines; (3) peptides that were truncated by stop codons (except the amber stop); and (4) unique peptide sequences that occurred less than six times in a dataset. Another python script was used to group peptides from different assays according to protein target (Myelin Protein Zero, Myelin Proteolipid Protein, Myelin Basic Protein, and Nidogen-2) and also combine the unique sequence counts for each peptide. An R script was written to merge the peptide sequences from different data files and determine peptides specific for each protein target but absent from different protein binding datasets. Peptide sequences were ranked by unique sequence count.

Tables 4A-4D list the top-ranked peptide sequences, based on these analyses, that showed selective binding to MPZ, MBP, PLP, and Nidogen-2, respectively. This analysis yielded some sequences that were previously identified in Example 1, and these are indicated by the corresponding original SEQ ID NOs from Example 1.

### NEB Laminin-Binding Peptides

For all NEB sequence datasets, a python script was used to translate, trim, and filter the peptide sequences (see details above). A python script was used to group peptides from different assays according to laminin complex binding (laminin complex 211, 411, 421, or 521) and combine the unique sequence counts for each peptide. An R script was written to merge the peptide sequences from different data files and determine peptides that specifically bind one laminin complex and not the others, or that bind both laminin 421 and 521 but not 211 and 411.

Table 4E shows the top ranked peptide sequences, based on this analysis, showing selective binding to laminin trimers 421, 521, or both, as these forms can show preferential expression in nerve tissue.

### NEB Tissue-Binding Peptides

For all NEB sequence datasets, a python script was used to translate, trim, and filter the peptide sequences (see details above). A python script was used to group peptides from different assays according to tissue target (muscle, nerve, neuron, or nerve extract) and combine the unique sequence counts for each peptide. An R script was written to merge the peptide sequences from different data files and determine peptides that bind both neuron and nerve extracts but not muscle extracts. Peptide sequences were ranked by unique sequence count.

Table 4F shows the top ranked peptide sequences, based on this analysis, showing selective binding to nerves (compared to muscle).

TABLE 4A

| Human MPZ | Count | SEQ ID NO: |
|---|---|---|
| CLKQQTGDC | 37 | 67 |
| EGHVWSEYTWGT | 37 | 68 |
| FMSDVDH | 37 | 69 |
| ISSVTVV | 37 | 70 |
| KQPAMEN | 37 | 71 |
| LISPTAR | 37 | 72 |
| LVLQTTP | 37 | 73 |
| NPTSEGA | 37 | 74 |
| THSTDLS | 37 | 75 |
| TTNVTEIERESY | 37 | 76 |
| YAPWRVF | 37 | 77 |
| YGQSITE | 37 | 78 |
| CDIKDANSC | 38 | 79 |
| CIKGEDNQC | 38 | 80 |
| HNQLLYT | 38 | 81 |
| IEQVGWR | 38 | 82 |
| KTPILAS | 38 | 83 |
| LWAKRDA | 38 | 84 |
| NPISPRN | 38 | 85 |
| NTASMTT | 38 | 86 |
| SAHSVVE | 38 | 87 |
| YSSLTSN | 38 | 88 |
| AWTMRAS | 39 | 89 |
| CERIGPREC | 39 | 90 |
| CQKWFTFAC | 39 | 91 |
| CSNALNKAC | 39 | 92 |
| FSPRDVS | 39 | 93 |
| FSTTGRG | 39 | 94 |
| HHPLFID | 39 | 95 |
| LVNPFPE | 39 | 96 |
| YEGERGA | 39 | 97 |
| CEDRQRSMC | 40 | 98 |
| CGEKIMSLC | 40 | 99 |
| DRYLLNQ | 40 | 100 |
| FPSYTIS | 40 | 101 |
| GGSKVWG | 40 | 102 |
| GLWRHTM | 40 | 103 |
| KRNLWDI | 40 | 104 |
| SPNTNSH | 40 | 105 |
| TLLHYSA | 40 | 106 |
| VPLAYLR | 40 | 107 |
| VVPLKWI | 40 | 108 |
| CRVGVTSAC | 41 | 109 |
| FALPAGL | 41 | 110 |
| FRVTQTA | 41 | 111 |
| HPLMVPS | 41 | 112 |
| LPNANNL | 41 | 113 |
| MIHTRQT | 41 | 114 |
| SETESYM | 41 | 115 |
| SNALSYF | 41 | 116 |
| SYLPPFI | 41 | 117 |
| VHTPYRS | 41 | 118 |
| WPRPIQI | 41 | 119 |
| WTSVATA | 41 | 120 |
| ALPISYL | 42 | 121 |
| FEHMAVT | 42 | 122 |
| NASVPPK | 42 | 123 |
| SGHGAFR | 42 | 124 |
| TTLVVTA | 42 | 125 |
| CPDAARNSC | 43 | 126 |
| CYSSVADMC | 43 | 127 |
| LSPPRIM | 43 | 128 |
| WMTNLDP | 43 | 129 |
| KLPTLSV | 44 | 130 |
| AMSNLSY | 45 | 131 |
| HPFPRFD | 45 | 132 |
| LPAESHW | 45 | 133 |
| STHEWRT | 45 | 134 |
| TTWPNTA | 45 | 135 |
| YMKHSPG | 45 | 136 |
| AYLEDWRSMSTR | 46 | 137 |
| GPVVSGM | 46 | 138 |
| HLDSRRH | 46 | 139 |
| LVLSNPR | 46 | 140 |
| NMSHSNR | 46 | 141 |
| SVAYVPV | 46 | 142 |
| YPQARTS | 46 | 143 |
| MYHDSVS | 47 | 144 |
| ADNVLRRALENI | 49 | 145 |
| DRNSVFW | 50 | 15 |
| QIAHLEY | 50 | 146 |
| GMVSPHHSVYRH | 51 | 147 |
| GPLRMAT | 51 | 16 |
| GSWSSGF | 52 | 148 |
| SAHAKHH | 52 | 14 |
| TESLFPH | 52 | 17 |
| NGAYSLAIRYTS | 53 | 149 |
| KMAGHTV | 54 | 13 |
| LSSPLSL | 56 | 10 |
| VLTKPMP | 56 | 11 |
| TMASPAK | 58 | 12 |
| WPSISRP | 58 | 9 |
| YTPNWHFRWMPA | 60 | 150 |
| GEWRARIDQDVS | 65 | 151 |
| ETTRNYG | 69 | 7 |
| SHSPGNK | 79 | 8 |
| IKPPYTH | 82 | 6 |
| VLKNNPT | 87 | 5 |
| DPITRLK | 93 | 4 |
| TSPWELR | 108 | 2 |
| ALPLSAS | 197 | 1 |

TABLE 4B

| R3 Myelin Basic Protein | Count | SEQ ID NO: |
|---|---|---|
| AGSLLSL | 6 | 152 |
| AIPPRKL | 6 | 153 |
| AISWKGF | 6 | 154 |
| AKFTMWV | 6 | 155 |
| ALRDTRV | 6 | 156 |
| ALYNQMT | 6 | 157 |
| AQFLTIY | 6 | 158 |
| AQLGAFR | 6 | 159 |
| ARVPNPL | 6 | 160 |
| CTMTNQYDC | 6 | 161 |
| DSQEKSV | 6 | 162 |
| DVRIWTL | 6 | 163 |
| ELKSLRF | 6 | 164 |
| ESKSPRE | 6 | 165 |
| ETHYKIA | 6 | 166 |
| FDLLARK | 6 | 167 |
| FQLSYET | 6 | 168 |
| GEPALVT | 6 | 169 |
| GPRVTPH | 6 | 170 |
| GQWWKML | 6 | 141 |
| GRHTDVV | 6 | 172 |
| GSPYGWG | 6 | 173 |
| GVAEVGL | 6 | 174 |
| HAHWARL | 6 | 175 |
| HFTFFPI | 6 | 176 |
| HPWSHPN | 6 | 177 |
| HQMRTML | 6 | 178 |
| HQPLFPR | 6 | 179 |
| HRTHLHQ | 6 | 180 |
| HSGPLLP | 6 | 181 |
| HSSATMS | 6 | 182 |
| IGTRWHQ | 6 | 183 |
| INFPFAV | 6 | 184 |
| IPIPYRT | 6 | 185 |
| IPYSQMP | 6 | 186 |
| KIITTKS | 6 | 187 |
| KMSLAPP | 6 | 188 |
| KVHHLAR | 6 | 189 |
| LELHQNV | 6 | 190 |
| LITHPQL | 6 | 191 |
| LMLPQLE | 6 | 192 |
| LVPPVNH | 6 | 193 |
| MNSPLAQ | 6 | 194 |
| MQRLGAD | 6 | 195 |
| NGPNSLQ | 6 | 196 |
| NGVSSSL | 6 | 197 |
| NLTYLRF | 6 | 198 |
| QGAYVRP | 6 | 199 |
| QKALPQT | 6 | 200 |
| QTSIMSG | 6 | 201 |
| QYWDILG | 6 | 202 |
| RGHPSQL | 6 | 203 |
| RSRKHRP | 6 | 204 |
| RTTDWWT | 6 | 205 |
| RVIPSTT | 6 | 206 |
| SAVLMPP | 6 | 207 |
| SEGLTLL | 6 | 208 |
| SGPFPLV | 6 | 209 |
| SIQTSAM | 6 | 210 |
| SKLAGFP | 6 | 211 |
| SKPHASH | 6 | 212 |
| SLDHLRL | 6 | 213 |
| SLKTTNP | 6 | 214 |
| SLWQTLR | 6 | 215 |
| SLWTHRT | 6 | 216 |
| SNAHTLP | 6 | 217 |
| SPKPDIP | 6 | 218 |
| SRHLPPL | 6 | 219 |
| SSFYVAY | 6 | 220 |
| SSMRYNS | 6 | 221 |
| SVGLPTK | 6 | 222 |
| SYRSMAP | 6 | 223 |
| SYVTVFR | 6 | 224 |
| TGPSSAV | 6 | 225 |
| TGSVSKQ | 6 | 226 |
| THKHMMT | 6 | 227 |
| TIRVAHT | 6 | 228 |
| TPGRATL | 6 | 229 |
| TPVLWLP | 6 | 230 |
| TPVRLNT | 6 | 231 |
| TRIPILL | 6 | 232 |
| TTTQGHP | 6 | 233 |
| VGTFWTR | 6 | 234 |
| VIRTAAM | 6 | 235 |
| VLSPYHN | 6 | 236 |
| VRGTPNH | 6 | 237 |
| WDLPQNR | 6 | 238 |
| WDLRHSK | 6 | 239 |
| WTYHPTT | 6 | 240 |
| WVPDRSL | 6 | 241 |
| YPTYLSY | 6 | 242 |
| YPYVPIY | 6 | 243 |
| YSSRLND | 6 | 244 |
| YTASPRW | 6 | 245 |
| YVNRVKQ | 6 | 246 |
| AAGQRSF | 7 | 247 |
| AILTRPP | 7 | 248 |
| ALGTRPL | 7 | 249 |
| ASNTSKG | 7 | 250 |
| AVRQTTM | 7 | 251 |
| CEDSDKSVC | 7 | 252 |
| CNRVPFKQC | 7 | 253 |
| CPRMNNPLC | 7 | 254 |
| DVVKDVI | 7 | 255 |
| ESPYPHS | 7 | 256 |
| FKLPWAS | 7 | 257 |
| FLNQAPT | 7 | 258 |
| FSAGVGK | 7 | 259 |
| GHSHYILGKPTA | 7 | 260 |
| GPITHAI | 7 | 26 |
| HFLRPSD | 7 | 262 |
| HLLKTHS | 7 | 263 |
| HMAGVNQ | 7 | 264 |
| HPDTLPQ | 7 | 265 |
| HPTGTPT | 7 | 266 |
| HTGLRSL | 7 | 267 |
| KGQPLFR | 7 | 268 |
| KYRHGAE | 7 | 269 |
| LFRRVTE | 7 | 270 |
| LTKSLNH | 7 | 271 |
| MPLLELP | 7 | 272 |
| MTNHGNA | 7 | 273 |
| NLKLWTR | 7 | 274 |
| NLLQGVM | 7 | 275 |
| NMYPRVT | 7 | 276 |
| NSSPHQI | 7 | 277 |
| QGTLFDT | 7 | 278 |
| QPIDSTS | 7 | 279 |
| SEPGTVR | 7 | 280 |
| SGNLKKA | 7 | 281 |
| SIAEVLGLWRNV | 7 | 282 |
| SIRMTEI | 7 | 283 |
| SISKIRT | 7 | 284 |
| SLRSVGA | 7 | 285 |
| SSGMLSK | 7 | 286 |
| SYTLSRS | 7 | 287 |
| TLLTALT | 7 | 288 |
| TPLYLSS | 7 | 289 |
| VSFTLEP | 7 | 290 |
| VVDMSTY | 7 | 291 |
| WPDLRIL | 7 | 292 |
| WSLPLLS | 7 | 293 |
| WSPRWPS | 7 | 294 |
| YLPPPLP | 7 | 295 |
| DSYLLSA | 8 | 296 |
| FVLPNKN | 8 | 297 |
| IILPSAQ | 8 | 298 |
| KNSIAPR | 8 | 299 |
| LPPQISR | 8 | 300 |
| MPSLKHQ | 8 | 301 |
| NPTDTNK | 8 | 302 |
| NTSLSFK | 8 | 303 |
| QNSYLSN | 8 | 304 |
| QQAHLQS | 8 | 305 |
| SPPRFIP | 8 | 306 |
| SWFESHN | 8 | 307 |
| TPQVMLK | 8 | 308 |
| TSPPLAH | 8 | 309 |
| TSRLVST | 8 | 310 |
| VAHQRVS | 8 | 311 |
| VPTLRIP | 8 | 312 |
| YATATPS | 8 | 313 |
| ANLSRSV | 9 | 314 |
| FSARTNT | 9 | 315 |
| HFYGPGP | 9 | 316 |
| HPWLGNR | 9 | 317 |
| HYSPNVM | 9 | 318 |
| NFAQHMQ | 9 | 319 |
| SNAERWR | 9 | 320 |
| SRPTRVP | 9 | 321 |
| SVEYGQL | 9 | 322 |
| TGNLRLY | 9 | 323 |
| HRLPWHH | 10 | 40 |
| MDLSLKP | 10 | 324 |
| NTPKVLA | 10 | 325 |
| TQMTMDS | 10 | 326 |
| DPWVMLR | 11 | 35 |
| QNTGLKW | 11 | 37 |
| AWSTSSV | 12 | 31 |
| SPDRTSL | 12 | 34 |
| WTMKPNY | 14 | 29 |
| LPMRILT | 16 | 24 |
| EHGYYKV | 17 | 23 |

TABLE 4C

| Myelin PLP | Count | SEQ ID NO: |
|---|---|---|
| ASLTLAL | 26 | 327 |
| DRHEGHR | 26 | 328 |
| FRIDKSM | 26 | 329 |
| GHLQSLF | 26 | 330 |
| GLWGPSL | 26 | 331 |
| KPSASIY | 26 | 332 |
| LPSHASI | 26 | 333 |
| LPVKNLL | 26 | 334 |
| MKIMPMD | 26 | 335 |
| SAQSRSF | 26 | 336 |
| SLPSLLP | 26 | 337 |
| SVVYQNS | 26 | 338 |
| TTTIADM | 26 | 339 |
| VTGPGTP | 26 | 340 |
| VTPNKAR | 26 | 341 |
| WHYTPSM | 26 | 342 |
| WSRISVD | 26 | 343 |
| ALFQERK | 27 | 344 |
| AQASSAR | 27 | 345 |
| EVLYPNN | 27 | 346 |
| HLHTRPT | 27 | 347 |
| IRPTHNG | 27 | 348 |
| KGVIPAT | 27 | 349 |
| LHQTYRP | 27 | 350 |
| LPHRLNP | 27 | 351 |
| TGISSTP | 27 | 352 |
| TMPIKAM | 27 | 353 |
| VPGHISG | 27 | 354 |
| VSASWMP | 27 | 355 |
| WSPHGYK | 27 | 356 |
| YSQAISA | 27 | 357 |
| AVVAMNK | 28 | 358 |
| GIPSSKN | 28 | 359 |
| NDASTVS | 28 | 360 |
| QLPRNNL | 28 | 361 |
| SLNRGGA | 28 | 362 |
| SLPGYRH | 28 | 363 |
| SVLPDKL | 28 | 364 |
| TLWAQKT | 28 | 365 |
| TQPRYPS | 28 | 366 |
| VFPERRV | 28 | 367 |
| VGYRSAS | 28 | 368 |
| VRTSMNH | 28 | 369 |
| YTWTPSR | 28 | 370 |
| FSLDRDG | 29 | 371 |
| FSTPTNV | 29 | 372 |
| GPAAVII | 29 | 373 |
| GWSTAIR | 29 | 374 |
| HKAPLGT | 29 | 375 |
| IDRVRGL | 29 | 376 |
| LMQKPSI | 29 | 377 |
| SVMWMTP | 29 | 378 |
| TKITPHR | 29 | 379 |
| YSSRLTA | 29 | 380 |
| ARFVPLT | 30 | 381 |
| DGSRDLV | 30 | 382 |
| EKSTTVA | 30 | 383 |
| YSDTYKH | 30 | 384 |
| EPSSFTF | 31 | 385 |
| GKSYSQI | 31 | 386 |
| GNVFQTS | 31 | 387 |
| IIPPTSV | 31 | 388 |
| LPLRLHA | 31 | 389 |
| NVFSATP | 31 | 390 |
| QRRSVIL | 31 | 391 |
| SGVATYT | 31 | 392 |
| SWDVLEL | 31 | 393 |
| TFPERLR | 31 | 394 |
| WSPHRSF | 31 | 395 |
| IHHLESS | 32 | 396 |
| LNGISAL | 32 | 397 |
| SALISTR | 32 | 398 |
| SGLHYAL | 32 | 399 |
| TRASYPQ | 32 | 400 |
| NIPSSIL | 33 | 401 |
| QPNMLKP | 33 | 402 |
| RLQPEPT | 33 | 403 |
| SVEKYSK | 33 | 404 |
| DTSRTME | 33 | 59 |
| EKPSWTR | 33 | 60 |
| VSSQEQA | 34 | 405 |
| WNTADRL | 34 | 406 |
| FKMPIPT | 34 | 56 |
| NPNWRTI | 34 | 57 |
| SMWSQFR | 34 | 58 |
| YPKLWTY | 35 | 55 |
| MPHAPVQ | 37 | 407 |
| NPSHHPR | 37 | 54 |
| TDVEMVP | 40 | 408 |
| LTHHFAP | 40 | 53 |
| AHQSSVT | 42 | 409 |
| RHNVEFS | 42 | 52 |
| GSAWKKH | 44 | 410 |
| LALYARM | 44 | 49 |
| LRFQIPP | 44 | 50 |
| YTPMGTG | 50 | 47 |
| RFELPAP | 52 | 46 |
| HQGSRTY | 53 | 45 |
| TNPWKPH | 62 | 44 |
| GLIRHYT | 66 | 42 |
| ATLKPYR | 68 | 41 |

TABLE 4D

| Nidogen-2 | Count | SEQ ID NO: |
|---|---|---|
| CIHHKGVLC | 6 | 411 |
| CKDGHIRHC | 6 | 412 |
| CNLATSLQC | 6 | 413 |
| CRSPHEPMC | 6 | 414 |
| CVDKMASVC | 6 | 415 |
| CYSAPTKSC | 6 | 416 |
| EAMMHRN | 6 | 417 |
| LHITPEV | 6 | 418 |
| LSIRGLT | 6 | 419 |
| NILSQVN | 6 | 420 |
| QLLESRT | 6 | 421 |
| QMDAKHM | 6 | 422 |
| QTLRPKQ | 6 | 423 |
| TIASLLV | 6 | 424 |
| WLAAGSQ | 6 | 425 |
| ALYLPGR | 7 | 426 |
| ARMAFSL | 7 | 427 |
| ATYNMSQ | 7 | 428 |
| GLRTMEP | 7 | 429 |
| TPWLPTI | 7 | 430 |
| YNHTMMY | 7 | 431 |
| CSFKMNQKC | 8 | 432 |
| LVHPFHG | 8 | 433 |
| CSSSAPRIC | 9 | 434 |
| HSRLPTP | 13 | 435 |

TABLE 4E

| Laminin 421/521 | Count | SEQ ID NO: |
|---|---|---|
| CKSPIKGTC | 27 | 436 |
| CLQKNHKFC | 27 | 437 |
| CVLKCEDQKYRG | 27 | 438 |
| DGANMENIAPAN | 27 | 439 |
| DRMYSTVPAEGL | 27 | 440 |
| EAVSTQI | 27 | 441 |
| GINLRALDLHAN | 27 | 442 |
| HQSVTGVRSHFH | 27 | 443 |
| SVVGWAAPRTAQ | 27 | 444 |
| TILKPAAQGFAD | 27 | 445 |
| TSAVSLR | 27 | 446 |
| TTLWLDRDEALK | 27 | 447 |
| VIADQSKSAVAV | 27 | 448 |
| AQNLRVHAWASL | 28 | 449 |
| CPMHQSKTC | 28 | 450 |
| CTDGRNFVC | 28 | 451 |
| CTGETLLTC | 28 | 452 |
| CTSSSHQTC | 28 | 453 |
| ETGGLAYGSGQK | 28 | 454 |
| GIVPSRHTTGLG | 28 | 455 |
| GWTSDLSRNVRG | 28 | 456 |
| HTQIHRL | 28 | 457 |
| KFADTKLTSLRY | 28 | 458 |
| QLQHVHL | 28 | 459 |
| QPTPSQIKFTRT | 28 | 460 |
| RALDRYLPWSPH | 28 | 461 |
| SLPYAASLNSVE | 28 | 462 |
| STNVQRA | 28 | 463 |
| SVLSTGTANQRH | 28 | 464 |
| SVQTPMWRSLVG | 28 | 465 |
| SWHFTGTPFMNR | 28 | 466 |
| SYTATWSEMSRS | 28 | 467 |
| TALSHRHEAMRW | 28 | 468 |
| TKGIAPK | 28 | 469 |
| TVARSTAQERSI | 28 | 470 |
| AVRPLGLPDNHR | 29 | 471 |
| CIGGPHRNC | 29 | 472 |
| CKSPAIKGC | 29 | 473 |
| CNSWKAAKC | 29 | 474 |
| DSLYRGMHQPRI | 29 | 475 |
| EDRMMTYRYTST | 29 | 476 |
| EVERILTPHVNN | 29 | 477 |
| FNSDSRSTHQED | 29 | 478 |
| HPAWADFFTMSS | 29 | 479 |
| IDLSLRS | 29 | 480 |
| IDQSGLQKSGMK | 29 | 481 |
| LGSSRSPSSFLG | 29 | 482 |
| NGHEVQSRAANR | 29 | 483 |
| QGWKDRLPIWRY | 29 | 484 |
| SHDVANGTSVRT | 29 | 485 |
| SPDRTNWGWQTN | 29 | 486 |
| SVESNSKLTMPR | 29 | 487 |
| WPMMQTR | 29 | 488 |
| CKSERGPEC | 30 | 489 |
| CQGWPRPMC | 30 | 490 |
| CSRAGLSAC | 30 | 491 |
| CTWKHRDNC | 30 | 492 |
| GLLDWGSLQGGN | 30 | 493 |
| GPLLFRG | 30 | 494 |
| GSPEKRT | 30 | 495 |
| KASIAYD | 30 | 496 |
| LGYGQGTPHRSN | 30 | 497 |
| QGWEFQVPARHS | 30 | 498 |
| QTFGYSMFNVRT | 30 | 499 |
| RNLHYPLNHPFM | 30 | 500 |
| TAPRWTQ | 30 | 501 |
| VPSYILR | 30 | 502 |
| WRSPLTT | 30 | 503 |
| CQTSGHHQC | 31 | 504 |
| CSSQAKRSC | 31 | 505 |
| CTAWTRQEC | 31 | 506 |
| EYVKFSHSPRTY | 31 | 507 |
| GGWQSLWDKPEH | 31 | 508 |
| GPMLKNLSDAVT | 31 | 509 |
| NHDQGSLTRWRS | 31 | 510 |
| SLLSVVNTSSKS | 31 | 511 |
| VVPSWPSVHRPP | 31 | 512 |
| WGWNGANMSPRG | 31 | 513 |
| CHNADNNGC | 32 | 514 |
| LPMLRHS | 32 | 515 |
| NDTGRHASGISK | 32 | 516 |
| QALRTNYSPLNS | 32 | 517 |
| TLKAGTRANDGV | 32 | 518 |
| CGETTAGKC | 33 | 519 |
| HSTYAPG | 33 | 520 |
| QINWKQADKNAD | 33 | 521 |
| ALPWQSGVHGTK | 34 | 522 |
| TFGSGRSMPIQY | 34 | 523 |
| VYRNGGGLPLTA | 34 | 524 |
| ALKIGPETTIYM | 35 | 525 |
| DWTQVRVTNWFL | 35 | 526 |
| LGHQNGGRADMW | 35 | 527 |
| MTNSGAK | 35 | 528 |
| WAYTDYM | 35 | 529 |
| AHAAGRDMRQGT | 36 | 530 |
| DVIHSSRGAYFE | 36 | 531 |
| NWTHLGVARLQP | 36 | 532 |
| SYDGTMLKQVRL | 36 | 533 |
| WQWRSTELGYRY | 36 | 534 |
| WSPANGWRHQTI | 36 | 535 |
| CLHNGQRSC | 37 | 536 |
| CTPRSATLC | 37 | 537 |
| DIMKSPRSNLRS | 37 | 538 |
| QTFHEYLNPARG | 37 | 539 |
| SLNTTWVSPMMK | 37 | 540 |
| AQRMNQA | 38 | 541 |
| GDAQILM | 38 | 542 |
| GHRTLVTSERYL | 38 | 543 |
| LGPSTQGAGQTR | 38 | 544 |
| TFAAAQAELIMV | 38 | 545 |
| WEEHRVEILPDV | 38 | 546 |
| AGAYTSRHAFDE | 39 | 547 |
| CGLATNKSC | 39 | 548 |
| GDVITAYINPWP | 39 | 549 |
| GYDLSRLWGMAS | 39 | 550 |
| ANRYLAS | 40 | 551 |
| CKWTQLWGC | 40 | 552 |
| CLKDTHLNC | 40 | 553 |
| GQPRNIHLPGTH | 40 | 554 |
| GSTTHPHFGLPG | 40 | 555 |
| GWIQDTFVLGRS | 40 | 556 |
| QGHPFIY | 40 | 557 |
| SNPKHVSSLGQM | 40 | 558 |
| WGANTTGRTHGG | 40 | 559 |
| AQGVWWSEWFAP | 42 | 560 |
| CFQPKMNSC | 42 | 561 |
| CWSGNSRSC | 42 | 562 |
| FPLKIRT | 42 | 563 |
| LARSSIMAANNV | 42 | 564 |
| AKTQAPSNWSGV | 43 | 565 |
| CSVWNSGNC | 43 | 566 |
| CTGRISKHC | 43 | 567 |
| GAMMNSNNLVAR | 43 | 568 |
| GGGYHIFGPLVT | 43 | 569 |
| HSFLGAR | 44 | 570 |
| LLAGPFR | 44 | 571 |
| QTLQTRH | 44 | 572 |
| ALGGEPRKAYQR | 45 | 573 |
| CNHLTQKLC | 45 | 574 |
| CTSKLARHC | 45 | 575 |
| AVYPLDLGAGMR | 46 | 576 |
| CHRTYNSTC | 46 | 577 |
| LDRNADSVRAVL | 46 | 578 |
| TTNGVPGHDRSP | 46 | 579 |
| ADMPQMTLKYGV | 47 | 580 |
| AWNGNRS | 47 | 581 |
| GDLLLKR | 47 | 582 |
| VPQIRIK | 47 | 583 |
| SWQMRSY | 48 | 584 |
| YHLGPNQKMRTS | 48 | 585 |
| ASHSRPLMNYAP | 49 | 586 |
| QTRWDDGSYQIS | 49 | 587 |
| SLAVANTRFMIR | 49 | 588 |
| EGHVWSEYTWGT | 50 | 589 |
| ETYKVTRVISPW | 50 | 590 |
| GHPWTEIDFMSS | 50 | 591 |
| KQELSDNLASHR | 50 | 592 |
| SFRNLEKLSLWS | 50 | 593 |
| GDLWNVP | 51 | 594 |
| GPKNHHQ | 51 | 595 |
| GSGFRNEEHSAH | 51 | 596 |
| NNNAAMQNHGVR | 51 | 597 |
| RAQAHQV | 51 | 598 |
| CSLANPATC | 52 | 599 |
| GEPLTRFPNSDS | 52 | 600 |
| KPILWNRSLNAL | 52 | 601 |
| GFADVIHRKWSS | 53 | 602 |
| HQSLAGVPWSRH | 53 | 603 |
| VPVFLDQARVMK | 53 | 604 |
| DDLLTAPRLGVW | 54 | 605 |
| SDRGKTVLHGQI | 54 | 606 |
| CGITQTTTC | 55 | 607 |
| CHQRQQTYC | 55 | 608 |
| EYWHQRGSWFHR | 55 | 609 |
| YTPRNQL | 55 | 610 |
| CTKSPSNSC | 58 | 611 |
| WAFKAPQ | 61 | 612 |
| GSLDMGNNKQPV | 62 | 613 |
| AVVSQNQMSQQK | 64 | 614 |
| CLNQKWEAC | 64 | 615 |
| WPVRNLL | 67 | 616 |
| GYRSFIHENWSI | 68 | 617 |
| SGWEGKTHQGVR | 69 | 618 |
| LQSNAADHHQGM | 75 | 619 |
| ATSKYPNSWAQT | 80 | 620 |
| DRLTPITWDWSR | 86 | 621 |
| WTKHNTPAHHLS | 89 | 622 |
| WNANYGTMTRHA | 92 | 623 |
| HASNQRV | 93 | 624 |
| GRTIHSM | 95 | 625 |
| RLVTASMVSPSF | 95 | 626 |
| DGQVSDHSYVQW | 101 | 627 |
| KISMNRLHANFT | 110 | 628 |
| WDLPTLRKTKQA | 114 | 629 |
| WVAAERR | 115 | 630 |
| MTHSAHNSQKTN | 117 | 631 |
| WGLKLPH | 117 | 632 |
| QGWFRHS | 121 | 633 |
| IPVGERK | 124 | 634 |
| CMNKIQRDC | 131 | 635 |
| KALGYSLVGGEW | 134 | 636 |
| APKVTNARPTQL | 147 | 637 |
| AWVEAQNASNPS | 160 | 638 |
| CSKHNHSRC | 295 | 639 |
| GKPDHRLVSLWR | 309 | 640 |
| CSHHKLKQC | 359 | 641 |
| GGYDLPWANWQN | 497 | 642 |

TABLE 4F

| | Count | | | |
|---|---|---|---|---|
| | | Excised | Nerve | SEQ |
| Peptide | Neuron | Nerve | Extract | ID NO: |
| GWQMFPPMQNTR | 40 | 10 | 50 | 643 |
| SSLYMNGWSRDQ | 26 | 23 | 49 | 644 |
| YAGQNGWMRSDL | 17 | 28 | 45 | 645 |
| VERLNWPKRMQS | 16 | 27 | 43 | 646 |
| WDVAWLERRALH | 20 | 23 | 43 | 647 |
| VRLPMLASMGHQ | 21 | 19 | 40 | 648 |
| DRGLTILTDDLR | 7 | 27 | 34 | 649 |
| GWNEATNSARHT | 6 | 26 | 32 | 650 |
| EFARFRVFGVAE | 8 | 22 | 30 | 651 |
| NRIGHGTATGTL | 6 | 22 | 28 | 652 |
| VTDMLGIPKLHA | 10 | 18 | 28 | 653 |
| DELVGGAPWYKS | 7 | 20 | 27 | 654 |
| GLLREPDELMFL | 9 | 18 | 27 | 655 |
| QNIVENFIDNER | 7 | 20 | 27 | 656 |
| SGEYNLIKPNLL | 7 | 20 | 27 | 657 |
| EESNAKLELLSR | 14 | 12 | 26 | 658 |
| GWANSITYRNLY | 9 | 17 | 26 | 659 |
| NYAVGDRITKNI | 7 | 18 | 25 | 660 |
| HGIVFSKRPFSN | 11 | 12 | 23 | 661 |
| FDPLSRNVPHKT | 6 | 16 | 22 | 662 |
| NAVHRPDLNLLA | 7 | 15 | 22 | 663 |
| GMRTGDDSPKYP | 7 | 14 | 21 | 664 |
| IPGQTPRMPHSK | 13 | 8 | 21 | 665 |
| KTTSMTALTMGL | 12 | 9 | 21 | 666 |
| VNYQTPWNKHWY | 11 | 10 | 21 | 667 |
| VESGHAYSVSSW | 6 | 14 | 20 | 668 |
| VRSDMVSGGLQK | 6 | 14 | 20 | 669 |
| YANGIEAKDSSR | 6 | 14 | 20 | 670 |
| YAWDTVASRNYK | 6 | 14 | 20 | 671 |
| LQTHSITYRHER | 12 | 7 | 19 | 672 |
| SLGSSFVQGGEL | 7 | 12 | 19 | 673 |
| WEKHTGPLSRYL | 13 | 6 | 19 | 674 |
| AFRFEFQVPDSH | 11 | 7 | 18 | 675 |
| GHAHSLTQNPLF | 8 | 10 | 18 | 676 |
| GLTIMSSRHSLQ | 6 | 12 | 18 | 677 |
| NGMNWLQVHQLS | 6 | 12 | 18 | 678 |
| WSGGMEDNSNRL | 9 | 9 | 18 | 679 |
| AIMSYNSPWIQG | 8 | 9 | 17 | 680 |
| DVMQMFHQVHFY | 7 | 10 | 17 | 681 |
| QANLYYWDPSDL | 9 | 8 | 17 | 682 |
| QRATNDILIRGW | 8 | 9 | 17 | 683 |
| SPMANVWFNRLS | 10 | 7 | 17 | 684 |
| YSANSNPKKSLM | 6 | 11 | 17 | 685 |
| ALNHNGPVKTGL | 6 | 10 | 16 | 686 |
| DASQYPLVSGLW | 6 | 10 | 16 | 687 |
| EPWQSRDKVIEE | 8 | 8 | 16 | 688 |
| GLWDGKGRIVEV | 7 | 9 | 16 | 689 |
| GTVSYMVKMDNN | 7 | 9 | 16 | 690 |
| SLQLWNSEKVTT | 6 | 10 | 16 | 691 |
| WVGPDIIAQMRI | 8 | 8 | 16 | 692 |
| EIIVMSGSNVTN | 6 | 9 | 15 | 693 |
| HSGAYTIQASYK | 6 | 9 | 15 | 694 |
| MLKMERPGRMAY | 6 | 9 | 15 | 695 |
| SDHTFKRVMFRW | 7 | 8 | 15 | 696 |
| EMVPMKFLMMAK | 6 | 8 | 14 | 697 |
| NTEYSRYTSIWK | 6 | 8 | 14 | 698 |
| TDNGSFLGKLTS | 7 | 7 | 14 | 699 |
| TEWMVRASSIGE | 8 | 6 | 14 | 700 |
| VPVPKKMSLDPT | 6 | 8 | 14 | 701 |
| WPLKRFEQLPLK | 8 | 6 | 14 | 702 |
| MLRHVDLTEPNA | 6 | 7 | 13 | 703 |
| QSTLGVHMLRVS | 7 | 6 | 13 | 704 |
| RTVITNEMLLLV | 6 | 7 | 13 | 705 |
| SEPIEKGSRTIV | 6 | 7 | 13 | 706 |
| TLNARDQLSING | 6 | 7 | 13 | 707 |
| GTALNLQKDINK | 6 | 6 | 12 | 708 |
| SGWHLVMSMSIG | 6 | 6 | 12 | 709 |
| TSAHHDRTLAGS | 6 | 6 | 12 | 710 |

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. Patent Application No. 63/121,825, filed on Dec. 4, 2020 and U.S. Patent Application No. 63/167,572, filed on Mar. 29, 2021, are incorporated herein by reference, in their entirety. Aspects of the embodiments can be modified, if necessary, to employ concepts of the various patents, applications and publications to provide yet further embodiments. Moreover, in certain instances, well-known methods, procedures, or other specific details have not been described to avoid unnecessarily obscuring aspects of the invention defined by the appended claims

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims 10 are not limited by the disclosure.

1. A nerve targeting peptide conjugate comprising:
(a) a peptide comprising:
(i) an amino acid sequence of any one of SEQ ID NOS:1-710;
(ii) an amino acid sequence of any one of SEQ ID NOS:1-66; or
(ii) an amino acid sequence of any one of SEQ ID NOS: 1, 2, 4-17, 23, 24, 29, 31, 34, 35, 37, 40, 41, 42, 44-47, 49, 50, 52-60, and 67-710;
(b) a cargo molecule.

2. The nerve targeting peptide conjugate of claim 1, wherein the peptide comprises the amino acid sequence of any one of SEQ ID NOS: 1-20 and 67-151.

3. The nerve targeting peptide conjugate of claim 1, wherein the peptide comprises the amino acid sequence of any one of SEQ ID NOS:21-40 and 152-326.

4. The nerve targeting peptide conjugate of claim 1, wherein the peptide comprises the amino acid sequence of any one of SEQ ID NOS:41-60 and 327-410.

5. The nerve targeting peptide conjugate of claim 1, wherein the peptide comprises the amino acid sequence of any one of SEQ ID NOS:61-66 and 411-435.

6. The nerve targeting peptide conjugate of claim 1, wherein the peptide comprises the amino acid sequence of any one of SEQ ID NOS:436-642.

7. The nerve targeting peptide conjugate of claim 1, wherein the peptide comprises the amino acid sequence of any one of SEQ ID NOS:643-710.

8. The nerve targeting peptide conjugate of any one of claims 1-7, wherein the cargo molecule comprises a drug, a fluorescent moiety, or a photosensitizing agent.

9. The nerve targeting peptide conjugate of any one of claims 1-8, wherein the cargo molecule is joined to the N-terminus or C-terminus of the peptide.

10. The nerve targeting peptide conjugate of any one of claims 1-9, wherein the cargo molecule is joined to the peptide via a linker.

11. The nerve targeting peptide of claim 10, wherein the linker is a straight or branched-chain carbon linker, heterocyclic carbon linker, amino acid linker, lipophilic residue, peptide linker, peptide nucleic acid linker, hydrazone linker, SPDB disulfide, sulfo-SPDB, maleimidomethyl cyclohexane-1-carboxylate (MCC), aminohexanoic acid linker, polyether linker, or polyethylene glycol linker.

12. The nerve targeting peptide conjugate of any one of claims 1-11, wherein the cargo molecule comprises a fluorescent moiety.

13. The nerve targeting peptide conjugate of claim 12, wherein the fluorescent moiety is a fluorescent protein, a fluorescent peptide, a fluorophore, or any combination thereof.

14. The nerve targeting peptide conjugate of claim 12 or 13, wherein the fluorescent moiety is selected from the group consisting of: a xanthene; a bimane; a coumarin; an aromatic amines; a benzofuran; a fluorescent cyanine; a carbazole; a dicyanomethylene pyrane; polymethine; oxabenzanthrane; pyrylium; carbostyl; perylene; acridone; quinacridone; rubrene; anthracene; coronene; phenanthrecene; pyrene; butadiene; stilbene; porphyrin; pthalocyanine; lanthanide metal chelate complexes; rare-earth metal chelate complexes; and derivatives thereof.

15. The nerve targeting peptide conjugate of claim 12 or 13, wherein the fluorescent moiety is selected from the group consisting of: 5-carboxyfluorescein; fluorescein-5-isothiocyanate; 6-carboxyfluorescein; tetramethylrhodamine-6-isothiocyanate; 5-carboxytetramethylrhodamine; 5-carboxy rhodol derivatives; tetramethyl and tetraethyl rhodamine; diphenyldimethyl and diphenyldiethyl rhodamine; dinaphthyl rhodamine; rhodamine 101 sulfonyl chloride; Cy3, Cy3B, Cy3.5, Cy5, Cy5.5, Cy 7, indocyanine green, IR800CW, cyan fluorescent protein (CFP), EGFP, 6-FAM, FAM, fluorescein, 5,6-dicarboxyfluorescein, 5-(and 6)-sulfofluorescein, sulfonefluorescein, succinyl fluorescein, 5-(and 6)-carboxy SNARF-1, carboxyfluorescein sulfonate, carboxyfluorescein zwitterion, carboxyfluorescein quaternary ammonium, carboxyfluorescein phosphonate, carboxyfluorescein GABA, carboxyfluorescein-cys-Cy5,5′(6′)-carboxyfluorescein, fluorescein glutathione, or any combination thereof.

16. The nerve targeting peptide conjugate of any one of claims 1-11, wherein the cargo molecule comprises a photosensitizing agent.

17. The nerve targeting peptide conjugate of claim 16, wherein the photosensitizing agent is selected from the group consisting of: a porphyrin, chlorin, and dye.

18. The nerve targeting peptide conjugate of claim 16, wherein the photosensitizing agent is selected from the group consisting of: porphyrin, protoporfin IX, purlytin, verteporfin, HPPH, temoporfin, methylene blue, photofrin, protofrin, hematoporphyrin, Talaporfin, benzopophyrin derivative monoacid, 5-aminileuvolinic acid, Lutetium texaphyrin, metallophthalocyanine, metallo-naphthocyaninesulfobenzo-porphyrazine, metallo-naphthalocyanines, zinc tetrasulfophthalocyanine, bacteriochlorins, metallochlorins, chlorine derivative, Tetra(m-hydroxyphenyl)chlorin (mTHPC), pheophorbide, dibromofluorescein (DBF), IR 700DX, naphthalocyanine, and porphyrin derivatives.

19. The nerve targeting peptide conjugate of any one of claims 1-11, wherein the cargo molecule comprises a drug.

20. The nerve targeting peptide conjugate of claim 19, wherein the drug is selected from the group consisting of: an antihistamine, a GABA receptor modulator, a neurotransmitter reuptake inhibitor, a local anesthetic, an anticholinergic, a sodium channel blocker, a calcium channel blocker, a thyrotropin-releasing hormone, a γ-secretase inhibitor, an AMPA receptor agonist or antagonist, an NMDA receptor agonist or antagonist, an mGlu receptor agonist or antagonist, a growth factor, an antiemetic agent, a corticosteroid; a nonsteroidal anti-inflammatory drug (NSAID); an anti-epileptic agent; a neurotropic agent; a cytotoxic agent; an antioxidant, an iron chelator, a mitochondrial modulator, a sirtuin modulator, a nitric oxide (NO) and/or nitric oxide synthase (NOS) modulator, a potassium channel agonist or antagonist, a purigenic receptor agonist or antagonist, or any combination thereof.

21. The nerve targeting peptide conjugate of claim 19, wherein the drug is selected from the group consisting of: benzocaine; carticaine; cinchocaine; cyclomethycaine; lidocaine; prilocaine; propxycaine; proparacaine; tetracaine; tocainide; and trimecaine; methotrexate; cyclophosphamide; thalidomide; paclitaxel; pemetrexed; pentostatin; pipobroman; pixantrone; plicamycin; platonin; procarbazine; raltitrexed; rebeccamycin; rubitecan; SN-38; salinosporamide A; satraplatin; streptozotocin; swainsonine; tariquidar; taxane; tegafur-uracil; temozolomide; testolactone; thioTEPA; tioguanine; topotecan; trabectedin; tretinoin; triplatin tetranitrate; tris(2-chloroethyl)amine; troxacitabine; uracil mustard; valrubicin; vinblastine; vincristine; vinorelbine; vorinostat; zosuquidar; carbamazepine; oxcarbazepine; phenytein; valproic acid; sodium valproate; cinnarizine; flunarizine; nimodipine; brain-derived neurotrophic factor (BDNF); ciliary neurotrophic factor (CNTF); glial cell-line derived neurotrophic factor (GDNF); neurotrophin-3; neurotrophin-4; fibroblast growth factor (FGF) receptor; insulin-like growth factor (IGF); prednisone; prednisolone; dexamethasone; gabapentin; or any combination thereof.

22. A pharmaceutical composition comprising the nerve targeting peptide conjugate of any one of claims 1-21 and a pharmaceutically acceptable carrier.

23. A method of delivering a cargo molecule to a neuron or nerve comprising contacting the neuron or nerve with the nerve targeting peptide conjugate of any one of claims 1-21 or the pharmaceutical composition of claim 22.

24. A method of identifying a neuron or nerve comprising contacting the neuron or nerve with the nerve targeting peptide conjugate of any one of claims 12-15.

25. A method of delivering a drug to a neuron or nerve comprising contacting the neuron or nerve with the nerve targeting peptide conjugate of any one of claims 19-21.

26. A method of delivering a photosensitizing agent to a neuron or nerve comprising contacting the neuron or nerve with the nerve targeting peptide conjugate of any one of claims 16-18.

27. The method of claim 26, further comprising exposing the neuron or nerve to a light source that activates the photosensitizing agent, wherein the neuron or nerve is ablated by the activated photosensitizing agent.

28. The method of any one of claims 23-27, wherein the neuron or nerve is a human neuron or human nerve.

29. The method of any one of claims 23-27, wherein the method is performed in a subject.

30. The method of claim 29, wherein the nerve targeting peptide conjugate is administered intravenously or topically to the subject.

31. The method of claim 29, wherein the nerve targeting peptide conjugate is administered orally to the subject.

32. The method of any one of claims 23-31, wherein the nerve targeting peptide conjugate is administered prior to a surgical procedure on the subject.

33. The method of claim 32, wherein the surgical procedure is performed on the head, neck, spine, heart, or prostate.

34. The method of claim 32 or 33, wherein the surgical procedure is an open surgical procedure, a laparoscopic surgical procedure, a microscopic procedure, or an endoscopic procedure.


SEQUENCE LISTING (721 sequences)

SEQ ID NO: 1 - protein, 7 aa, Artificial Sequence
  R3 Human MPZ-binding peptide
ALPLSAS

SEQ ID NO: 2 - protein, 7 aa, Artificial Sequence
  R3 Human MPZ-binding peptide
TSPWELR

SEQ ID NO: 3 - protein, 7 aa, Artificial Sequence
  R3 Human MPZ-binding peptide
SHLASRP

SEQ ID NO: 4 - protein, 7 aa, Artificial Sequence
  R3 Human MPZ-binding peptide
DPITRLK

SEQ ID NO: 5 - protein, 7 aa, Artificial Sequence
  R3 Human MPZ-binding peptide
VLKNNPT

SEQ ID NO: 6 - protein, 7 aa, Artificial Sequence
  R3 Human MPZ-binding peptide
IKPPYTH

SEQ ID NO: 7 - protein, 7 aa, Artificial Sequence
  R3 Human MPZ-binding peptide
ETTRNYG

SEQ ID NO: 8 - protein, 7 aa, Artificial Sequence
  R3 Human MPZ-binding peptide
SHSPGNK

SEQ ID NO: 9 - protein, 7 aa, Artificial Sequence
  R3 Human MPZ-binding peptide
WPSISRP

SEQ ID NO: 10 - protein, 7 aa, Artificial Sequence
  R3 Human MPZ-binding peptide
LSSPLSL

SEQ ID NO: 11 - protein, 7 aa, Artificial Sequence
  R3 Human MPZ-binding peptide
VLTKPMP

SEQ ID NO: 12 - protein, 7 aa, Artificial Sequence
  R3 Human MPZ-binding peptide
TMASPAK

SEQ ID NO: 13 - protein, 7 aa, Artificial Sequence
  R3 Human MPZ-binding peptide
KMAGHTV

SEQ ID NO: 14 - protein, 7 aa, Artificial Sequence
  R3 Human MPZ-binding peptide
SAHAKHH

SEQ ID NO: 15 - protein, 7 aa, Artificial Sequence
  R3 Human MPZ-binding peptide
DRNSVFW

SEQ ID NO: 16 - protein, 7 aa, Artificial Sequence
  R3 Human MPZ-binding peptide
GPLRMAT

SEQ ID NO: 17 - protein, 7 aa, Artificial Sequence
  R3 Human MPZ-binding peptide
TESLFPH

SEQ ID NO: 18 - protein, 7 aa, Artificial Sequence
  R3 Human MPZ-binding peptide
RVPHSRT

SEQ ID NO: 19 - protein, 7 aa, Artificial Sequence
  R3 Human MPZ-binding peptide
GLTPKLF

SEQ ID NO: 20 - protein, 12 aa, Artificial Sequence
  R3 Human MPZ-binding peptide
GDERIVRTLSHN

SEQ ID NO: 21 - protein, 7 aa, Artificial Sequence
  R3 Myelin Basic Protein-binding peptide
NDTKTPS

SEQ ID NO: 22 - protein, 7 aa, Artificial Sequence
  R3 Myelin Basic Protein-binding peptide
HPSTWHK

SEQ ID NO: 23 - protein, 7 aa, Artificial Sequence
  R3 Myelin Basic Protein-binding peptide
EHGYYKV

SEQ ID NO: 24 - protein, 7 aa, Artificial Sequence
  R3 Myelin Basic Protein-binding peptide
LPMRILT

SEQ ID NO: 25 - protein, 7 aa, Artificial Sequence
  R3 Myelin Basic Protein-binding peptide
SVLRMLN

SEQ ID NO: 26 - protein, 7 aa, Artificial Sequence
  R3 Myelin Basic Protein-binding peptide
AITSRNA

SEQ ID NO: 27 - protein, 7 aa, Artificial Sequence
  R3 Myelin Basic Protein-binding peptide
SKSDAWR

SEQ ID NO: 28 - protein, 7 aa, Artificial Sequence
  R3 Myelin Basic Protein-binding peptide
ILLPSLN

SEQ ID NO: 29 - protein, 7 aa, Artificial Sequence
  R3 Myelin Basic Protein-binding peptide
WTMKPNY

SEQ ID NO: 30 - protein, 10 aa, Artificial Sequence
  R3 Myelin Basic Protein-binding peptide
AGQLMRCANC

SEQ ID NO: 31 - protein, 7 aa, Artificial Sequence
  R3 Myelin Basic Protein-binding peptide
AWSTSSV

SEQ ID NO: 32 - protein, 7 aa, Artificial Sequence
  R3 Myelin Basic Protein-binding peptide
LRPISHE

SEQ ID NO: 33 - protein, 7 aa, Artificial Sequence
  R3 Myelin Basic Protein-binding peptide
MQTQGRV

SEQ ID NO: 34 - protein, 7 aa, Artificial Sequence
  R3 Myelin Basic Protein-binding peptide
SPDRTSL

SEQ ID NO: 35 - protein, 7 aa, Artificial Sequence
  R3 Myelin Basic Protein-binding peptide
DPWVMLR

SEQ ID NO: 36 - protein, 7 aa, Artificial Sequence
  R3 Myelin Basic Protein-binding peptide
QIGVLPS

SEQ ID NO: 37 - protein, 7 aa, Artificial Sequence
  R3 Myelin Basic Protein-binding peptide
QNTGLKW

SEQ ID NO: 38 - protein, 7 aa, Artificial Sequence
  R3 Myelin Basic Protein-binding peptide
AGFPPNT

SEQ ID NO: 39 - protein, 7 aa, Artificial Sequence
  R3 Myelin Basic Protein-binding peptide
DGKNTSN

SEQ ID NO: 40 - protein, 7 aa, Artificial Sequence
  R3 Myelin Basic Protein-binding peptide
HRLPWHH

SEQ ID NO: 41 - protein, 7 aa, Artificial Sequence
  R3 Myelin PLP-binding peptide
ATLKPYR

SEQ ID NO: 42 - protein, 7 aa, Artificial Sequence
  R3 Myelin PLP-binding peptide
GLIRHYT

SEQ ID NO: 43 - protein, 7 aa, Artificial Sequence
  R3 Myelin PLP-binding peptide
MTPHTSC

SEQ ID NO: 44 - protein, 7 aa, Artificial Sequence
  R3 Myelin PLP-binding peptide
TNPWKPH

SEQ ID NO: 45 - protein, 7 aa, Artificial Sequence
  R3 Myelin PLP-binding peptide
HQGSRTY

SEQ ID NO: 46 - protein, 7 aa, Artificial Sequence
  R3 Myelin PLP-binding peptide
RFELPAP

SEQ ID NO: 47 - protein, 7 aa, Artificial Sequence
  R3 Myelin PLP-binding peptide
YTPMGTG

SEQ ID NO: 48 - protein, 7 aa, Artificial Sequence
  R3 Myelin PLP-binding peptide
GPFLFTV

SEQ ID NO: 49 - protein, 7 aa, Artificial Sequence
  R3 Myelin PLP-binding peptide
LALYARM

SEQ ID NO: 50 - protein, 7 aa, Artificial Sequence
  R3 Myelin PLP-binding peptide
LRFQIPP

SEQ ID NO: 51 - protein, 7 aa, Artificial Sequence
  R3 Myelin PLP-binding peptide
FPVVTRN

SEQ ID NO: 52 - protein, 7 aa, Artificial Sequence
  R3 Myelin PLP-binding peptide
RHNVEFS

SEQ ID NO: 53 - protein, 7 aa, Artificial Sequence
  R3 Myelin PLP-binding peptide
LTHHFAP

SEQ ID NO: 54 - protein, 7 aa, Artificial Sequence
  R3 Myelin PLP-binding peptide
NPSHHPR

SEQ ID NO: 55 - protein, 7 aa, Artificial Sequence
  R3 Myelin PLP-binding peptide
YPKLWTY

SEQ ID NO: 56 - protein, 7 aa, Artificial Sequence
  R3 Myelin PLP-binding peptide
FKMPIPT

SEQ ID NO: 57 - protein, 7 aa, Artificial Sequence
  R3 Myelin PLP-binding peptide
NPNWRTI

SEQ ID NO: 58 - protein, 7 aa, Artificial Sequence
  R3 Myelin PLP-binding peptide
SMWSQFR

SEQ ID NO: 59 - protein, 7 aa, Artificial Sequence
  R3 Myelin PLP-binding peptide
DTSRTME

SEQ ID NO: 60 - protein, 7 aa, Artificial Sequence
  R3 Myelin PLP-binding peptide
EKPSWTR

SEQ ID NO: 61 - protein, 10 aa, Artificial Sequence
  R3 Nidogen-2-binding peptide
ACGEGEADVC

SEQ ID NO: 62 - protein, 10 aa, Artificial Sequence
  R3 Nidogen-2-binding peptide
ACPENKSKHC

SEQ ID NO: 63 - protein, 10 aa, Artificial Sequence
  R3 Nidogen-2-binding peptide
ACEPRSLANC

SEQ ID NO: 64 - protein, 10 aa, Artificial Sequence
  R3 Nidogen-2-binding peptide
ACTVHKWDNC

SEQ ID NO: 65 - protein, 7 aa, Artificial Sequence
  R3 Nidogen-2-binding peptide
GYWRNAL

SEQ ID NO: 66 - protein, 8 aa, Artificial Sequence
  R3 Nidogen-2-binding peptide
KAIHPMRG

SEQ ID NO: 67 - protein, 9 aa, Artificial Sequence
  Human MPZ-binding peptide
CLKQQTGDC

SEQ ID NO: 68 - protein, 12 aa, Artificial Sequence
  Human MPZ-binding peptide
EGHVWSEYTWGT

SEQ ID NO: 69 - protein, 7 aa, Artificial Sequence
  Human MPZ-binding peptide
FMSDVDH

SEQ ID NO: 70 - protein, 7 aa, Artificial Sequence
  Human MPZ-binding peptide
ISSVTVV

SEQ ID NO: 71 - protein, 7 aa, Artificial Sequence
  Human MPZ-binding peptide
KQPAMFN

SEQ ID NO: 72 - protein, 7 aa, Artificial Sequence
  Human MPZ-binding peptide
LISPTAR

SEQ ID NO: 73 - protein, 7 aa, Artificial Sequence
  Human MPZ-binding peptide
LVLQTTP

SEQ ID NO: 74 - protein, 7 aa, Artificial Sequence
  Human MPZ-binding peptide
NPTSEGA

SEQ ID NO: 75 - protein, 7 aa, Artificial Sequence
  Human MPZ-binding peptide
THSTDLS

SEQ ID NO: 76 - protein, 12 aa, Artificial Sequence
  Human MPZ-binding peptide
TTNVTEIERESY

SEQ ID NO: 77 - protein, 7 aa, Artificial Sequence
  Human MPZ-binding peptide
YAPWRVF

SEQ ID NO: 78 - protein, 7 aa, Artificial Sequence
  Human MPZ-binding peptide
YGQSITE

SEQ ID NO: 79 - protein, 9 aa, Artificial Sequence
  Human MPZ-binding peptide
CDIKDANSC

SEQ ID NO: 80 - protein, 9 aa, Artificial Sequence
  Human MPZ-binding peptide
CIKGEDNQC

SEQ ID NO: 81 - protein, 7 aa, Artificial Sequence
  Human MPZ-binding peptide
HNQLLYT

SEQ ID NO: 82 - protein, 7 aa, Artificial Sequence
  Human MPZ-binding peptide
IEQVGWR

SEQ ID NO: 83 - protein, 7 aa, Artificial Sequence
  Human MPZ-binding peptide
KTPILAS

SEQ ID NO: 84 - protein, 7 aa, Artificial Sequence
  Human MPZ-binding peptide
LWAKRDA

SEQ ID NO: 85 - protein, 7 aa, Artificial Sequence
  Human MPZ-binding peptide
NPISPRN

SEQ ID NO: 86 - protein, 7 aa, Artificial Sequence
  Human MPZ-binding peptide
NTASMTT

SEQ ID NO: 87 - protein, 7 aa, Artificial Sequence
  Human MPZ-binding peptide
SAHSVVE

SEQ ID NO: 88 - protein, 7 aa, Artificial Sequence
  Human MPZ-binding peptide
YSSLTSN

SEQ ID NO: 89 - protein, 7 aa, Artificial Sequence
  Human MPZ-binding peptide
AWTMRAS

SEQ ID NO: 90 - protein, 9 aa, Artificial Sequence
  Human MPZ-binding peptide
CERIGPREC

SEQ ID NO: 91 - protein, 9 aa, Artificial Sequence
  Human MPZ-binding peptide
CQKWFTFAC

SEQ ID NO: 92 - protein, 9 aa, Artificial Sequence
  Human MPZ-binding peptide
CSNALNKAC

SEQ ID NO: 93 - protein, 7 aa, Artificial Sequence
  Human MPZ-binding peptide
FSPRDVS

SEQ ID NO: 94 - protein, 7 aa, Artificial Sequence
  Human MPZ-binding peptide
FSTTGRG

SEQ ID NO: 95 - protein, 7 aa, Artificial Sequence
  Human MPZ-binding peptide
HHPLFID

SEQ ID NO: 96 - protein, 7 aa, Artificial Sequence
  Human MPZ-binding peptide
LVNPFPE

SEQ ID NO: 97 - protein, 7 aa, Artificial Sequence
  Human MPZ-binding peptide
YEGERGA

SEQ ID NO: 98 - protein, 9 aa, Artificial Sequence
  Human MPZ-binding peptide
CEDRQRSMC

SEQ ID NO: 99 - protein, 9 aa, Artificial Sequence
  Human MPZ-binding peptide
CGEKIMSLC

SEQ ID NO: 100 - protein, 7 aa, Artificial Sequence
  Human MPZ-binding peptide
DRYLLNQ

SEQ ID NO: 101 - protein, 7 aa, Artificial Sequence
  Human MPZ-binding peptide
FPSYTIS

SEQ ID NO: 102 - protein, 7 aa, Artificial Sequence
  Human MPZ-binding peptide
GGSKVWG

SEQ ID NO: 103 - protein, 7 aa, Artificial Sequence
  Human MPZ-binding peptide
GLWRHTM

SEQ ID NO: 104 - protein, 7 aa, Artificial Sequence
  Human MPZ-binding peptide
KRNLWDI

SEQ ID NO: 105 - protein, 7 aa, Artificial Sequence
  Human MPZ-binding peptide
SPNTNSH

SEQ ID NO: 106 - protein, 7 aa, Artificial Sequence
  Human MPZ-binding peptide
TLLHYSA

SEQ ID NO: 107 - protein, 7 aa, Artificial Sequence
  Human MPZ-binding peptide
VPLAYLR

SEQ ID NO: 108 - protein, 7 aa, Artificial Sequence
  Human MPZ-binding peptide
VVPLKWI

SEQ ID NO: 109 - protein, 9 aa, Artificial Sequence
  Human MPZ-binding peptide
CRVGVTSAC

SEQ ID NO: 110 - protein, 7 aa, Artificial Sequence
  Human MPZ-binding peptide
FALPAGL

SEQ ID NO: 111 - protein, 7 aa, Artificial Sequence
  Human MPZ-binding peptide
FRVTQTA

SEQ ID NO: 112 - protein, 7 aa, Artificial Sequence
  Human MPZ-binding peptide
HPLMVPS

SEQ ID NO: 113 - protein, 7 aa, Artificial Sequence
  Human MPZ-binding peptide
LPNANNL

SEQ ID NO: 114 - protein, 7 aa, Artificial Sequence
  Human MPZ-binding peptide
MIHTRQT

SEQ ID NO: 115 - protein, 7 aa, Artificial Sequence
  Human MPZ-binding peptide
SETESYM

SEQ ID NO: 116 - protein, 7 aa, Artificial Sequence
  Human MPZ-binding peptide
SNALSYF

SEQ ID NO: 117 - protein, 7 aa, Artificial Sequence
  Human MPZ-binding peptide
SYLPPFI

SEQ ID NO: 118 - protein, 7 aa, Artificial Sequence
  Human MPZ-binding peptide
VHTPYRS

SEQ ID NO: 119 - protein, 7 aa, Artificial Sequence
  Human MPZ-binding peptide
WPRPIQI

SEQ ID NO: 120 - protein, 7 aa, Artificial Sequence
  Human MPZ-binding peptide
WTSVATA

SEQ ID NO: 121 - protein, 7 aa, Artificial Sequence
  Human MPZ-binding peptide
ALPISYL

SEQ ID NO: 122 - protein, 7 aa, Artificial Sequence
  Human MPZ-binding peptide
FEHMAVT

SEQ ID NO: 123 - protein, 7 aa, Artificial Sequence
  Human MPZ-binding peptide
NASVPPK

SEQ ID NO: 124 - protein, 7 aa, Artificial Sequence
  Human MPZ-binding peptide
SGHGAFR

SEQ ID NO: 125 - protein, 7 aa, Artificial Sequence
  Human MPZ-binding peptide
TTLVVTA

SEQ ID NO: 126 - protein, 9 aa, Artificial Sequence
  Human MPZ-binding peptide
CPDAARNSC

SEQ ID NO: 127 - protein, 9 aa, Artificial Sequence
  Human MPZ-binding peptide
CYSSVADMC

SEQ ID NO: 128 - protein, 7 aa, Artificial Sequence
  Human MPZ-binding peptide
LSPPRIM

SEQ ID NO: 129 - protein, 7 aa, Artificial Sequence
  Human MPZ-binding peptide
WMTNLDP

SEQ ID NO: 130 - protein, 7 aa, Artificial Sequence
  Human MPZ-binding peptide
KLPTLSV

SEQ ID NO: 131 - protein, 7 aa, Artificial Sequence
  Human MPZ-binding peptide
AMSNLSY

SEQ ID NO: 132 - protein, 7 aa, Artificial Sequence
  Human MPZ-binding peptide
HPFPRFD

SEQ ID NO: 133 - protein, 7 aa, Artificial Sequence
  Human MPZ-binding peptide
LPAESHW

SEQ ID NO: 134 - protein, 7 aa, Artificial Sequence
  Human MPZ-binding peptide
STHEWRT

SEQ ID NO: 135 - protein, 7 aa, Artificial Sequence
  Human MPZ-binding peptide
TTWPNTA

SEQ ID NO: 136 - protein, 7 aa, Artificial Sequence
  Human MPZ-binding peptide
YMKHSPG

SEQ ID NO: 137 - protein, 12 aa, Artificial Sequence
  Human MPZ-binding peptide
AYLEDWRSMSTR

SEQ ID NO: 138 - protein, 7 aa, Artificial Sequence
  Human MPZ-binding peptide
GPVVSGM

SEQ ID NO: 139 - protein, 7 aa, Artificial Sequence
  Human MPZ-binding peptide
HLDSRRH

SEQ ID NO: 140 - protein, 7 aa, Artificial Sequence
  Human MPZ-binding peptide
LVLSNPR

SEQ ID NO: 141 - protein, 7 aa, Artificial Sequence
  Human MPZ-binding peptide
NMSHSNR

SEQ ID NO: 142 - protein, 7 aa, Artificial Sequence
  Human MPZ-binding peptide
SVAYVPV

SEQ ID NO: 143 - protein, 7 aa, Artificial Sequence
  Human MPZ-binding peptide
YPQARTS

SEQ ID NO: 144 - protein, 7 aa, Artificial Sequence
  Human MPZ-binding peptide
MYHDSVS

SEQ ID NO: 145 - protein, 12 aa, Artificial Sequence
  Human MPZ-binding peptide
ADNVLRRALENI

SEQ ID NO: 146 - protein, 7 aa, Artificial Sequence
  Human MPZ-binding peptide
QIAHLEY

SEQ ID NO: 147 - protein, 12 aa, Artificial Sequence
  Human MPZ-binding peptide
GMVSPHHSVYRH

SEQ ID NO: 148 - protein, 7 aa, Artificial Sequence
  Human MPZ-binding peptide
GSWSSGF

SEQ ID NO: 149 - protein, 12 aa, Artificial Sequence
  Human MPZ-binding peptide
NGAYSLAIRYTS

SEQ ID NO: 150 - protein, 12 aa, Artificial Sequence
  Human MPZ-binding peptide
YTPNWHFRWMPA

SEQ ID NO: 151 - protein, 12 aa, Artificial Sequence
  Human MPZ-binding peptide
GEWRARIDQDVS

SEQ ID NO: 152 - protein, 7 aa, Artificial Sequence
  R3 Myelin Basic Protein-binding peptide
AGSLLSL

SEQ ID NO: 153 - protein, 7 aa, Artificial Sequence
  R3 Myelin Basic Protein-binding peptide
AIPPRKL

SEQ ID NO: 154 - protein, 7 aa, Artificial Sequence
  R3 Myelin Basic Protein-binding peptide
AISWKGF

SEQ ID NO: 155 - protein, 7 aa, Artificial Sequence
  R3 Myelin Basic Protein-binding peptide
AKFTMWV

SEQ ID NO: 156 - protein, 7 aa, Artificial Sequence
  R3 Myelin Basic Protein-binding peptide
ALRDTRV

SEQ ID NO: 157 - protein, 7 aa, Artificial Sequence
  R3 Myelin Basic Protein-binding peptide
ALYNQMT

SEQ ID NO: 158 - protein, 7 aa, Artificial Sequence
  R3 Myelin Basic Protein-binding peptide
AQFLTIY

SEQ ID NO: 159 - protein, 7 aa, Artificial Sequence
  R3 Myelin Basic Protein-binding peptide
AQLGAFR

SEQ ID NO: 160 - protein, 7 aa, Artificial Sequence
  R3 Myelin Basic Protein-binding peptide
ARVPNPL

SEQ ID NO: 161 - protein, 9 aa, Artificial Sequence
  R3 Myelin Basic Protein-binding peptide
CTMTNQYDC

SEQ ID NO: 162 - protein, 7 aa, Artificial Sequence
  R3 Myelin Basic Protein-binding peptide
DSQEKSV

SEQ ID NO: 163 - protein, 7 aa, Artificial Sequence
  R3 Myelin Basic Protein-binding peptide
DVRIWTL

SEQ ID NO: 164 - protein, 7 aa, Artificial Sequence
  R3 Myelin Basic Protein-binding peptide
ELKSLRF

SEQ ID NO: 165 - protein, 7 aa, Artificial Sequence
  R3 Myelin Basic Protein-binding peptide
ESKSPRE

SEQ ID NO: 166 - protein, 7 aa, Artificial Sequence
  R3 Myelin Basic Protein-binding peptide
ETHYKIA

SEQ ID NO: 167 - protein, 7 aa, Artificial Sequence
  R3 Myelin Basic Protein-binding peptide
FDLLARK

SEQ ID NO: 168 - protein, 7 aa, Artificial Sequence
  R3 Myelin Basic Protein-binding peptide
FQLSYET

SEQ ID NO: 169 - protein, 7 aa, Artificial Sequence
  R3 Myelin Basic Protein-binding peptide
GEPALVT

SEQ ID NO: 170 - protein, 7 aa, Artificial Sequence
  R3 Myelin Basic Protein-binding peptide
GPRVTPH

SEQ ID NO: 171 - protein, 7 aa, Artificial Sequence
  R3 Myelin Basic Protein-binding peptide
GQWWKML

SEQ ID NO: 172 - protein, 7 aa, Artificial Sequence
  R3 Myelin Basic Protein-binding peptide
GRHTDVV

SEQ ID NO: 173 - protein, 7 aa, Artificial Sequence
  R3 Myelin Basic Protein-binding peptide
GSPYGWG

SEQ ID NO: 174 - protein, 7 aa, Artificial Sequence
  R3 Myelin Basic Protein-binding peptide
GVAEVGL

SEQ ID NO: 175 - protein, 7 aa, Artificial Sequence
  R3 Myelin Basic Protein-binding peptide
HAHWARL

SEQ ID NO: 176 - protein, 7 aa, Artificial Sequence
  R3 Myelin Basic Protein-binding peptide
HFTFFPI

SEQ ID NO: 177 - protein, 7 aa, Artificial Sequence
  R3 Myelin Basic Protein-binding peptide
HPWSHPN

SEQ ID NO: 178 - protein, 7 aa, Artificial Sequence
  R3 Myelin Basic Protein-binding peptide
HQMRTML

SEQ ID NO: 179 - protein, 7 aa, Artificial Sequence
  R3 Myelin Basic Protein-binding peptide
HQPLFPR

SEQ ID NO: 180 - protein, 7 aa, Artificial Sequence
  R3 Myelin Basic Protein-binding peptide
HRTHLHQ

SEQ ID NO: 181 - protein, 7 aa, Artificial Sequence
  R3 Myelin Basic Protein-binding peptide
HSGPLLP

SEQ ID NO: 182 - protein, 7 aa, Artificial Sequence
  R3 Myelin Basic Protein-binding peptide
HSSATMS

SEQ ID NO: 183 - protein, 7 aa, Artificial Sequence
  R3 Myelin Basic Protein-binding peptide
IGTRWHQ

SEQ ID NO: 184 - protein, 7 aa, Artificial Sequence
  R3 Myelin Basic Protein-binding peptide
INFPFAV

SEQ ID NO: 185 - protein, 7 aa, Artificial Sequence
  R3 Myelin Basic Protein-binding peptide
IPIPYRT

SEQ ID NO: 186 - protein, 7 aa, Artificial Sequence
  R3 Myelin Basic Protein-binding peptide
IPYSQMP

SEQ ID NO: 187 - protein, 7 aa, Artificial Sequence
  R3 Myelin Basic Protein-binding peptide
KIITTKS

SEQ ID NO: 188 - protein, 7 aa, Artificial Sequence
  R3 Myelin Basic Protein-binding peptide
KMSLAPP

SEQ ID NO: 189 - protein, 7 aa, Artificial Sequence
  R3 Myelin Basic Protein-binding peptide
KVHHLAR

SEQ ID NO: 190 - protein, 7 aa, Artificial Sequence
  R3 Myelin Basic Protein-binding peptide
LELHQNV

SEQ ID NO: 191 - protein, 7 aa, Artificial Sequence
  R3 Myelin Basic Protein-binding peptide
LITHPQL

SEQ ID NO: 192 - protein, 7 aa, Artificial Sequence
  R3 Myelin Basic Protein-binding peptide
LMLPQLE

SEQ ID NO: 193 - protein, 7 aa, Artificial Sequence
  R3 Myelin Basic Protein-binding peptide
LVPPVNH

SEQ ID NO: 194 - protein, 7 aa, Artificial Sequence
  R3 Myelin Basic Protein-binding peptide
MNSPLAQ

SEQ ID NO: 195 - protein, 7 aa, Artificial Sequence
  R3 Myelin Basic Protein-binding peptide
MQRLGAD

SEQ ID NO: 196 - protein, 7 aa, Artificial Sequence
  R3 Myelin Basic Protein-binding peptide
NGPNSLQ

SEQ ID NO: 197 - protein, 7 aa, Artificial Sequence
  R3 Myelin Basic Protein-binding peptide
NGVSSSL

SEQ ID NO: 198 - protein, 7 aa, Artificial Sequence
  R3 Myelin Basic Protein-binding peptide
NLTYLRF

SEQ ID NO: 199 - protein, 7 aa, Artificial Sequence
  R3 Myelin Basic Protein-binding peptide
QGAYVRP

SEQ ID NO: 200 - protein, 7 aa, Artificial Sequence
  R3 Myelin Basic Protein-binding peptide
QKALPQT

SEQ ID NO: 201 - protein, 7 aa, Artificial Sequence
  R3 Myelin Basic Protein-binding peptide
QTSIMSG

SEQ ID NO: 202 - protein, 7 aa, Artificial Sequence
  R3 Myelin Basic Protein-binding peptide
QYWDILG

SEQ ID NO: 203 - protein, 7 aa, Artificial Sequence
  R3 Myelin Basic Protein-binding peptide
RGHPSQL

SEQ ID NO: 204 - protein, 7 aa, Artificial Sequence
  R3 Myelin Basic Protein-binding peptide
RSRKHRP

SEQ ID NO: 205 - protein, 7 aa, Artificial Sequence
  R3 Myelin Basic Protein-binding peptide
RTTDWWT

SEQ ID NO: 206 - protein, 7 aa, Artificial Sequence
  R3 Myelin Basic Protein-binding peptide
RVIPSTT

SEQ ID NO: 207 - protein, 7 aa, Artificial Sequence
  R3 Myelin Basic Protein-binding peptide
SAVLMPP

SEQ ID NO: 208 - protein, 7 aa, Artificial Sequence
  R3 Myelin Basic Protein-binding peptide
SEGLTLL

SEQ ID NO: 209 - protein, 7 aa, Artificial Sequence
  R3 Myelin Basic Protein-binding peptide
SGPFPLV

SEQ ID NO: 210 - protein, 7 aa, Artificial Sequence
  R3 Myelin Basic Protein-binding peptide
SIQTSAM

SEQ ID NO: 211 - protein, 7 aa, Artificial Sequence
  R3 Myelin Basic Protein-binding peptide
SKLAGFP

SEQ ID NO: 212 - protein, 7 aa, Artificial Sequence
  R3 Myelin Basic Protein-binding peptide
SKPHASH

SEQ ID NO: 213 - protein, 7 aa, Artificial Sequence
  R3 Myelin Basic Protein-binding peptide
SLDHLRL

SEQ ID NO: 214 - protein, 7 aa, Artificial Sequence
  R3 Myelin Basic Protein-binding peptide
SLKTTNP

SEQ ID NO: 215 - protein, 7 aa, Artificial Sequence
  R3 Myelin Basic Protein-binding peptide
SLWQTLR

SEQ ID NO: 216 - protein, 7 aa, Artificial Sequence
  R3 Myelin Basic Protein-binding peptide
SLWTHRT

SEQ ID NO: 217 - protein, 7 aa, Artificial Sequence
  R3 Myelin Basic Protein-binding peptide
SNAHTLP

SEQ ID NO: 218 - protein, 7 aa, Artificial Sequence
  R3 Myelin Basic Protein-binding peptide
SPKPDIP

SEQ ID NO: 219 - protein, 7 aa, Artificial Sequence
  R3 Myelin Basic Protein-binding peptide
SRHLPPL

SEQ ID NO: 220 - protein, 7 aa, Artificial Sequence
  R3 Myelin Basic Protein-binding peptide
SSFYVAY

SEQ ID NO: 221 - protein, 7 aa, Artificial Sequence
  R3 Myelin Basic Protein-binding peptide
SSMRYNS

SEQ ID NO: 222 - protein, 7 aa, Artificial Sequence
  R3 Myelin Basic Protein-binding peptide
SVGLPTK

SEQ ID NO: 223 - protein, 7 aa, Artificial Sequence
  R3 Myelin Basic Protein-binding peptide
SYRSMAP

SEQ ID NO: 224 - protein, 7 aa, Artificial Sequence
  R3 Myelin Basic Protein-binding peptide
SYVTVFR

SEQ ID NO: 225 - protein, 7 aa, Artificial Sequence
  R3 Myelin Basic Protein-binding peptide
TGPSSAV

SEQ ID NO: 226 - protein, 7 aa, Artificial Sequence
  R3 Myelin Basic Protein-binding peptide
TGSVSKQ

SEQ ID NO: 227 - protein, 7 aa, Artificial Sequence
  R3 Myelin Basic Protein-binding peptide
THKHMMT

SEQ ID NO: 228 - protein, 7 aa, Artificial Sequence
  R3 Myelin Basic Protein-binding peptide
TIRVAHT

SEQ ID NO: 229 - protein, 7 aa, Artificial Sequence
  R3 Myelin Basic Protein-binding peptide
TPGRATL

SEQ ID NO: 230 - protein, 7 aa, Artificial Sequence
  R3 Myelin Basic Protein-binding peptide
TPVLWLP

SEQ ID NO: 231 - protein, 7 aa, Artificial Sequence
  R3 Myelin Basic Protein-binding peptide
TPVRLNT

SEQ ID NO: 232 - protein, 7 aa, Artificial Sequence
  R3 Myelin Basic Protein-binding peptide
TRIPILL

SEQ ID NO: 233 - protein, 7 aa, Artificial Sequence
  R3 Myelin Basic Protein-binding peptide
TTTQGHP

SEQ ID NO: 234 - protein, 7 aa, Artificial Sequence
  R3 Myelin Basic Protein-binding peptide
VGTFWTR

SEQ ID NO: 235 - protein, 7 aa, Artificial Sequence
  R3 Myelin Basic Protein-binding peptide
VIRTAAM

SEQ ID NO: 236 - protein, 7 aa, Artificial Sequence
  R3 Myelin Basic Protein-binding peptide
VLSPYHN

SEQ ID NO: 237 - protein, 7 aa, Artificial Sequence
  R3 Myelin Basic Protein-binding peptide
VRGTPNH

SEQ ID NO: 238 - protein, 7 aa, Artificial Sequence
  R3 Myelin Basic Protein-binding peptide
WDLPQNR

SEQ ID NO: 239 - protein, 7 aa, Artificial Sequence
  R3 Myelin Basic Protein-binding peptide
WDLRHSK

SEQ ID NO: 240 - protein, 7 aa, Artificial Sequence
  R3 Myelin Basic Protein-binding peptide
WTYHPTT

SEQ ID NO: 241 - protein, 7 aa, Artificial Sequence
  R3 Myelin Basic Protein-binding peptide
WVPDRSL

SEQ ID NO: 242 - protein, 7 aa, Artificial Sequence
  R3 Myelin Basic Protein-binding peptide
YPTYLSY

SEQ ID NO: 243 - protein, 7 aa, Artificial Sequence
  R3 Myelin Basic Protein-binding peptide
YPYVPIY

SEQ ID NO: 244 - protein, 7 aa, Artificial Sequence
  R3 Myelin Basic Protein-binding peptide
YSSRLND

SEQ ID NO: 245 - protein, 7 aa, Artificial Sequence
  R3 Myelin Basic Protein-binding peptide
YTASPRW

SEQ ID NO: 246 - protein, 7 aa, Artificial Sequence
  R3 Myelin Basic Protein-binding peptide
YVNRVKQ

SEQ ID NO: 247 - protein, 7 aa, Artificial Sequence
  R3 Myelin Basic Protein-binding peptide
AAGQRSF

SEQ ID NO: 248 - protein, 7 aa, Artificial Sequence
  R3 Myelin Basic Protein-binding peptide
AILTRPP

SEQ ID NO: 249 - protein, 7 aa, Artificial Sequence
  R3 Myelin Basic Protein-binding peptide
ALGTRPL

SEQ ID NO: 250 - protein, 7 aa, Artificial Sequence
  R3 Myelin Basic Protein-binding peptide
ASNTSKG

SEQ ID NO: 251 - protein, 7 aa, Artificial Sequence
  R3 Myelin Basic Protein-binding peptide
AVRQTTM

SEQ ID NO: 252 - protein, 9 aa, Artificial Sequence
  R3 Myelin Basic Protein-binding peptide
CEDSDKSVC

SEQ ID NO: 253 - protein, 9 aa, Artificial Sequence
  R3 Myelin Basic Protein-binding peptide
CNRVPFKQC

SEQ ID NO: 254 - protein, 9 aa, Artificial Sequence
  R3 Myelin Basic Protein-binding peptide
CPRMNNPLC

SEQ ID NO: 255 - protein, 7 aa, Artificial Sequence
  R3 Myelin Basic Protein-binding peptide
DVVKDVI

SEQ ID NO: 256 - protein, 7 aa, Artificial Sequence
  R3 Myelin Basic Protein-binding peptide
ESPYPHS

SEQ ID NO: 257 - protein, 7 aa, Artificial Sequence
  R3 Myelin Basic Protein-binding peptide
FKLPWAS

SEQ ID NO: 258 - protein, 7 aa, Artificial Sequence
  R3 Myelin Basic Protein-binding peptide
FLNQAPT

SEQ ID NO: 259 - protein, 7 aa, Artificial Sequence
  R3 Myelin Basic Protein-binding peptide
FSAGVGK

SEQ ID NO: 260 - protein, 12 aa, Artificial Sequence
  R3 Myelin Basic Protein-binding peptide
GHSHYILGKPTA

SEQ ID NO: 261 - protein, 7 aa, Artificial Sequence
  R3 Myelin Basic Protein-binding peptide
GPITHAI

SEQ ID NO: 262 - protein, 7 aa, Artificial Sequence
  R3 Myelin Basic Protein-binding peptide
HFLRPSD

SEQ ID NO: 263 - protein, 7 aa, Artificial Sequence
  R3 Myelin Basic Protein-binding peptide
HLLKTHS

SEQ ID NO: 264 - protein, 7 aa, Artificial Sequence
  R3 Myelin Basic Protein-binding peptide
HMAGVNQ

SEQ ID NO: 265 - protein, 7 aa, Artificial Sequence
  R3 Myelin Basic Protein-binding peptide
HPDTLPQ

SEQ ID NO: 266 - protein, 7 aa, Artificial Sequence
  R3 Myelin Basic Protein-binding peptide
HPTGTPT

SEQ ID NO: 267 - protein, 7 aa, Artificial Sequence
  R3 Myelin Basic Protein-binding peptide
HTGLRSL

SEQ ID NO: 268 - protein, 7 aa, Artificial Sequence
  R3 Myelin Basic Protein-binding peptide
KGQPLFR

SEQ ID NO: 269 - protein, 7 aa, Artificial Sequence
  R3 Myelin Basic Protein-binding peptide
KYRHGAE

SEQ ID NO: 270 - protein, 7 aa, Artificial Sequence
  R3 Myelin Basic Protein-binding peptide
LFRRVTE

SEQ ID NO: 271 - protein, 7 aa, Artificial Sequence
  R3 Myelin Basic Protein-binding peptide
LTKSLNH

SEQ ID NO: 272 - protein, 7 aa, Artificial Sequence
  R3 Myelin Basic Protein-binding peptide
MPLLELP

SEQ ID NO: 273 - protein, 7 aa, Artificial Sequence
  R3 Myelin Basic Protein-binding peptide
MTNHGNA

SEQ ID NO: 274 - protein, 7 aa, Artificial Sequence
  R3 Myelin Basic Protein-binding peptide
NLKLWTR

SEQ ID NO: 275 - protein, 7 aa, Artificial Sequence
  R3 Myelin Basic Protein-binding peptide
NLLQGVM

SEQ ID NO: 276 - protein, 7 aa, Artificial Sequence
  R3 Myelin Basic Protein-binding peptide
NMYPRVT

SEQ ID NO: 277 - protein, 7 aa, Artificial Sequence
  R3 Myelin Basic Protein-binding peptide
NSSPHQI

SEQ ID NO: 278 - protein, 7 aa, Artificial Sequence
  R3 Myelin Basic Protein-binding peptide
QGTLFDT

SEQ ID NO: 279 - protein, 7 aa, Artificial Sequence
  R3 Myelin Basic Protein-binding peptide
QPIDSTS

SEQ ID NO: 280 - protein, 7 aa, Artificial Sequence
  R3 Myelin Basic Protein-binding peptide
SEPGTVR

SEQ ID NO: 281 - protein, 7 aa, Artificial Sequence
  R3 Myelin Basic Protein-binding peptide
SGNLKKA

SEQ ID NO: 282 - protein, 12 aa, Artificial Sequence
  R3 Myelin Basic Protein-binding peptide
SIAEVLGLWRNV

SEQ ID NO: 283 - protein, 7 aa, Artificial Sequence
  R3 Myelin Basic Protein-binding peptide
SIRMTEI

SEQ ID NO: 284 - protein, 7 aa, Artificial Sequence
  R3 Myelin Basic Protein-binding peptide
SISKIRT

SEQ ID NO: 285 - protein, 7 aa, Artificial Sequence
  R3 Myelin Basic Protein-binding peptide
SLRSVGA

SEQ ID NO: 286 - protein, 7 aa, Artificial Sequence
  R3 Myelin Basic Protein-binding peptide
SSGMLSK

SEQ ID NO: 287 - protein, 7 aa, Artificial Sequence
  R3 Myelin Basic Protein-binding peptide
SYTLSRS

SEQ ID NO: 288 - protein, 7 aa, Artificial Sequence
  R3 Myelin Basic Protein-binding peptide
TLLTALT

SEQ ID NO: 289 - protein, 7 aa, Artificial Sequence
  R3 Myelin Basic Protein-binding peptide
TPLYLSS

SEQ ID NO: 290 - protein, 7 aa, Artificial Sequence
  R3 Myelin Basic Protein-binding peptide
VSFTLEP

SEQ ID NO: 291 - protein, 7 aa, Artificial Sequence
  R3 Myelin Basic Protein-binding peptide
VVDMSTY

SEQ ID NO: 292 - protein, 7 aa, Artificial Sequence
  R3 Myelin Basic Protein-binding peptide
WPDLRIL

SEQ ID NO: 293 - protein, 7 aa, Artificial Sequence
  R3 Myelin Basic Protein-binding peptide
WSLPLLS

SEQ ID NO: 294 - protein, 7 aa, Artificial Sequence
  R3 Myelin Basic Protein-binding peptide
WSPRWPS

SEQ ID NO: 295 - protein, 7 aa, Artificial Sequence
  R3 Myelin Basic Protein-binding peptide
YLPPPLP

SEQ ID NO: 296 - protein, 7 aa, Artificial Sequence
  R3 Myelin Basic Protein-binding peptide
DSYLLSA

SEQ ID NO: 297 - protein, 7 aa, Artificial Sequence
  R3 Myelin Basic Protein-binding peptide
FVLPNKN

SEQ ID NO: 298 - protein, 7 aa, Artificial Sequence
  R3 Myelin Basic Protein-binding peptide
IILPSAQ

SEQ ID NO: 299 - protein, 7 aa, Artificial Sequence
  R3 Myelin Basic Protein-binding peptide
KNSIAPR

SEQ ID NO: 300 - protein, 7 aa, Artificial Sequence
  R3 Myelin Basic Protein-binding peptide
LPPQISR

SEQ ID NO: 301 - protein, 7 aa, Artificial Sequence
  R3 Myelin Basic Protein-binding peptide
MPSLKHQ

SEQ ID NO: 302 - protein, 7 aa, Artificial Sequence
  R3 Myelin Basic Protein-binding peptide
NPTDTNK

SEQ ID NO: 303 - protein, 7 aa, Artificial Sequence
  R3 Myelin Basic Protein-binding peptide
NTSLSFK

SEQ ID NO: 304 - protein, 7 aa, Artificial Sequence
  R3 Myelin Basic Protein-binding peptide
QNSYLSN

SEQ ID NO: 305 - protein, 7 aa, Artificial Sequence
  R3 Myelin Basic Protein-binding peptide
QQAHLQS

SEQ ID NO: 306 - protein, 7 aa, Artificial Sequence
  R3 Myelin Basic Protein-binding peptide
SPPRFIP

SEQ ID NO: 307 - protein, 7 aa, Artificial Sequence
  R3 Myelin Basic Protein-binding peptide
SWFESHN

SEQ ID NO: 308 - protein, 7 aa, Artificial Sequence
  R3 Myelin Basic Protein-binding peptide
TPQVMLK

SEQ ID NO: 309 - protein, 7 aa, Artificial Sequence
  R3 Myelin Basic Protein-binding peptide
TSPPLAH

SEQ ID NO: 310 - protein, 7 aa, Artificial Sequence
  R3 Myelin Basic Protein-binding peptide
TSRLVST

SEQ ID NO: 311 - protein, 7 aa, Artificial Sequence
  R3 Myelin Basic Protein-binding peptide
VAHQRVS

SEQ ID NO: 312 - protein, 7 aa, Artificial Sequence
  R3 Myelin Basic Protein-binding peptide
VPTLRIP

SEQ ID NO: 313 - protein, 7 aa, Artificial Sequence
  R3 Myelin Basic Protein-binding peptide
YATATPS

SEQ ID NO: 314 - protein, 7 aa, Artificial Sequence
  R3 Myelin Basic Protein-binding peptide
ANLSRSV

SEQ ID NO: 315 - protein, 7 aa, Artificial Sequence
  R3 Myelin Basic Protein-binding peptide
FSARTNT

SEQ ID NO: 316 - protein, 7 aa, Artificial Sequence
  R3 Myelin Basic Protein-binding peptide
HFYGPGP

SEQ ID NO: 317 - protein, 7 aa, Artificial Sequence
  R3 Myelin Basic Protein-binding peptide
HPWLGNR

SEQ ID NO: 318 - protein, 7 aa, Artificial Sequence
  R3 Myelin Basic Protein-binding peptide
HYSPNVM

SEQ ID NO: 319 - protein, 7 aa, Artificial Sequence
  R3 Myelin Basic Protein-binding peptide
NFAQHMQ

SEQ ID NO: 320 - protein, 7 aa, Artificial Sequence
  R3 Myelin Basic Protein-binding peptide
SNAERWR

SEQ ID NO: 321 - protein, 7 aa, Artificial Sequence
  R3 Myelin Basic Protein-binding peptide
SRPTRVP

SEQ ID NO: 322 - protein, 7 aa, Artificial Sequence
  R3 Myelin Basic Protein-binding peptide
SVEYGQL

SEQ ID NO: 323 - protein, 7 aa, Artificial Sequence
  R3 Myelin Basic Protein-binding peptide
TGNLRLY

SEQ ID NO: 324 - protein, 7 aa, Artificial Sequence
  R3 Myelin Basic Protein-binding peptide
MDLSLKP

SEQ ID NO: 325 - protein, 7 aa, Artificial Sequence
  R3 Myelin Basic Protein-binding peptide
NTPKVLA

SEQ ID NO: 326 - protein, 7 aa, Artificial Sequence
  R3 Myelin Basic Protein-binding peptide
TQMTMDS

SEQ ID NO: 327 - protein, 7 aa, Artificial Sequence
  Myelin PLP-binding peptide
ASLTLAL

SEQ ID NO: 328 - protein, 7 aa, Artificial Sequence
  Myelin PLP-binding peptide
DRHEGHR

SEQ ID NO: 329 - protein, 7 aa, Artificial Sequence
  Myelin PLP-binding peptide
FRIDKSM

SEQ ID NO: 330 - protein, 7 aa, Artificial Sequence
  Myelin PLP-binding peptide
GHLQSLF

SEQ ID NO: 331 - protein, 7 aa, Artificial Sequence
  Myelin PLP-binding peptide
GLWGPSL

SEQ ID NO: 332 - protein, 7 aa, Artificial Sequence
  Myelin PLP-binding peptide
KPSASIY

SEQ ID NO: 333 - protein, 7 aa, Artificial Sequence
  Myelin PLP-binding peptide
LPSHASI

SEQ ID NO: 334 - protein, 7 aa, Artificial Sequence
  Myelin PLP-binding peptide
LPVKNLL

SEQ ID NO: 335 - protein, 7 aa, Artificial Sequence
  Myelin PLP-binding peptide
MKIMPMD

SEQ ID NO: 336 - protein, 7 aa, Artificial Sequence
  Myelin PLP-binding peptide
SAQSRSF

SEQ ID NO: 337 - protein, 7 aa, Artificial Sequence
  Myelin PLP-binding peptide
SLPSLLP

SEQ ID NO: 338 - protein, 7 aa, Artificial Sequence
  Myelin PLP-binding peptide
SVVYQNS

SEQ ID NO: 339 - protein, 7 aa, Artificial Sequence
  Myelin PLP-binding peptide
TTTIADM

SEQ ID NO: 340 - protein, 7 aa, Artificial Sequence
  Myelin PLP-binding peptide
VTGPGTP

SEQ ID NO: 341 - protein, 7 aa, Artificial Sequence
  Myelin PLP-binding peptide
VTPNKAR

SEQ ID NO: 342 - protein, 7 aa, Artificial Sequence
  Myelin PLP-binding peptide
WHYTPSM

SEQ ID NO: 343 - protein, 7 aa, Artificial Sequence
  Myelin PLP-binding peptide
WSRISVD

SEQ ID NO: 344 - protein, 7 aa, Artificial Sequence
  Myelin PLP-binding peptide
ALFQERK

SEQ ID NO: 345 - protein, 7 aa, Artificial Sequence
  Myelin PLP-binding peptide
AQASSAR

SEQ ID NO: 346 - protein, 7 aa, Artificial Sequence
  Myelin PLP-binding peptide
EVLYPNN

SEQ ID NO: 347 - protein, 7 aa, Artificial Sequence
  Myelin PLP-binding peptide
HLHTRPT

SEQ ID NO: 348 - protein, 7 aa, Artificial Sequence
  Myelin PLP-binding peptide
IRPTHNG

SEQ ID NO: 349 - protein, 7 aa, Artificial Sequence
  Myelin PLP-binding peptide
KGVIPAT

SEQ ID NO: 350 - protein, 7 aa, Artificial Sequence
  Myelin PLP-binding peptide
LHQTYRP

SEQ ID NO: 351 - protein, 7 aa, Artificial Sequence
  Myelin PLP-binding peptide
LPHRLNP

SEQ ID NO: 352 - protein, 7 aa, Artificial Sequence
  Myelin PLP-binding peptide
TGISSTP

SEQ ID NO: 353 - protein, 7 aa, Artificial Sequence
  Myelin PLP-binding peptide
TMPIKAM

SEQ ID NO: 354 - protein, 7 aa, Artificial Sequence
  Myelin PLP-binding peptide
VPGHISG

SEQ ID NO: 355 - protein, 7 aa, Artificial Sequence
  Myelin PLP-binding peptide
VSASWMP

SEQ ID NO: 356 - protein, 7 aa, Artificial Sequence
  Myelin PLP-binding peptide
WSPHGYK

SEQ ID NO: 357 - protein, 7 aa, Artificial Sequence
  Myelin PLP-binding peptide
YSQAISA

SEQ ID NO: 358 - protein, 7 aa, Artificial Sequence
  Myelin PLP-binding peptide
AVVAMNK

SEQ ID NO: 359 - protein, 7 aa, Artificial Sequence
  Myelin PLP-binding peptide
GIPSSKN

SEQ ID NO: 360 - protein, 7 aa, Artificial Sequence
  Myelin PLP-binding peptide
NDASTVS

SEQ ID NO: 361 - protein, 7 aa, Artificial Sequence
  Myelin PLP-binding peptide
QLPRNNL

SEQ ID NO: 362 - protein, 7 aa, Artificial Sequence
  Myelin PLP-binding peptide
SLNRGGA

SEQ ID NO: 363 - protein, 7 aa, Artificial Sequence
  Myelin PLP-binding peptide
SLPGYRH

SEQ ID NO: 364 - protein, 7 aa, Artificial Sequence
  Myelin PLP-binding peptide
SVLPDKL

SEQ ID NO: 365 - protein, 7 aa, Artificial Sequence
  Myelin PLP-binding peptide
TLWAQKT

SEQ ID NO: 366 - protein, 7 aa, Artificial Sequence
  Myelin PLP-binding peptide
TQPRYPS

SEQ ID NO: 367 - protein, 7 aa, Artificial Sequence
  Myelin PLP-binding peptide
VFPERRV

SEQ ID NO: 368 - protein, 7 aa, Artificial Sequence
  Myelin PLP-binding peptide
VGYRSAS

SEQ ID NO: 369 - protein, 7 aa, Artificial Sequence
  Myelin PLP-binding peptide
VRTSMNH

SEQ ID NO: 370 - protein, 7 aa, Artificial Sequence
  Myelin PLP-binding peptide
YTWTPSR

SEQ ID NO: 371 - protein, 7 aa, Artificial Sequence
  Myelin PLP-binding peptide
FSLDRDG

SEQ ID NO: 372 - protein, 7 aa, Artificial Sequence
  Myelin PLP-binding peptide
FSTPTNV

SEQ ID NO: 373 - protein, 7 aa, Artificial Sequence
  Myelin PLP-binding peptide
GPAAVII

SEQ ID NO: 374 - protein, 7 aa, Artificial Sequence
  Myelin PLP-binding peptide
GWSTAIR

SEQ ID NO: 375 - protein, 7 aa, Artificial Sequence
  Myelin PLP-binding peptide
HKAPLGT

SEQ ID NO: 376 - protein, 7 aa, Artificial Sequence
  Myelin PLP-binding peptide
IDRVRGL

SEQ ID NO: 377 - protein, 7 aa, Artificial Sequence
  Myelin PLP-binding peptide
LMQKPSI

SEQ ID NO: 378 - protein, 7 aa, Artificial Sequence
  Myelin PLP-binding peptide
SVMWMTP

SEQ ID NO: 379 - protein, 7 aa, Artificial Sequence
  Myelin PLP-binding peptide
TKITPHR

SEQ ID NO: 380 - protein, 7 aa, Artificial Sequence
  Myelin PLP-binding peptide
YSSRLTA

SEQ ID NO: 381 - protein, 7 aa, Artificial Sequence
  Myelin PLP-binding peptide
ARFVPLT

SEQ ID NO: 382 - protein, 7 aa, Artificial Sequence
  Myelin PLP-binding peptide
DGSRDLV

SEQ ID NO: 383 - protein, 7 aa, Artificial Sequence
  Myelin PLP-binding peptide
EKSTTVA

SEQ ID NO: 384 - protein, 7 aa, Artificial Sequence
  Myelin PLP-binding peptide
YSDTYKH

SEQ ID NO: 385 - protein, 7 aa, Artificial Sequence
  Myelin PLP-binding peptide
EPSSFTF

SEQ ID NO: 386 - protein, 7 aa, Artificial Sequence
  Myelin PLP-binding peptide
GKSYSQI

SEQ ID NO: 387 - protein, 7 aa, Artificial Sequence
  Myelin PLP-binding peptide
GNVFQTS

SEQ ID NO: 388 - protein, 7 aa, Artificial Sequence
  Myelin PLP-binding peptide
IIPPTSV

SEQ ID NO: 389 - protein, 7 aa, Artificial Sequence
  Myelin PLP-binding peptide
LPLRLHA

SEQ ID NO: 390 - protein, 7 aa, Artificial Sequence
  Myelin PLP-binding peptide
NVFSATP

SEQ ID NO: 391 - protein, 7 aa, Artificial Sequence
  Myelin PLP-binding peptide
QRRSVIL

SEQ ID NO: 392 - protein, 7 aa, Artificial Sequence
  Myelin PLP-binding peptide
SGVATYT

SEQ ID NO: 393 - protein, 7 aa, Artificial Sequence
  Myelin PLP-binding peptide
SWDVLEL

SEQ ID NO: 394 - protein, 7 aa, Artificial Sequence
  Myelin PLP-binding peptide
TFPERLR

SEQ ID NO: 395 - protein, 7 aa, Artificial Sequence
  Myelin PLP-binding peptide
WSPHRSF

SEQ ID NO: 396 - protein, 7 aa, Artificial Sequence
  Myelin PLP-binding peptide
IHHLESS

SEQ ID NO: 397 - protein, 7 aa, Artificial Sequence
  Myelin PLP-binding peptide
LNGISAL

SEQ ID NO: 398 - protein, 7 aa, Artificial Sequence
  Myelin PLP-binding peptide
SALISTR

SEQ ID NO: 399 - protein, 7 aa, Artificial Sequence
  Myelin PLP-binding peptide
SGLHYAL

SEQ ID NO: 400 - protein, 7 aa, Artificial Sequence
  Myelin PLP-binding peptide
TRASYPQ

SEQ ID NO: 401 - protein, 7 aa, Artificial Sequence
  Myelin PLP-binding peptide
NIPSSIL

SEQ ID NO: 402 - protein, 7 aa, Artificial Sequence
  Myelin PLP-binding peptide
QPNMLKP

SEQ ID NO: 403 - protein, 7 aa, Artificial Sequence
  Myelin PLP-binding peptide
RLQPEPT

SEQ ID NO: 404 - protein, 7 aa, Artificial Sequence
  Myelin PLP-binding peptide
SVEKYSK

SEQ ID NO: 405 - protein, 7 aa, Artificial Sequence
  Myelin PLP-binding peptide
VSSQEQA

SEQ ID NO: 406 - protein, 7 aa, Artificial Sequence
  Myelin PLP-binding peptide
WNTADRL

SEQ ID NO: 407 - protein, 7 aa, Artificial Sequence
  Myelin PLP-binding peptide
MPHAPVQ

SEQ ID NO: 408 - protein, 7 aa, Artificial Sequence
  Myelin PLP-binding peptide
TDVEMVP

SEQ ID NO: 409 - protein, 7 aa, Artificial Sequence
  Myelin PLP-binding peptide
AHQSSVT

SEQ ID NO: 410 - protein, 7 aa, Artificial Sequence
  Myelin PLP-binding peptide
GSAWKKH

SEQ ID NO: 411 - protein, 9 aa, Artificial Sequence
  Nidogen-2-binding peptide
CIHHKGVLC

SEQ ID NO: 412 - protein, 9 aa, Artificial Sequence
  Nidogen-2-binding peptide
CKDGHIRHC

SEQ ID NO: 413 - protein, 9 aa, Artificial Sequence
  Nidogen-2-binding peptide
CNLATSLQC

SEQ ID NO: 414 - protein, 9 aa, Artificial Sequence
  Nidogen-2-binding peptide
CRSPHEPMC

SEQ ID NO: 415 - protein, 9 aa, Artificial Sequence
  Nidogen-2-binding peptide
CVDKMASVC

SEQ ID NO: 416 - protein, 9 aa, Artificial Sequence
  Nidogen-2-binding peptide
CYSAPTKSC

SEQ ID NO: 417 - protein, 7 aa, Artificial Sequence
  Nidogen-2-binding peptide
EAMMHRN

SEQ ID NO: 418 - protein, 7 aa, Artificial Sequence
  Nidogen-2-binding peptide
LHITPEV

SEQ ID NO: 419 - protein, 7 aa, Artificial Sequence
  Nidogen-2-binding peptide
LSIRGLT

SEQ ID NO: 420 - protein, 7 aa, Artificial Sequence
  Nidogen-2-binding peptide
NILSQVN

SEQ ID NO: 421 - protein, 7 aa, Artificial Sequence
  Nidogen-2-binding peptide
QLLESRT

SEQ ID NO: 422 - protein, 7 aa, Artificial Sequence
  Nidogen-2-binding peptide
QMDAKHM

SEQ ID NO: 423 - protein, 7 aa, Artificial Sequence
  Nidogen-2-binding peptide
QTLRPKQ

SEQ ID NO: 424 - protein, 7 aa, Artificial Sequence
  Nidogen-2-binding peptide
TIASLLV

SEQ ID NO: 425 - protein, 7 aa, Artificial Sequence
  Nidogen-2-binding peptide
WLAAGSQ

SEQ ID NO: 426 - protein, 7 aa, Artificial Sequence
  Nidogen-2-binding peptide
ALYLPGR

SEQ ID NO: 427 - protein, 7 aa, Artificial Sequence
  Nidogen-2-binding peptide
ARMAFSL

SEQ ID NO: 428 - protein, 7 aa, Artificial Sequence
  Nidogen-2-binding peptide
ATYNMSQ

SEQ ID NO: 429 - protein, 7 aa, Artificial Sequence
  Nidogen-2-binding peptide
GLRTMEP

SEQ ID NO: 430 - protein, 7 aa, Artificial Sequence
  Nidogen-2-binding peptide
TPWLPTI

SEQ ID NO: 431 - protein, 7 aa, Artificial Sequence
  Nidogen-2-binding peptide
YNHTMMY

SEQ ID NO: 432 - protein, 9 aa, Artificial Sequence
  Nidogen-2-binding peptide
CSFKMNQKC

SEQ ID NO: 433 - protein, 7 aa, Artificial Sequence
  Nidogen-2-binding peptide
LVHPFHG

SEQ ID NO: 434 - protein, 9 aa, Artificial Sequence
  Nidogen-2-binding peptide
CSSSAPRIC

SEQ ID NO: 435 - protein, 7 aa, Artificial Sequence
  Nidogen-2-binding peptide
HSRLPTP

SEQ ID NO: 436 - protein, 9 aa, Artificial Sequence
  Laminin 421/521-binding peptide
CKSPIKGTC

SEQ ID NO: 437 - protein, 9 aa, Artificial Sequence
  Laminin 421/521-binding peptide
CLQKNHKFC

SEQ ID NO: 438 - protein, 12 aa, Artificial Sequence
  Laminin 421/521-binding peptide
CVLKCEDQKYRG

SEQ ID NO: 439 - protein, 12 aa, Artificial Sequence
  Laminin 421/521-binding peptide
DGANMFNIAPAN

SEQ ID NO: 440 - protein, 12 aa, Artificial Sequence
  Laminin 421/521-binding peptide
DRMYSTVPAEGL

SEQ ID NO: 441 - protein, 7 aa, Artificial Sequence
  Laminin 421/521-binding peptide
EAVSTQI

SEQ ID NO: 442 - protein, 12 aa, Artificial Sequence
  Laminin 421/521-binding peptide
GINLRALDLHAN

SEQ ID NO: 443 - protein, 12 aa, Artificial Sequence
  Laminin 421/521-binding peptide
HQSVTGVRSHFH

SEQ ID NO: 444 - protein, 12 aa, Artificial Sequence
  Laminin 421/521-binding peptide
SVVGWAAPRTAQ

SEQ ID NO: 445 - protein, 12 aa, Artificial Sequence
  Laminin 421/521-binding peptide
TILKPAAQGFAD

SEQ ID NO: 446 - protein, 7 aa, Artificial Sequence
  Laminin 421/521-binding peptide
TSAVSLR

SEQ ID NO: 447 - protein, 12 aa, Artificial Sequence
  Laminin 421/521-binding peptide
TTLWLDRDEALK

SEQ ID NO: 448 - protein, 12 aa, Artificial Sequence
  Laminin 421/521-binding peptide
VIADQSKSAVAV

SEQ ID NO: 449 - protein, 12 aa, Artificial Sequence
  Laminin 421/521-binding peptide
AQNLRVHAWASL

SEQ ID NO: 450 - protein, 9 aa, Artificial Sequence
  Laminin 421/521-binding peptide
CPMHQSKTC

SEQ ID NO: 451 - protein, 9 aa, Artificial Sequence
  Laminin 421/521-binding peptide
CTDGRNFVC

SEQ ID NO: 452 - protein, 9 aa, Artificial Sequence
  Laminin 421/521-binding peptide
CTGETLLTC

SEQ ID NO: 453 - protein, 9 aa, Artificial Sequence
  Laminin 421/521-binding peptide
CTSSSHQTC

SEQ ID NO: 454 - protein, 12 aa, Artificial Sequence
  Laminin 421/521-binding peptide
ETGGLAYGSGQK

SEQ ID NO: 455 - protein, 12 aa, Artificial Sequence
  Laminin 421/521-binding peptide
GIVPSRHTTGLG

SEQ ID NO: 456 - protein, 12 aa, Artificial Sequence
  Laminin 421/521-binding peptide
GWTSDLSRNVRG

SEQ ID NO: 457 - protein, 7 aa, Artificial Sequence
  Laminin 421/521-binding peptide
HTQIHRL

SEQ ID NO: 458 - protein, 12 aa, Artificial Sequence
  Laminin 421/521-binding peptide
KFADTKLTSLRY

SEQ ID NO: 459 - protein, 7 aa, Artificial Sequence
  Laminin 421/521-binding peptide
QLQHVHL

SEQ ID NO: 460 - protein, 12 aa, Artificial Sequence
  Laminin 421/521-binding peptide
QPTPSQIKFTRT

SEQ ID NO: 461 - protein, 12 aa, Artificial Sequence
  Laminin 421/521-binding peptide
RALDRYLPWSPH

SEQ ID NO: 462 - protein, 12 aa, Artificial Sequence
  Laminin 421/521-binding peptide
SLPYAASLNSVE

SEQ ID NO: 463 - protein, 7 aa, Artificial Sequence
  Laminin 421/521-binding peptide
STNVQRA

SEQ ID NO: 464 - protein, 12 aa, Artificial Sequence
  Laminin 421/521-binding peptide
SVLSTGTANQRH

SEQ ID NO: 465 - protein, 12 aa, Artificial Sequence
  Laminin 421/521-binding peptide
SVQTPMWRSLVG

SEQ ID NO: 466 - protein, 12 aa, Artificial Sequence
  Laminin 421/521-binding peptide
SWHFTGTPFMNR

SEQ ID NO: 467 - protein, 12 aa, Artificial Sequence
  Laminin 421/521-binding peptide
SYTATWSEMSRS

SEQ ID NO: 468 - protein, 12 aa, Artificial Sequence
  Laminin 421/521-binding peptide
TALSHRHEAMRW

SEQ ID NO: 469 - protein, 7 aa, Artificial Sequence
  Laminin 421/521-binding peptide
TKGIAPK

SEQ ID NO: 470 - protein, 12 aa, Artificial Sequence
  Laminin 421/521-binding peptide
TVARSTAQERSI

SEQ ID NO: 471 - protein, 12 aa, Artificial Sequence
  Laminin 421/521-binding peptide
AVRPLGLPDNHR

SEQ ID NO: 472 - protein, 9 aa, Artificial Sequence
  Laminin 421/521-binding peptide
CIGGPHRNC

SEQ ID NO: 473 - protein, 9 aa, Artificial Sequence
  Laminin 421/521-binding peptide
CKSPAIKGC

SEQ ID NO: 474 - protein, 9 aa, Artificial Sequence
  Laminin 421/521-binding peptide
CNSWKAAKC

SEQ ID NO: 475 - protein, 12 aa, Artificial Sequence
  Laminin 421/521-binding peptide
DSLYRGMHQPRI

SEQ ID NO: 476 - protein, 12 aa, Artificial Sequence
  Laminin 421/521-binding peptide
EDRMMTYRYTST

SEQ ID NO: 477 - protein, 12 aa, Artificial Sequence
  Laminin 421/521-binding peptide
EVERILTPHVNN

SEQ ID NO: 478 - protein, 12 aa, Artificial Sequence
  Laminin 421/521-binding peptide
FNSDSRSTHQED

SEQ ID NO: 479 - protein, 12 aa, Artificial Sequence
  Laminin 421/521-binding peptide
HPAWADFFTMSS

SEQ ID NO: 480 - protein, 7 aa, Artificial Sequence
  Laminin 421/521-binding peptide
IDLSLRS

SEQ ID NO: 481 - protein, 12 aa, Artificial Sequence
  Laminin 421/521-binding peptide
IDQSGLQKSGMK

SEQ ID NO: 482 - protein, 12 aa, Artificial Sequence
  Laminin 421/521-binding peptide
LGSSRSPSSFLG

SEQ ID NO: 483 - protein, 12 aa, Artificial Sequence
  Laminin 421/521-binding peptide
NGHEVQSRAANR

SEQ ID NO: 484 - protein, 12 aa, Artificial Sequence
  Laminin 421/521-binding peptide
QGWKDRLPIWRY

SEQ ID NO: 485 - protein, 12 aa, Artificial Sequence
  Laminin 421/521-binding peptide
SHDVANGTSVRT

SEQ ID NO: 486 - protein, 12 aa, Artificial Sequence
  Laminin 421/521-binding peptide
SPDRTNWGWQTN

SEQ ID NO: 487 - protein, 12 aa, Artificial Sequence
  Laminin 421/521-binding peptide
SVESNSKLTMPR

SEQ ID NO: 488 - protein, 7 aa, Artificial Sequence
  Laminin 421/521-binding peptide
WPMMQTR

SEQ ID NO: 489 - protein, 9 aa, Artificial Sequence
  Laminin 421/521-binding peptide
CKSERGPEC

SEQ ID NO: 490 - protein, 9 aa, Artificial Sequence
  Laminin 421/521-binding peptide
CQGWPRPMC

SEQ ID NO: 491 - protein, 9 aa, Artificial Sequence
  Laminin 421/521-binding peptide
CSRAGLSAC

SEQ ID NO: 492 - protein, 9 aa, Artificial Sequence
  Laminin 421/521-binding peptide
CTWKHRDNC

SEQ ID NO: 493 - protein, 12 aa, Artificial Sequence
  Laminin 421/521-binding peptide
GLLDWGSLQGGN

SEQ ID NO: 494 - protein, 7 aa, Artificial Sequence
  Laminin 421/521-binding peptide
GPLLFRG

SEQ ID NO: 495 - protein, 7 aa, Artificial Sequence
  Laminin 421/521-binding peptide
GSPEKRT

SEQ ID NO: 496 - protein, 7 aa, Artificial Sequence
  Laminin 421/521-binding peptide
KASIAYD

SEQ ID NO: 497 - protein, 12 aa, Artificial Sequence
  Laminin 421/521-binding peptide
LGYGQGTPHRSN

SEQ ID NO: 498 - protein, 12 aa, Artificial Sequence
  Laminin 421/521-binding peptide
QGWEFQVPARHS

SEQ ID NO: 499 - protein, 12 aa, Artificial Sequence
  Laminin 421/521-binding peptide
QTFGYSMFNVRT

SEQ ID NO: 500 - protein, 12 aa, Artificial Sequence
  Laminin 421/521-binding peptide
RNLHYPLNHPFM

SEQ ID NO: 501 - protein, 7 aa, Artificial Sequence
  Laminin 421/521-binding peptide
TAPRWTQ

SEQ ID NO: 502 - protein, 7 aa, Artificial Sequence
  Laminin 421/521-binding peptide
VPSYILR

SEQ ID NO: 503 - protein, 7 aa, Artificial Sequence
  Laminin 421/521-binding peptide
WRSPLTT

SEQ ID NO: 504 - protein, 9 aa, Artificial Sequence
  Laminin 421/521-binding peptide
CQTSGHHQC

SEQ ID NO: 505 - protein, 9 aa, Artificial Sequence
  Laminin 421/521-binding peptide
CSSQAKRSC

SEQ ID NO: 506 - protein, 9 aa, Artificial Sequence
  Laminin 421/521-binding peptide
CTAWTRQEC

SEQ ID NO: 507 - protein, 12 aa, Artificial Sequence
  Laminin 421/521-binding peptide
EYVKFSHSPRTY

SEQ ID NO: 508 - protein, 12 aa, Artificial Sequence
  Laminin 421/521-binding peptide
GGWQSLWDKPEH

SEQ ID NO: 509 - protein, 12 aa, Artificial Sequence
  Laminin 421/521-binding peptide
GPMLKNLSDAVT

SEQ ID NO: 510 - protein, 12 aa, Artificial Sequence
  Laminin 421/521-binding peptide
NHDQGSLTRWRS

SEQ ID NO: 511 - protein, 12 aa, Artificial Sequence
  Laminin 421/521-binding peptide
SLLSVVNTSSKS

SEQ ID NO: 512 - protein, 12 aa, Artificial Sequence
  Laminin 421/521-binding peptide
VVPSWPSVHRPP

SEQ ID NO: 513 - protein, 12 aa, Artificial Sequence
  Laminin 421/521-binding peptide
WGWNGANMSPRG

SEQ ID NO: 514 - protein, 9 aa, Artificial Sequence
  Laminin 421/521-binding peptide
CHNADNNGC

SEQ ID NO: 515 - protein, 7 aa, Artificial Sequence
  Laminin 421/521-binding peptide
LPMLRHS

SEQ ID NO: 516 - protein, 12 aa, Artificial Sequence
  Laminin 421/521-binding peptide
NDTGRHASGISK

SEQ ID NO: 517 - protein, 12 aa, Artificial Sequence
  Laminin 421/521-binding peptide
QALRTNYSPLNS

SEQ ID NO: 518 - protein, 12 aa, Artificial Sequence
  Laminin 421/521-binding peptide
TLKAGTRANDGV

SEQ ID NO: 519 - protein, 9 aa, Artificial Sequence
  Laminin 421/521-binding peptide
CGETTAGKC

SEQ ID NO: 520 - protein, 7 aa, Artificial Sequence
  Laminin 421/521-binding peptide
HSTYAPG

SEQ ID NO: 521 - protein, 12 aa, Artificial Sequence
  Laminin 421/521-binding peptide
QINWKQADKNAD

SEQ ID NO: 522 - protein, 12 aa, Artificial Sequence
  Laminin 421/521-binding peptide
ALPWQSGVHGTK

SEQ ID NO: 523 - protein, 12 aa, Artificial Sequence
  Laminin 421/521-binding peptide
TFGSGRSMPIQY

SEQ ID NO: 524 - protein, 12 aa, Artificial Sequence
  Laminin 421/521-binding peptide
VYRNGGGLPLTA

SEQ ID NO: 525 - protein, 12 aa, Artificial Sequence
  Laminin 421/521-binding peptide
ALKIGPETTIYM

SEQ ID NO: 526 - protein, 12 aa, Artificial Sequence
  Laminin 421/521-binding peptide
DWTQVRVTNWFL

SEQ ID NO: 527 - protein, 12 aa, Artificial Sequence
  Laminin 421/521-binding peptide
LGHQNGGRADMW

SEQ ID NO: 528 - protein, 7 aa, Artificial Sequence
  Laminin 421/521-binding peptide
MTNSGAK

SEQ ID NO: 529 - protein, 7 aa, Artificial Sequence
  Laminin 421/521-binding peptide
WAYTDYM

SEQ ID NO: 530 - protein, 12 aa, Artificial Sequence
  Laminin 421/521-binding peptide
AHAAGRDMRQGT

SEQ ID NO: 531 - protein, 12 aa, Artificial Sequence
  Laminin 421/521-binding peptide
DVIHSSRGAYFE

SEQ ID NO: 532 - protein, 12 aa, Artificial Sequence
  Laminin 421/521-binding peptide
NWTHLGVARLQP

SEQ ID NO: 533 - protein, 12 aa, Artificial Sequence
  Laminin 421/521-binding peptide
SYDGTMLKQVRL

SEQ ID NO: 534 - protein, 12 aa, Artificial Sequence
  Laminin 421/521-binding peptide
WQWRSTELGYRY

SEQ ID NO: 535 - protein, 12 aa, Artificial Sequence
  Laminin 421/521-binding peptide
WSPANGWRHQTI

SEQ ID NO: 536 - protein, 9 aa, Artificial Sequence
  Laminin 421/521-binding peptide
CLHNGQRSC

SEQ ID NO: 537 - protein, 9 aa, Artificial Sequence
  Laminin 421/521-binding peptide
CTPRSATLC

SEQ ID NO: 538 - protein, 12 aa, Artificial Sequence
  Laminin 421/521-binding peptide
DIMKSPRSNLRS

SEQ ID NO: 539 - protein, 12 aa, Artificial Sequence
  Laminin 421/521-binding peptide
QTFHEYLNPARG

SEQ ID NO: 540 - protein, 12 aa, Artificial Sequence
  Laminin 421/521-binding peptide
SLNTTWVSPMMK

SEQ ID NO: 541 - protein, 7 aa, Artificial Sequence
  Laminin 421/521-binding peptide
AQRMNQA

SEQ ID NO: 542 - protein, 7 aa, Artificial Sequence
  Laminin 421/521-binding peptide
GDAQILM

SEQ ID NO: 543 - protein, 12 aa, Artificial Sequence
  Laminin 421/521-binding peptide
GHRTLVTSERYL

SEQ ID NO: 544 - protein, 12 aa, Artificial Sequence
  Laminin 421/521-binding peptide
LGPSTQGAGQTR

SEQ ID NO: 545 - protein, 12 aa, Artificial Sequence
  Laminin 421/521-binding peptide
TFAAAQAELIMV

SEQ ID NO: 546 - protein, 12 aa, Artificial Sequence
  Laminin 421/521-binding peptide
WEEHRVEILPDV

SEQ ID NO: 547 - protein, 12 aa, Artificial Sequence
  Laminin 421/521-binding peptide
AGAYTSRHAFDE

SEQ ID NO: 548 - protein, 9 aa, Artificial Sequence
  Laminin 421/521-binding peptide
CGLATNKSC

SEQ ID NO: 549 - protein, 12 aa, Artificial Sequence
  Laminin 421/521-binding peptide
GDVITAYINPWP

SEQ ID NO: 550 - protein, 12 aa, Artificial Sequence
  Laminin 421/521-binding peptide
GYDLSRLWGMAS

SEQ ID NO: 551 - protein, 7 aa, Artificial Sequence
  Laminin 421/521-binding peptide
ANRYLAS

SEQ ID NO: 552 - protein, 9 aa, Artificial Sequence
  Laminin 421/521-binding peptide
CKWTQLWGC

SEQ ID NO: 553 - protein, 9 aa, Artificial Sequence
  Laminin 421/521-binding peptide
CLKDTHLNC

SEQ ID NO: 554 - protein, 12 aa, Artificial Sequence
  Laminin 421/521-binding peptide
GQPRNIHLPGTH

SEQ ID NO: 555 - protein, 12 aa, Artificial Sequence
  Laminin 421/521-binding peptide
GSTTHPHFGLPG

SEQ ID NO: 556 - protein, 12 aa, Artificial Sequence
  Laminin 421/521-binding peptide
GWIQDTFVLGRS

SEQ ID NO: 557 - protein, 7 aa, Artificial Sequence
  Laminin 421/521-binding peptide
QGHPFIY

SEQ ID NO: 558 - protein, 12 aa, Artificial Sequence
  Laminin 421/521-binding peptide
SNPKHVSSLGQM

SEQ ID NO: 559 - protein, 12 aa, Artificial Sequence
  Laminin 421/521-binding peptide
WGANTTGRTHGG

SEQ ID NO: 560 - protein, 12 aa, Artificial Sequence
  Laminin 421/521-binding peptide
AQGVWWSEWFAP

SEQ ID NO: 561 - protein, 9 aa, Artificial Sequence
  Laminin 421/521-binding peptide
CFQPKMNSC

SEQ ID NO: 562 - protein, 9 aa, Artificial Sequence
  Laminin 421/521-binding peptide
CWSGNSRSC

SEQ ID NO: 563 - protein, 7 aa, Artificial Sequence
  Laminin 421/521-binding peptide
FPLKIRT

SEQ ID NO: 564 - protein, 12 aa, Artificial Sequence
  Laminin 421/521-binding peptide
LARSSIMAANNV

SEQ ID NO: 565 - protein, 12 aa, Artificial Sequence
  Laminin 421/521-binding peptide
AKTQAPSNWSGV

SEQ ID NO: 566 - protein, 9 aa, Artificial Sequence
  Laminin 421/521-binding peptide
CSVWNSGNC

SEQ ID NO: 567 - protein, 9 aa, Artificial Sequence
  Laminin 421/521-binding peptide
CTGRISKHC

SEQ ID NO: 568 - protein, 12 aa, Artificial Sequence
  Laminin 421/521-binding peptide
GAMMNSNNLVAR

SEQ ID NO: 569 - protein, 12 aa, Artificial Sequence
  Laminin 421/521-binding peptide
GGGYHIFGPLVT

SEQ ID NO: 570 - protein, 7 aa, Artificial Sequence
  Laminin 421/521-binding peptide
HSFLGAR

SEQ ID NO: 571 - protein, 7 aa, Artificial Sequence
  Laminin 421/521-binding peptide
LLAGPFR

SEQ ID NO: 572 - protein, 7 aa, Artificial Sequence
  Laminin 421/521-binding peptide
QTLQTRH

SEQ ID NO: 573 - protein, 12 aa, Artificial Sequence
  Laminin 421/521-binding peptide
ALGGEPRKAYQR

SEQ ID NO: 574 - protein, 9 aa, Artificial Sequence
  Laminin 421/521-binding peptide
CNHLTQKLC

SEQ ID NO: 575 - protein, 9 aa, Artificial Sequence
  Laminin 421/521-binding peptide
CTSKLARHC

SEQ ID NO: 576 - protein, 12 aa, Artificial Sequence
  Laminin 421/521-binding peptide
AVYPLDLGAGMR

SEQ ID NO: 577 - protein, 9 aa, Artificial Sequence
  Laminin 421/521-binding peptide
CHRTYNSTC

SEQ ID NO: 578 - protein, 12 aa, Artificial Sequence
  Laminin 421/521-binding peptide
LDRNADSVRAVL

SEQ ID NO: 579 - protein, 12 aa, Artificial Sequence
  Laminin 421/521-binding peptide
TTNGVPGHDRSP

SEQ ID NO: 580 - protein, 12 aa, Artificial Sequence
  Laminin 421/521-binding peptide
ADMPQMTLKYGV

SEQ ID NO: 581 - protein, 7 aa, Artificial Sequence
  Laminin 421/521-binding peptide
AWNGNRS

SEQ ID NO: 582 - protein, 7 aa, Artificial Sequence
  Laminin 421/521-binding peptide
GDLLLKR

SEQ ID NO: 583 - protein, 7 aa, Artificial Sequence
  Laminin 421/521-binding peptide
VPQIRIK

SEQ ID NO: 584 - protein, 7 aa, Artificial Sequence
  Laminin 421/521-binding peptide
SWQMRSY

SEQ ID NO: 585 - protein, 12 aa, Artificial Sequence
  Laminin 421/521-binding peptide
YHLGPNQKMRTS

SEQ ID NO: 586 - protein, 12 aa, Artificial Sequence
  Laminin 421/521-binding peptide
ASHSRPLMNYAP

SEQ ID NO: 587 - protein, 12 aa, Artificial Sequence
  Laminin 421/521-binding peptide
QTRWDDGSYQIS

SEQ ID NO: 588 - protein, 12 aa, Artificial Sequence
  Laminin 421/521-binding peptide
SLAVANTRFMIR

SEQ ID NO: 589 - protein, 12 aa, Artificial Sequence
  Laminin 421/521-binding peptide
EGHVWSEYTWGT

SEQ ID NO: 590 - protein, 12 aa, Artificial Sequence
  Laminin 421/521-binding peptide
ETYKVTRVISPW

SEQ ID NO: 591 - protein, 12 aa, Artificial Sequence
  Laminin 421/521-binding peptide
GHPWTEIDFMSS

SEQ ID NO: 592 - protein, 12 aa, Artificial Sequence
  Laminin 421/521-binding peptide
KQELSDNLASHR

SEQ ID NO: 593 - protein, 12 aa, Artificial Sequence
  Laminin 421/521-binding peptide
SFRNLEKLSLWS

SEQ ID NO: 594 - protein, 7 aa, Artificial Sequence
  Laminin 421/521-binding peptide
GDLWNVP

SEQ ID NO: 595 - protein, 7 aa, Artificial Sequence
  Laminin 421/521-binding peptide
GPKNHHQ

SEQ ID NO: 596 - protein, 12 aa, Artificial Sequence
  Laminin 421/521-binding peptide
GSGFRNEEHSAH

SEQ ID NO: 597 - protein, 12 aa, Artificial Sequence
  Laminin 421/521-binding peptide
NNNAAMQNHGVR

SEQ ID NO: 598 - protein, 7 aa, Artificial Sequence
  Laminin 421/521-binding peptide
RAQAHQV

SEQ ID NO: 599 - protein, 9 aa, Artificial Sequence
  Laminin 421/521-binding peptide
CSLANPATC

SEQ ID NO: 600 - protein, 12 aa, Artificial Sequence
  Laminin 421/521-binding peptide
GEPLTRFPNSDS

SEQ ID NO: 601 - protein, 12 aa, Artificial Sequence
  Laminin 421/521-binding peptide
KPILWNRSLNAL

SEQ ID NO: 602 - protein, 12 aa, Artificial Sequence
  Laminin 421/521-binding peptide
GFADVIHRKWSS

SEQ ID NO: 603 - protein, 12 aa, Artificial Sequence
  Laminin 421/521-binding peptide
HQSLAGVPWSRH

SEQ ID NO: 604 - protein, 12 aa, Artificial Sequence
  Laminin 421/521-binding peptide
VPVFLDQARVMK

SEQ ID NO: 605 - protein, 12 aa, Artificial Sequence
  Laminin 421/521-binding peptide
DDLLTAPRLGVW

SEQ ID NO: 606 - protein, 12 aa, Artificial Sequence
  Laminin 421/521-binding peptide
SDRGKTVLHGQI

SEQ ID NO: 607 - protein, 9 aa, Artificial Sequence
  Laminin 421/521-binding peptide
CGITQTTTC

SEQ ID NO: 608 - protein, 9 aa, Artificial Sequence
  Laminin 421/521-binding peptide
CHQRQQTYC

SEQ ID NO: 609 - protein, 12 aa, Artificial Sequence
  Laminin 421/521-binding peptide
EYWHQRGSWFHR

SEQ ID NO: 610 - protein, 7 aa, Artificial Sequence
  Laminin 421/521-binding peptide
YTPRNQL

SEQ ID NO: 611 - protein, 9 aa, Artificial Sequence
  Laminin 421/521-binding peptide
CTKSPSNSC

SEQ ID NO: 612 - protein, 7 aa, Artificial Sequence
  Laminin 421/521-binding peptide
WAFKAPQ

SEQ ID NO: 613 - protein, 12 aa, Artificial Sequence
  Laminin 421/521-binding peptide
GSLDMGNNKQPV

SEQ ID NO: 614 - protein, 12 aa, Artificial Sequence
  Laminin 421/521-binding peptide
AVVSQNQMSQQK

SEQ ID NO: 615 - protein, 9 aa, Artificial Sequence
  Laminin 421/521-binding peptide
CLNQKWEAC

SEQ ID NO: 616 - protein, 7 aa, Artificial Sequence
  Laminin 421/521-binding peptide
WPVRNLL

SEQ ID NO: 617 - protein, 12 aa, Artificial Sequence
  Laminin 421/521-binding peptide
GYRSFIHENWSI

SEQ ID NO: 618 - protein, 12 aa, Artificial Sequence
  Laminin 421/521-binding peptide
SGWEGKTHQGVR

SEQ ID NO: 619 - protein, 12 aa, Artificial Sequence
  Laminin 421/521-binding peptide
LQSNAADHHQGM

SEQ ID NO: 620 - protein, 12 aa, Artificial Sequence
  Laminin 421/521-binding peptide
ATSKYPNSWAQT

SEQ ID NO: 621 - protein, 12 aa, Artificial Sequence
  Laminin 421/521-binding peptide
DRLTPITWDWSR

SEQ ID NO: 622 - protein, 12 aa, Artificial Sequence
  Laminin 421/521-binding peptide
WTKHNTPAHHLS

SEQ ID NO: 623 - protein, 12 aa, Artificial Sequence
  Laminin 421/521-binding peptide
WNANYGTMTRHA

SEQ ID NO: 624 - protein, 7 aa, Artificial Sequence
  Laminin 421/521-binding peptide
HASNQRV

SEQ ID NO: 625 - protein, 7 aa, Artificial Sequence
  Laminin 421/521-binding peptide
GRTIHSM

SEQ ID NO: 626 - protein, 12 aa, Artificial Sequence
  Laminin 421/521-binding peptide
RLVTASMVSPSF

SEQ ID NO: 627 - protein, 12 aa, Artificial Sequence
  Laminin 421/521-binding peptide
DGQVSDHSYVQW

SEQ ID NO: 628 - protein, 12 aa, Artificial Sequence
  Laminin 421/521-binding peptide
KISMNRLHANFT

SEQ ID NO: 629 - protein, 12 aa, Artificial Sequence
  Laminin 421/521-binding peptide
WDLPTLRKTKQA

SEQ ID NO: 630 - protein, 7 aa, Artificial Sequence
  Laminin 421/521-binding peptide
WVAAERR

SEQ ID NO: 631 - protein, 12 aa, Artificial Sequence
  Laminin 421/521-binding peptide
MTHSAHNSQKTN

SEQ ID NO: 632 - protein, 7 aa, Artificial Sequence
  Laminin 421/521-binding peptide
WGLKLPH

SEQ ID NO: 633 - protein, 7 aa, Artificial Sequence
  Laminin 421/521-binding peptide
QGWFRHS

SEQ ID NO: 634 - protein, 7 aa, Artificial Sequence
  Laminin 421/521-binding peptide
IPVGERK

SEQ ID NO: 635 - protein, 9 aa, Artificial Sequence
  Laminin 421/521-binding peptide
CMNKIQRDC

SEQ ID NO: 636 - protein, 12 aa, Artificial Sequence
  Laminin 421/521-binding peptide
KALGYSLVGGEW

SEQ ID NO: 637 - protein, 12 aa, Artificial Sequence
  Laminin 421/521-binding peptide
APKVTNARPTQL

SEQ ID NO: 638 - protein, 12 aa, Artificial Sequence
  Laminin 421/521-binding peptide
AWVEAQNASNPS

SEQ ID NO: 639 - protein, 9 aa, Artificial Sequence
  Laminin 421/521-binding peptide
CSKHNHSRC

SEQ ID NO: 640 - protein, 12 aa, Artificial Sequence
  Laminin 421/521-binding peptide
GKPDHRLVSLWR

SEQ ID NO: 641 - protein, 9 aa, Artificial Sequence
  Laminin 421/521-binding peptide
CSHHKLKQC

SEQ ID NO: 642 - protein, 12 aa, Artificial Sequence
  Laminin 421/521-binding peptide
GGYDLPWANWQN

SEQ ID NO: 643 - protein, 12 aa, Artificial Sequence
  nerve-binding peptide
GWQMFPPMQNTR

SEQ ID NO: 644 - protein, 12 aa, Artificial Sequence
  nerve-binding peptide
SSLYMNGWSRDQ

SEQ ID NO: 645 - protein, 12 aa, Artificial Sequence
  nerve-binding peptide
YAGQNGWMRSDL

SEQ ID NO: 646 - protein, 12 aa, Artificial Sequence
  nerve-binding peptide
VERLNWPKRMQS

SEQ ID NO: 647 - protein, 12 aa, Artificial Sequence
  nerve-binding peptide
WDVAWLERRALH

SEQ ID NO: 648 - protein, 12 aa, Artificial Sequence
  nerve-binding peptide
VRLPMLASMGHQ

SEQ ID NO: 649 - protein, 12 aa, Artificial Sequence
  nerve-binding peptide
DRGLTILTDDLR

SEQ ID NO: 650 - protein, 12 aa, Artificial Sequence
  nerve-binding peptide
GWNEATNSARHT

SEQ ID NO: 651 - protein, 12 aa, Artificial Sequence
  nerve-binding peptide
EFARFRVFGVAE

SEQ ID NO: 652 - protein, 12 aa, Artificial Sequence
  nerve-binding peptide
NRIGHGTATGTL

SEQ ID NO: 653 - protein, 12 aa, Artificial Sequence
  nerve-binding peptide
VTDMLGIPKLHA

SEQ ID NO: 654 - protein, 12 aa, Artificial Sequence
  nerve-binding peptide
DELVGGAPWYKS

SEQ ID NO: 655 - protein, 12 aa, Artificial Sequence
  nerve-binding peptide
GLLREPDELMFL

SEQ ID NO: 656 - protein, 12 aa, Artificial Sequence
  nerve-binding peptide
QNIVENFIDNER

SEQ ID NO: 657 - protein, 12 aa, Artificial Sequence
  nerve-binding peptide
SGEYNLIKPNLL

SEQ ID NO: 658 - protein, 12 aa, Artificial Sequence
  nerve-binding peptide
EESNAKLELLSR

SEQ ID NO: 659 - protein, 12 aa, Artificial Sequence
  nerve-binding peptide
GWANSITYRNLY

SEQ ID NO: 660 - protein, 12 aa, Artificial Sequence
  nerve-binding peptide
NYAVGDRITKNI

SEQ ID NO: 661 - protein, 12 aa, Artificial Sequence
  nerve-binding peptide
HGIVFSKRPFSN

SEQ ID NO: 662 - protein, 12 aa, Artificial Sequence
  nerve-binding peptide
FDPLSRNVPHKT

SEQ ID NO: 663 - protein, 12 aa, Artificial Sequence
  nerve-binding peptide
NAVHRPDLNLLA

SEQ ID NO: 664 - protein, 12 aa, Artificial Sequence
  nerve-binding peptide
GMRTGDDSPKYP

SEQ ID NO: 665 - protein, 12 aa, Artificial Sequence
  nerve-binding peptide
IPGQTPRMPHSK

SEQ ID NO: 666 - protein, 12 aa, Artificial Sequence
  nerve-binding peptide
KTTSMTALTMGL

SEQ ID NO: 667 - protein, 12 aa, Artificial Sequence
  nerve-binding peptide
VNYQTPWNKHWY

SEQ ID NO: 668 - protein, 12 aa, Artificial Sequence
  nerve-binding peptide
VESGHAYSVSSW

SEQ ID NO: 669 - protein, 12 aa, Artificial Sequence
  nerve-binding peptide
VRSDMVSGGLQK

SEQ ID NO: 670 - protein, 12 aa, Artificial Sequence
  nerve-binding peptide
YANGIEAKDSSR

SEQ ID NO: 671 - protein, 12 aa, Artificial Sequence
  nerve-binding peptide
YAWDTVASRNYK

SEQ ID NO: 672 - protein, 12 aa, Artificial Sequence
  nerve-binding peptide
LQTHSITYRHER

SEQ ID NO: 673 - protein, 12 aa, Artificial Sequence
  nerve-binding peptide
SLGSSFVQGGEL

SEQ ID NO: 674 - protein, 12 aa, Artificial Sequence
  nerve-binding peptide
WEKHTGPLSRYL

SEQ ID NO: 675 - protein, 12 aa, Artificial Sequence
  nerve-binding peptide
AFRFEFQVPDSH

SEQ ID NO: 676 - protein, 12 aa, Artificial Sequence
  nerve-binding peptide
GHAHSLTQNPLF

SEQ ID NO: 677 - protein, 12 aa, Artificial Sequence
  nerve-binding peptide
GLTIMSSRHSLQ

SEQ ID NO: 678 - protein, 12 aa, Artificial Sequence
  nerve-binding peptide
NGMNWLQVHQLS

SEQ ID NO: 679 - protein, 12 aa, Artificial Sequence
  nerve-binding peptide
WSGGMEDNSNRL

SEQ ID NO: 680 - protein, 12 aa, Artificial Sequence
  nerve-binding peptide
AIMSYNSPWIQG

SEQ ID NO: 681 - protein, 12 aa, Artificial Sequence
  nerve-binding peptide
DVMQMFHQVHFY

SEQ ID NO: 682 - protein, 12 aa, Artificial Sequence
  nerve-binding peptide
QANLYYWDPSDL

SEQ ID NO: 683 - protein, 12 aa, Artificial Sequence
  nerve-binding peptide
QRATNDILIRGW

SEQ ID NO: 684 - protein, 12 aa, Artificial Sequence
  nerve-binding peptide
SPMANVWFNRLS

SEQ ID NO: 685 - protein, 12 aa, Artificial Sequence
  nerve-binding peptide
YSANSNPKKSLM

SEQ ID NO: 686 - protein, 12 aa, Artificial Sequence
  nerve-binding peptide
ALNHNGPVKTGL

SEQ ID NO: 687 - protein, 12 aa, Artificial Sequence
  nerve-binding peptide
DASQYPLVSGLW

SEQ ID NO: 688 - protein, 12 aa, Artificial Sequence
  nerve-binding peptide
EPWQSRDKVIEE

SEQ ID NO: 689 - protein, 12 aa, Artificial Sequence
  nerve-binding peptide
GLWDGKGRIVEV

SEQ ID NO: 690 - protein, 12 aa, Artificial Sequence
  nerve-binding peptide
GTVSYMVKMDNN

SEQ ID NO: 691 - protein, 12 aa, Artificial Sequence
  nerve-binding peptide
SLQLWNSEKVTT

SEQ ID NO: 692 - protein, 12 aa, Artificial Sequence
  nerve-binding peptide
WVGPDIIAQMRI

SEQ ID NO: 693 - protein, 12 aa, Artificial Sequence
  nerve-binding peptide
EIIVMSGSNVTN

SEQ ID NO: 694 - protein, 12 aa, Artificial Sequence
  nerve-binding peptide
HSGAYTIQASYK

SEQ ID NO: 695 - protein, 12 aa, Artificial Sequence
  nerve-binding peptide
MLKMERPGRMAY

SEQ ID NO: 696 - protein, 12 aa, Artificial Sequence
  nerve-binding peptide
SDHTFKRVMFRW

SEQ ID NO: 697 - protein, 12 aa, Artificial Sequence
  nerve-binding peptide
EMVPMKFLMMAK

SEQ ID NO: 698 - protein, 12 aa, Artificial Sequence
  nerve-binding peptide
NTEYSRYTSIWK

SEQ ID NO: 699 - protein, 12 aa, Artificial Sequence
  nerve-binding peptide
TDNGSFLGKLTS

SEQ ID NO: 700 - protein, 12 aa, Artificial Sequence
  nerve-binding peptide
TEWMVRASSIGE

SEQ ID NO: 701 - protein, 12 aa, Artificial Sequence
  nerve-binding peptide
VPVPKKMSLDPT

SEQ ID NO: 702 - protein, 12 aa, Artificial Sequence
  nerve-binding peptide
WPLKRFEQLPLK

SEQ ID NO: 703 - protein, 12 aa, Artificial Sequence
  nerve-binding peptide
MLRHVDLTEPNA

SEQ ID NO: 704 - protein, 12 aa, Artificial Sequence
  nerve-binding peptide
QSTLGVHMLRVS

SEQ ID NO: 705 - protein, 12 aa, Artificial Sequence
  nerve-binding peptide
RTVITNEMLLLV

SEQ ID NO: 706 - protein, 12 aa, Artificial Sequence
  nerve-binding peptide
SEPIEKGSRTIV

SEQ ID NO: 707 - protein, 12 aa, Artificial Sequence
  nerve-binding peptide
TLNARDQLSING

SEQ ID NO: 708 - protein, 12 aa, Artificial Sequence
  nerve-binding peptide
GTALNLQKDINK

SEQ ID NO: 709 - protein, 12 aa, Artificial Sequence
  nerve-binding peptide
SGWHLVMSMSIG

SEQ ID NO: 710 - protein, 12 aa, Artificial Sequence
  nerve-binding peptide
TSAHHDRTLAGS

SEQ ID NO: 711 - protein, 11 aa, Artificial Sequence
  JNK Inhibitor VI
RPKRPTTLNLF

SEQ ID NO: 712 - protein, 16 aa, Artificial Sequence
  SC 3036
KKHTDDGYMPMSPGVA

SEQ ID NO: 713 - protein, 28 aa, Artificial Sequence
  NEMO-Binding Domain Binding Peptide
DRQIKIWFQNRRMKWKKTALDWSWLQTE

SEQ ID NO: 714 - protein, 26 aa, Artificial Sequence
  NF-kB SN50
AAVALLPAVLLALLAPVQRKRQKLMP

SEQ ID NO: 715 - protein, 29 aa, Artificial Sequence
  TIRAP Inhibitor Peptide
RQIKIWFNRRMKWKKLQLRDAAPGGAIVS

SEQ ID NO: 716 - protein, 4 aa, Artificial Sequence
  Synthetic Sequence
LEHD

SEQ ID NO: 717 - protein, 4 aa, Artificial Sequence
  Synthetic Sequence
IETD

SEQ ID NO: 718 - protein, 4 aa, Artificial Sequence
  peptide linker
GGGC

SEQ ID NO: 719 - protein, 4 aa, Artificial Sequence
  Synthetic Sequence
KKKK

SEQ ID NO: 720 - protein, 4 aa, Artificial Sequence
  Synthetic Sequence
GGGG

SEQ ID NO: 721 - protein, 4 aa, Artificial Sequence
  Synthetic Sequence
LETD